SEQUENCE LISTING (2 sequences)

SEQ ID NO: 1 - DNA, 15910 bp, Homo sapiens
ctgtacagctctccgacaatcccacatctagatgccaagctgaggttggcattctcactaatttgctgttataaatattaagctatcataagcgttagcctacatatgactctttcatatgttagttaattattttagggtagaaatccaaaagtggagttaccagaagtggatatagacattctggctgggtgtgatggttcatgcctgtaatcccagcactttgggaggcagaggcaggcggatcacttgaggccaggagtttgagatcagcctgggccaacacagcgaaaccccatctctactaaaaattccaaaactagccaggcatagtggcacatgcctgtactcccagctacttgggaggctaagacacaagaatcgcttgaacccgggagggaggtggaggttgcggtgagctgagattgtgccaccgtactccagcctgggtgacacagctagactctgtttcaaaaaaaaaaagaaaaagaaaagaaaaaaatagactttctcttggctcagtgtatactgccaaattgttttccaaaaaaattgtgtcaatgtataacaccatcactaatatagtattgatattatggttattacattttaaaattcataatttgtaattataacattcataatttattactatttataatattaatgtaaatgtatattatatataaatgttatagtaattataactttggtagtgacaaagtattaatttattaggtgaagtatatgcttttttattagtgataataaatatatcctctctcccattataaaagtttgtatttcttcttttagaaattgattcttctgtcatttgcacatttatctgtataattataacagggtatttcccagtggtggctaatgagagaattatgggaaagtatagaacactattcaaatgcaaagcactgtatgatttttatttaataggaagacattttgtgcagcgatttctgattgaccacagtttgatcaagtgcatttgttaatgtgttctacattttcaaaaaggaaaggagaatttgttacattcagaacttgctgccactcctttgctacgtcataaagggtcagttgcccttgctcatactgacctattctttacctctctgcttcttctttgtgccagaagagtagaaatctgaccctttggggataccaccctctcccctactgctctctccaacctgaggcaaactttctcctacttcccagagcctgtcagaagtggtgaagccagcctgctccttggaatccagaactactttcagaatcttgaacttctgtgacctctcagggtccccttgtgtgaagtttttgacgtcagcttctcctgtgacccttagaagtcactcttgtgtctagcacatcccaggtgctcagtcaccattgaactacagtcatactatctcctggcaaaggctcttaactgtccatgttagcctgatattaatatcctggaagcttatactgtcgttcttccttccaggtttaaataaggcagcccctttatcctgtcacaggtcctctctccctacctatccttacctgttttggataacaacctttcttatttctaatagatttatttatttctcacatttccttcccttatcatagttttcctctcactttctcctctagtttgtcatactctggctttaaaacatgcaaacatgtgccttatggggaaaaaaagacaattttaatttaccttgcttcttctttacaaatgtattgtggcttcttcttatagtccaaatctaaaactctttacccacccactgccttgaactccttcctcgttgtgaaagtaggatggggcaaagagagaatgcatgcccctcccaactgctcaaacaagtaaaggtgctgttacagttatcttttgctaccttaatacaataattattttattatatctcacaattttatggatcaggaatttagactgggctcagctaggcgattcttctgctttactgacatcataggagatcacttggtggtattcaactgtcaggtaggcttatctggagggtccaagatagctgtactctggtgcctggtgccttggtaaagagggatgatgatgtggggcctctccagcatgaacagcctcagagaagtttgctttcttacatgctggcccagggctccaagagcaaatgttgcagtgagtaaagcagaagatacaaggacttttataatctggtctcagaagccacatggcatcagttctgtattattctattggtcaaaacattcataagcctgccagatgcaaggggaaggcatatgtaccctcatcttttgatgggaggaatgtgatggatttgcaattatgttttaaaactactacagacagaaccactgagaaagattcatgggtagctttggggtgaggactgggaattaacctgttgatagcagaggttcactagagtcaacaaggaataaggtctcctcttgtacactttagtcatactataccaacattcttaaccactgcttagccatcagcctcacaacataacaactccatcatagttgtactccctaagatcaccaacaatgttagagtcaaatccggtaggtttttctttgtttttgtcctcctgacattttttctaaacttgacactggtcagacccaatctttctttaatcatattcttaaataccagttctatcactggatatgttactgtttcttgttctcactctacctttgacaaagccattctttccagactataactctgggtctgggtccccctatggtttggcccttgaattcttttcctagtcctatttgactagccccattttcccgtgaaaagcatgcccctttcattgcatccatatcatgactaccaaatacctcctctatttcttcctcttttagcatgttaaatgcagcttcctaagctctctatctggatatcaacagtattctctccaaataattctaagactttaaaaattggtttaatcttcttacccctaaaatcaccccccttaccaactgcctcatgacaatcattggtactgtcactgagcttgcaacccatgttcttaaacatagagtaatctttgactccacatctaatcattcataaagctgtattgtctatcaaattaaatctgacatttatgtgagagcacttcatagtctgtaaagcactacacaggtgataacatgaagctacactcataatggatttgcaggctctgcttctcatttggcttctacagcctcatccctcaccaacttcttgccctacctctctctttcttccccatcacccaatttcccagtcagtcaggccaacagaatgcattctatatacgcgacttgctttccccaacatctttgcctgtatgcatgccacttatttgcctcagttgatctttatttcaacaagtgtttgcagaggagaaacctcgctggctccttctcctttctattttttttcagaggctacccgtcaggtcaacattgcctttttcagggaagctctgcaagcctgacctcccttggaagtgccttaggactggcttcttgcacagtacacaacctttacttatagagggtttggagattattctttattcatgtcttatttctcctgctcctggaggagatgactctgacttccactgactcttttggggggcttaagtcagggttgagtaccagaggccctaaatagctggacgtggattctggtaatatcaaatccatctttggcttaactgagaggttctgaaagctgggacctgaccttgtccatttccctctttctccagtttcctattatttcccactgttttttttaaaagttttttgttttcttaagttttcacaagaataaacattgaaaataaaatttgcacaaagatcgaactaggaaaggccacacaaccaacacatattacatcattataggtaagttagcagggagatttcagacctgggctagctctggaaccacattttacactgttgaaaataaaagctggagtacagatgactttcccaggttcacagagttggtaagctggagagctgcacctggagccaagcaacctgccctgtcctttccactgcaccctctaagaaatctaattagaaggaacaggtggtatctcattttgtacggtgctttagcaatgtactatttgctttctagtgtgtctattgtctcgtttgacatcttctctcaaaaagtgatgaaacgaaacgctctttttgacaagttcagagtgctcttggttcctgtgtgggattcttccaagtctgaatttggtagtgggaagagaaggaatccggaggaaggaggatgagaagtttaaaggagaggaaagggaagcagagaaggccgcaaggtgcctgcaagatgtctggggagttggaggaatggaagagtgccccgctcttccttctgggagagctccagctaggcagaacctttcaccaaggctctgatatcgtgctggtttccgaaagccccagccgaaggtgtgcagccaaagggtgacagaaggtgaggcacgtgcgggggcgcgggtgctgaccgccgcggtgcgccctccctccgacgtgcggtgtgcggggcgcagacaaccagcggccggcccagggctttcggggagcgaagcagggctcccgaggcaccgagcgagaatgggaatgggagggacccggtgctcccggacacgcccccggcaggtcccacgcccgggtcttctgagacctcgcgcggcccagcccgggagcggcccagctatataagtcccagcggaagaccggaacgcagagggtcctgctggcgcgagggtgggtaggaggggacgcggggactcggcccccaacaccgcgctccgtctgcagccgccgcctctgcaccgccgctgcccggcggtcggttcaaaaaacagaaatcgggtttgctgcccggcggacaggcgtgaagagcaagggaaaggaacttcctccaccttcggggctggagcccttttcctctgcatctccagtctctgagtgaagatggggggcctgacagcctcggacgtacacccgaccctgggggtccagctcttctcagctggaatagcggcgtgcttggcggacgtgatcaccttcccgctggacacggccaaagtccggctccaggtagctaggcagaggggtaagacaaggggtctcaggacagaggggacgctgttgcgtgcattccatttattctctgctttggtgtaaccactgtttctaggtagggtaggtgaccttccaaagcagtctggccttgtcccagggctggtgctttaggatgggaaactggaactttttctgggattagctgaagaaccaccagggccacagagaatgggttgaccatgactactaccaaattctcccaaaatttagggtgcacttagtattttaagagctgagaatattggcctctcctgagtttactagtcaggtgctttttcctttctttgattcttcgggggttctgtcctatcctactgccctaggggttctggagagttcctggggagggggatattcaaaatgtgcattgtagccagcctccctccatctgcgcgtgagcgaacacacacacacacacacacacacacacacacacacacacacacacacggtagagggaggtggatggaagaggaatgttgctgagaaaagaaacggaaaataggaacacagggggaaatcttggcttaagagtgaactcaatttcgctcccttctgttctgcacctttcttatttccaggtccaaggtgaatgcccgacgtccagtgttattaggtataaaggtgtcctgggaacaatcaccgctgtggtaaaaacagaagggcggatgaaactctacagcgggctgcctgcggggcttcagcggcaaatcagctccgcctctctcaggatcggcctctacgacacggtccaggagttcctcaccgcagggaaagaaagtaagccgtgagcgttcctgggaggggcagaaaagccttgggctccgctctgttccaaaaagtgtaacacacagaggagtggttttcataacaaattggcgagaaaacattcatatttgaactctcccttccccaaacattagctcattgttcatagaaaaaagtatgcaaaatcgattttttagatgcagatatatacttgtaaaggtcacccagtcatggaagttttgtgcccagtttggatctccatctggagaatatgggtgggctacagaaaaatgtttaacttaaagttctccaaagagggaagtatatcagaaacatctatggagcttgtcagaaatccaaacgaggactaccatggtcctctgagtctgaatcctcaggctagagaccagagtgtctttccacaagcttccctcatcatttgtgtatgcaacaaagttcaaagccttctgtttgaagcaaagaaagccagactttgtgaagagagttgaaaggacaggaaaagacatatttcctcttaagaggttcctcatcaggtccaggaaagaccagagcagaaaaagtggacgaatgctgcagggagtttgtttaggggaaaaagaaaaggaaacatatttcctgagtgccagtgcactctaagaattcctgtcactttaggtagcatttatttgagggcttaactatgaaccagacattgttctaagtgcttcagatacattataactggaagggtattagtaccattatcccttggcagatgggaaaactgaacacagagcagattcatcacttgcccaaggtcacacagctgggagggggcagagccagggttcaaacccaggcagtctggcctcggactccaggctcctaaccctgttctctactgccttctgcacttctcatatgattctgcccatcattcaaaccgcacaacactgctgtgagtaaaaagtgttagccgaatatcagggtagttaagtaacatgcacaaaatcacacagctaatcaacatcagaggcactttcatgtggagtagacaagccagagagaagatgtgctgatggcacaatgaatacattaagtgaaatccaccttgtagatttcatcatttctgctgtgagtaaccttcaatactataattttatgggataatttataaatgttgtctatacaaatatataagttatacttatccacacaagtactttcaaagtgaagataaagtctggatgttactagatcaaaactgcatttttttatttatagatgtagcaagagaggaaacacaaaggaggtaaagctgcccgttcaggtggttttcttcacagattgactgttctaccaattgttgtggactttgggcaccaaattaataggatatatgttggcagtgttctatgttatatagattcagtttatttagtaggctttattgaactgccatgtgccagtaactatgttagatgtttagatggcagatgtgtctctagacagagcttacagttgagagtatgggttgtgtggggagaagtgaatagatgactatattccatgatacatgctgtattacaatacagtcctacttcacttaacgatggggatacattctcagaaatgagttaggaggcaaattggttgttgaatgaacatcacagagagcacttacacaaacctagatggcatagccacacctaggctatatggtataatctattgctcctaggctacaaacctgtgcagcatgttggtattgaatactacaggcaattgttacataaagttaagtgtttgtgtacctaaaaatagaaaaggtaatgcattacactacagtcttatggggctgggatgtcactaggtgataggaatttttcagctctgttctaatcttacgggaccaccatcatgtatgcagcacatgactaactgtaattacaagatggtggctatattaaacagaactacttaagctagccatggaggtatggtccgtgagattttcctgaagaattaacgtctggatcaattctggaagggccagcaggagtactccaggcaaaggggtgagaaaggagcttccaagtagagtgaaggtcatgtgcaaagactcagtgaggagtcgagtgaacatagcacagggaggacatgttggtgaggaaggaggggtgaagccacagagacaggagggagccagatgacagaaggccttgcaggcggtgctaaggagtttggattttatccttacagtggtgggaagtcattgtaaaaatattaagcaagggagtggcataaacaatttacattttcaaaagatcactttggcagcagatagagtatatatgtaaaaggagtaagaaagaggtaagttagaaagcaagaaatgatcagggtatgccctaaaacactggcaatagggaaaaagagatgtcaatcagaaagattgagaaagtataattgaattgacttggtgaacaaatagaagtaaggcataagggacaggtagaaatatgagatgacttccaagtttctgtttaaagataccctttattgagagaggatgtatagaagctgtcttagggggaagacaagaaatttggtttaggccatgtcaacaggtaatggccagtaggcacatgattcagtttatttagtgggctccttttaggagaaaatctgagccagattccaggaagtcacagcagggactaccaatagggtcaaacagcagagagtgtggaaaggactgaaaagtgatcattgtacataacaaatagaagctcactgattttctagcaaaaacatcttcagcagagtagcgtggtataagctatattgtaggggactgaggaagaaatgggctctgagaagtaaagacaaacaatatgttttgtaaataaatttcttttagttcttaaaaaaaaagcctcttttccagcttgattgggaagtgaagagagggatttgaaagttggagattggaggataggatgagtacatcaagatacactacgttgtagtgcagtgcattacaaatgtgagctaaaagtgaaggcatttgtaatcatatgatattgctaattaaaagacagctgtcagtcatatgcccagctcctggtaaagcatgatgagaagagtacaatcatggtagtgatttaaaaattgctgccagttttgtggattttctttatgctagacagtgtaagctctttatcaatattatttaactcacacaactctaagaggtagatattattatccctttttgacaaattaggaaacagaattataatgactgagaaagtctctgctgagtaaatgttactgaaccttaattttatgtttacttaatgatagaaatgaatattgggcttcaagactatttgtacttaatgaaatctgtcttgagcaacataagctatttttttcaaaattttaagacaaaaatcactttcttctctcctgtcttcttatttttgttcccttcacatgttgtagcctaacactacttgatggcccattttggtgcagtttgtccactgggcttcatctaaggccaccaagtcccataattaacatgatcattcgtgggagaaagatcaagcctcattggtgatgggtgcctcctcacagtcggataatactgaaaagagagctaaatgtgggaaagaaccaagttgaacacaggaaagaatcaggccactgtgaaaataagcattgtgttttcttgttccttgaaagtcttcatttttaaaaaatttcagacacctgaagttttctagccttactctgagttgacgcacatttagtacatgatcaacacataaacaagcattagagaaatagaaaagctgtaagaatacaaaaatatgggccaggtgggtggctcatacctgtaatcctagcactttgggaggccgaggcagacggatcacctgaggtcaggagttcaagactagcctggccaatatagtgaaaccctgtctctactaaaaatacaaaacttagcaggctgtggtggcacgtgcctataatcccagctacttgggaggctgaggcaggagaatctcttgaacccgggaggcggagattgcagtgagccaagatcacaccactgcactctagcctagataacagagcaagactccatctcaaaaaaaaaaaaaatacaaaaatatgaaccactgaaaattaaaaagacatgcatgcattctaggtctttaattttttttcttaataattttttttctctctggatagcagcacctagtttaggaagcaagattttagctggtctaacgactggaggagtggcagtattcattgggcaacccacagaggtcgtgaaagtcagacttcaagcacagagccatctccacggaatcaaacctcgctacacggggacttataatgcgtacagaataatagcaacaaccgaaggcttgacgggtctttggaaaggtaactaacttcaaaatgggttttataaccaccaaagcacatacatacaactagcaacttattgtaaagtagagttaataaacattttctttttttttttccccagggactactcccaatctgatgagaagtgtcatcatcaattgtacagagctagtaacatatgatctaatgaaggaggcctttgtgaaaaacaacatattagcaggtaacttcccatttcatataacaaacaggtctgcacctttagaagttcatcttggagcttctgcagccaccttatactcaatctcttaactccaatagttttctctttttaaaaattaagtaattttgaaccatatataactttgtgagaagcaggaaaagaccaaaatattaagtttaagaagttttgccacaacaaaaatattttgcaacaaaaataacaggcaatttcatgtcagcattattctcatttaatactaatatatgggacttttgttagaatcttattctttatacagcagaattcaggaggtaagtccatcctgcatactatatccaaaagatctagttataaaaggagcttatcagtggtctcatccaaaaagtaataccataagataggttcttaaaaataatattctaacaacttctagagacattgaaatttcccttatttcaataaaaaagtattagatgctcatatattaggcattattacaggccttaaaggcacagaggaaactaacagtttactttcctaaagtgttaacaatctattaagccatttactctttaccttctttttctagtgcaatacctttcttattttattttatttatttataagacatcttcattgacctactgttatcaataggtttataaagatatgacagataactaaattgcaagcccccaaaagtctgatgttgacctgtttcatcgatccattttagatgacgtcccctgccacttggtgtcggctcttatcgctggattttgcgcaacagctatgtcctccccggtggatgtagtaaaaaccagatttattaattctccaccaggacagtacaaaagtgtgcccaactgtgcaatgaaagtgttcactaacgaaggaccaacggctttcttcaaggggtaagatatgatcttgtgtatctgtaatgtgttctggctgtctgtgtgctttgggacactctcatgtcaagcaaccgacatttagcttacaagccttagtatattcatatacttagtattgacttttccttgccacagatttctccaatccaccaattccactgtgccagaaagtaaaaagccatgatattcaaattttctcaactttgatcaaaggctcattcaagaccagtgccttttccactggtcccaatctactggaaatgcagacagtattttgccttctctgggcaagaaagttataaagtagagggaaatcataatagagagctatgagagaacaagatttgatttgatttaatttgatggactcaagttttaacattgtaaaactagagataagacatcaccaccaatctagaaaagtgatgcagaaaagtatttgatttgggtaattattacactcacctagaaacaagtgttgtgtaatagattacatatttccataatgcaatgttgtatcagaaactaccttcctaagaaaatatagtatgggctcggcgtggtggctcgcacctgtaatcccagcactttgggagatggaggcaggaggatcacttgagcccagactgggcaacaaagcgagaccctgtctcaacaaaaaatttaaaaattagctgagtgtggtggcacgcactgatggtcccctctacttgggaagctgaggcaagaggatctcctgagcccaggagttcaaggtttcagcgagctatgattgtgccactgcactccagcctgggagacagagcaagtccctgtctcaaaaaagaagaaggagaaggaggagaaaatacagtattaagtaatctgtcaatatattccacaaggattacactagtggtttaataataaaattatattacctttttaaattgtaaggccattcctcaagctttataaattaagcatgaatgcatcatacacattttataaaaagttccaactcatcataatctgtacttatgatacattaatacaaatgaagttcattataaaattaacttaaaatggatataccagttattaaaccattaaccatttaataattttatttttttcaaatttaaaaaccttttggggaagaaatactacaacatggatgaaccttgaaaacgttatgctaagtgaaataagccagacacaaaaggacaaatactgtatgattacacttaaatgaggtacctagagtagtcaaattcatagagacagaaagaatagaagttaccaggggctggaggtaggaaaaaatggagagctgtttaatgggtagagagtttctttttggggtgacaaaaaggttctagagatggatagtggtgatggttacacacaatgtgtgtgtacttaatgctactgaaatgtaattttatgattttttttttttgcagcaaaataccccacattgggaagtgaagagaaacatgttaagagacttgaaggaaaaaaattggggcagaggggtgttttttataggttaaacaataaaagccatttaaacagtaacaatttctctaaggacaagaatcgtcaagattgagacagcactgatttcttgactctactcaatacttctttggtttctcttcttccttcccccttctaatagtttcctacctcccattcagaaagcaaagcaaaacaagcaaaaattcccccttccctcaaaaaaggaaagagtttttgaaaaagttcatgtcagtgaagaaaagacatgttttgggagtgaaggatatttgtggatttgtatagatgtgatcatcagggctgtgttgttttgaagtaatataggacatctagaggaaaatttattttcagcagaggagggaaagatgaagagtaggtacttttaagcatcttcacttgaggagtggcaaaatgagaagcataacctgctataatcactttaagaatttcaggctgagtgtggtggtgcagtctctagtcccagttactccaggaggctcaggtgggaggatcacttaagcccaggagctcgaggttgcagtgagctatgattacactactgcattccagcctgggcggcagggtgaagcctcatctcaaaaattaaaaaaaaaaaaaatcaaacaaattaatcgaacgatgacatgcacttttctaggttggtaccttccttcttgcgacttggatcctggaacgtcattatgtttgtgtgctttgaacaactgaaacgagaactgtcaaagtcaaggcagactatggactgtgccacataatcagcttcaagaaaatgatgtaacataccagtgggaatcttgctgactggatcataaaaacaaacaaaacttattcacttattttaacctaaaaagataaaggaattttggcagagaattttggacttttttatataaaaaagaggaaaattaatgcctatttcatataactttttttttttctcagtgtcttaagaaggggaaagcaaaacattcagcatataccctggcaaatgtaatgcagataagctactgcatttgaccatttctggagtgcaattgtgtgaatgaatgtgaagaactttaacatgttttaattacaattccaactggtggaaaagaaactgagtgaaatgcagtttatatttataaatacttaaaaatgaagttattaaaaatattagtttttattaaccacagttgtcagttaatatattcaataaagtattgctaataccttttaaagtttgtcttttgagatctatacctgggtgtaagagtcaagttcactagaatacaagactgcccaatagcaaatgcaggtctttagaatcataggcatgaacctactctgaatgttattagtatagatttttaatgtttagagtccagatttgatgacatctctaacaacttctaatctaagacactatattcattttggcaggattgctactagagtcttggtatctgtgctagcatcacataattttagagctggagggtacttctgggaagacagaggaacagtttgagattcctactgagatgaaaacgaatcttcatggaatctttcagcaaagccaaattcaaattcatcattagcacctgtagtaaccttttcaatgcctacaaactgcatgcagaagagatagggaaacagtaaaacagatattaaaagaagtttttaagacaaagcccagcctgattttaagctaaatccaaggattggcagcttggatgagcaggaaggttacaggctgccagacatcattctagttctgttttaatcaactccatgttacatttactatcagggattctcacctcaccctcatgcatgtcttccccattcattacccgcaaaagtgtcttgtagcagatgtcttctgtgtcccatacataccattttgctctttagtgcttgctggcctgacttcctattgtcatgtcagcatctgccctttttagggtctctggccaccagagccagctttactcacctgtgcatggcattctagaagagcagcagggaaaataacacagccccagtgcagcccttaaccaccaataactggtagtagttggtgtacaaatatctcagttccctcaactgtcaggtggaataccgctgagggatcaaactctagtaacacacagtagtgttttgcttactatggttaactaaaaaatcacagggtcttcatgcatttggaaaggatactttatttcttacaaagggttacagcctacaaggtggtcattctgcaggctagaaagcgtaacctccagcaaagaccggaggcaggcacttctagggaaggaagagtaagacagaaatttaaattgaatgggttggccaagtatacatattcaacaggctacaggtggattcatgaatattcatgaaggcagtcctgatgcatgcatgttacaccttggggtggaggcttaacatttaaatgtattacagttaggccctatacatgaaaaggtgaagcagtaacacgaaggcacacaatgcaccatttctgtaaacaggccagagccagttcacagtggttggtctcttatcatgagaaagctactaaaatcctcttgtccagttaaaactgtagttatggctggtggaaaatgggctggagtcagtcaacacttggtgaagctgcagttgcttcagacactcaaggccagtgtttgtttagctgctcgagaaaaagaaaaatcttgtggcagttagaacatagtttattctttaagtgtaggagtgtgtgacttaa

SEQ ID NO: 2 - DNA, 15174 bp, Homo sapiens
tccagcctgggcaacaagagtgaaactcggtctcaaaaaaaaaaaaaagagaagaagaagaaagaaaactaggtggagtgtggtggcttgcacctataatcccagcactttgggaggccgaggtgggtggatctattgaggctaggagttcaagatcaacctgccaacatgacgaaaccccacctctactaaaaatacaaaaaattagcacggcgtggtgtgtgtgcctgtaatcctagctacttggaaggctgaggcaggaatcgcttgaacctggggggcagaggttgcagtgagccaagatcttgccactgcactccaggctgggcgacacagcacaactctatctcaaaaaaaaaaagaaaaaacaaaagaaaactaatatatcaaaataatttctagttagttggattcactcacttattcattcaatgacttattgaattatcatatattactagtgctttttaatacataccttctacaatttttcaactgaaaattacttcattgatcagggctctttaaactgatctccatttgcattgttttactaactatagttattattcatgtattagcactctgagcctactgtaatgatgtgtaccttaataaagaactgaatatttgtaatggctggcagtgaatttagtagttcttgaatttagagctcaaaatatgggagtaatttgctgctttatttcctttgagaggtaatagaggaaaaacagaatctaataacaatcacagattttcgggaaagcactgtaaaaccatatgatcaattctagcttcttatgtaaacatggaaagattgccagctgaacacctgtcatgctctaagaagttggggagaatttgcatttttagaactgtgagcaaaatgagaacgactgctatgttcatgctttgtgaatttagctttatttcattcacacaattcatgggaaaaaatgcatcttttaactcggtgtttttcaattcaacttttaaaatacaggagtgggccagacccggtggctcacacctgtaatctcatcactttgggaggccgaggcaggtggaccacaaggtcaagagatagacaccatcctggccaacatggtgaaaccccatctccactaaaaatacaaaaattagctgggcatgttggcacgtacctgtaatcccagctactcgggagactgaggcaggagaatcgcttgaacctgggagatggaggttacagtaagccgagatcgcgccactgcactccagcctggcgacagagcaagactccatctcaaaaaaaaaaaaaaaaaaaaaaaaaaaaaaaaaaaaaaaccaggatgtgttaccaaggaaaattcatttacaatggttaattatgtgacaaacatgtcaagtaattccatctggctttgtgtcaccatttccccacccttttttcagaaaccaaaaccaagaagaagaacaaacatcaaaatggacatggaaattaacaaatatatgattcaatttaatctcctaagaggttttttaaaattattttattttgagacggagtcttgctctgtcgccaggctggagtgcagtggcaggatctcagctcactgcaacctccatctcccaggttcaagcgattctcctgcttcagcctcccaagtagctggaactacaggcaagcaccaccacacccagctaatgtttgtatttttggtagagatggggtttcaccatgttggccaggatggtctcgatctcttgacctcatgatccacccgccttggcctcccaaagtgctgggattacaggtattttttattttttttgagacagggtcaccctgtcacccaggctggagtgtagtggcacaatcatggctcactgcagcctcaacctcccaggctcaggtgatcctccatgtcagcctcccaagtagctggaactataggcgtgcaacaccatgcccagctaatttttgtattttttgtagagacagggatttgccatgttggccaggctggtcttcaactcctggcctcaagtgatccacccgtctcaacctcccaaactgctaggattacaggtgtgagccaccgtgccccatctcatctgctaagtgggtttaaagaaattcagtttcatgtcaatttttaaaatgtatggttatcaaattcgacttctttttaaaaatgcaatcagataactgtatgcttgtttgatgaggggaggaaagttaatatagccaatctactcaatatttttagcagaaattatcagagactaaggaaatgtttaagtttttctcatgttggttttaattacctaatgttttcagttttctctttcattcttgtgtctttttttcattttcagtgtttcaaatacagtttgtatttaaagatttagaagttccaaaactgtaagcacagtggattgtttcctgggatgatgttaaaattatacaacaaaatatatgaaactttgtcaatttggttattggcacatacaaaatatttacaaataaacgtgtgtgtgtgtgcgtgtacacacaattcaatgaaatagatgtgaaacaagttttcttttttttttttttgagacagagtcttgctctgtcgcccaggctggagtgcaatgtcgcagtctcagctcactgcaacctctgcctcccgggttcaagcgattctcctgcctcagcctcccgagtagctgggactacaggcacctaccaccactcccagctaatttttgtgtttttagtagagacagggtttcaccatgttagccaggctagtctccaactcctgacctcaggtgatctgcccgcctcagcctcccaaagtgctgggattgcaggcgtgagccacctcacctggctacaagttttcaaaatacatttatctgtacccatacattctccagtttgtccacaggacatcttatgacttgagcaagctgctaaaaatccaagggtgcagcgtttgtatgtctataggattgctcagatctgcccccaccctgaaagaatttaagagaatttcttgaggccaggcacagtggctcacacctgtaattccagtactgtgagagtccgaggtcagaggactgcttgaggccaggagttcaagagcagcctggacaacatagggagacctgtcactacaaagaataaataaattagccaggcttagtggctcatccctgtggtcccagctactagggaggcagaagtaggactgcttgtcccaggaggtcaagactgcagtgagctgagacccagccacctgcattccagcctgggcaacaaaaagagaccctgtctcaaaaaataagttaaataaataaataataaaaatagtttaaaccctaaacacatcttctttttcaaagaggacttcttaaggacttcatgctgcgtcctgttgatctccacttccctttttcagcgtccacacttttaacagtctcttttgccaaggataataagtatatagtttctggaatccagattcttccctgtttggacagccagggggacaatttttggtctgcaggcctttgcatctgttctgctgttgctcagcaatctcacagcaaatttgccgagcctctccggaatgcacagccagacagagctcagcgcaaaagctagagaacctggcggagggagactcacagtgccacaaaaaaactttatcttttctttttttttttcttttctttctttctctttctttcttgtctttctgtctttcctctctctctctctgtctttctttcctctctttctttcttttttcctacatggcaagatctcctcatggcagaaataatctgccttgacttctgtttccacgctgcttctgccaggaccatgcgctcggcgtgtttttctttccgctataattatccaggcccatcccagctctggtcccctcagctgttccctggcagtcccttctgctggtgaaaacacatatggcgccggcctgaccagggtgtaagtgtgtgaatatcaggaagatgactgaacgtctttgggactccgtttcctcattgtaaaatggaggttaataccagccttcttctactccccaaacgcacgtgtttgtcccggccagagggcccaattgttggctgttcacgcgtcagttacccccacaggacgggtcagccaattaaaggcgaaccaggcccggtccatctcctgacgccttttctcatcccagggctggacaggcagctggcctgggcccggctctgccttgtcacgtgcgggggccggcccgtttgcttgtctgtgtgtaggagcgtgaggtcacgctgggtgctcccgccccgccggggcctttagtgtcctggtccctaaacgccaggccgctccaccgggggagaaggcgcgaaccccagccgagcccaacggctgttgtcggttgccgggccacctgttgctgcagttctgattggttccttcccccgacaacgcggcggctgtaaccaatcgacagcgaggccggtcgcgaggccccagtcccgccctgcaggagccagccgcgcgctcgctcgcaggagggtgggtagtttgcccagcgtaggggggctgggcccataaaagaggaagtgcacttaagacacggccccgctggacgctgttagaaaccgtcctggctgggaaggcaagaggtgtgtgactggacaagacttgtttctggcggtcagtcttgccatcctcacagaggttggcggcccgagagagtgtgaggcagaggcggggagtggcaagggagtgaccatctcggggaacgaaggagtaaacgcggtgatgggacgcacggaaacgggagtggagaaagtcatggagagaaccctaggcggggcggtccccgcggaaaggcggctgctccagggtctccgcacccaagtaggagctggcaggcccggccccgccccgcaggccccaccccgggccccgcccccgaggcttaagccgcgccgccgcctgcgcggagccccactgcgaagcccagctgcgcgcgccttgggattgactgtccacgctcgcccggctcgtccgacgcgccctccgccagccgacagacacagccgcacgcactgccgtgttctccctgcggctcggtgagcctggccccagccctgcgccctttgcgccccccacgcttgttctgcgtgcgctgcccgctcttccatttaccttctctcccacccaagtttgtactcttttctttctctcggttttattttttgtttttgtttgtttgtttgagacaggctttcgctctgtctcccaggctggagtgcagtggcgcgatctcggctcactgcagcctccacctcccaggttcaagcgatccgcctgccgagtagctgggattacaggcgcccgccaccacgcctggctaatttttgtgttttgtagagatggggtttcgccatgttggccaggctggcctcgaactgctgagctcaagcaatccgcccgcctcggcctcacaaagtcctagaattttaggcatgagcctccgggtccggcctgtgctaatcctttctgtccttggttctttatttctcttctctctttttcttagtcccttttgttctttccctctcccgttcagttggctgtcgtttgagcctccaccttttcactccctcctttccaccacgatgccgagccctgccttggatggggaccatcagcgatgaccacaatgacctctcccttaccaggcagctccaggcagtgttcctgcaccgcctttcccagggcttgggggctttttctagtgggctttgagctgctcaatctggcctctgcagggccggctcccagcccttccaacctcctcacagcccgacctgggacctagccaattcccggagagtctctgtcccatcgtgaccccctcacaactctcccactcaccaaagtctgatgactgtgctagggggtgcttatatagagtactgagtgttacaaaagcagaagtctggatgagaaccaatttgtgatattaagcaggtggggtgggggtggggagtgtacctaggttcattttccgccctgcttttcccctttccagtgtgtgcacttaaccagtccctgggccctgttccccatccccctccaaggcatggattgggtgggcttgtgtgtcttggggcaggtggccctttctaaactctctgcctttgctcacccacaggacacatagtatgaccattaggtgtttcgtctcccacccattttctatggaaaaccaaggggatcgggccatgatagccactggcagctttgaagaacgggacacctttagagaagcttgatcttggaggcctcaccgtgagaccttacaaagccgggtaagagtccagtccaaggaagaggtctcttgctgcctcctaaccctgtggtctaggggcaggagtcagcagggcattaacaaaaataattaccatccccacccccgacagtgaagtggctctttccagttcacagagcactctcacacctccccgctctcattctggcccttcagctgactcggacaagccaaggatcttggtccccattttataaaggagaaaactgaggcccacgtgtaacagtgattggccccaagtcatcccgggagccagcagaagagctaggacaggaacctattgttctaacttcatattgatgctagcttttgactatccctgaaaccgagattggtaatcagcccggctctgaaactggttatttgctggggactgtaaaataggattaactatttctagtcctgcattttaattgctgttagtagggccatcttacccaccctctgaaggacctgacttggcaagcccaaggcaacattcagaatatggcagctgaacctctgtgcacttgtctttgggcagcagctgggtcttattcttctctggccttcacaacatcctgcaacccagctcaaggtcaggaatgtgacagactcatgtcatcatatctctgatgcccagagaagggataccatttgcctgagccttctcagtactgtttaatcagcctgtgagaactttccttgtgaaaggccctgtctgtgcctggggctgataaaacagcaagaacgaactgaggagctgggcagcagtgcaaagcaaatactaccagctttggtgcctgtaagtgtggctcttactcatctcacatggaaataagggcagccaccttgcagggctgctctgaggattgagctaatacagtgccctgggcgttggggtggggaaagttgtggagcacctcctgggggaagggggtgtcagagcagggaatctggggagtccgagggcaccttcatcaacccaatctgtcatttgagcaccagtcttcactgagcctcgtgggcaagctggagggaaacaggaataaggtcaggccctgttctataggtcccagtgtagttgctatggtgagtatcttcatttccctgcttgccccagccacctggagtgagaagcccaagaggaagctgggtgagctgtttgtttccatgggtctctgtgttcacagctgactcccttcaccagccagccctttcacctgagccccagcaacaaaggcagtcaggcggggctcaaagcagctgctccaatgaagtcaaagaaataagctcaggggaagaagcaggtcaccctcccccactagggtgctgggctcacttcctcctggggcagtggaggagggtgtggttccaactcagaacaaaatggggcttttggtttactttatcactcttcacagctctgacctggacccctcatccctgcctgtcttgtggtgtaagtgcggatccccctaagttggaggaaaggaaactggcccaaacaaaaaggagagcagttttctctgcatcacatggtaggccaggaggagtctaatgccccagagtttactctcagcccccaaaatcacctagctaaatgttaccttatctaagaagtccttaggttttttggggtttgttttttttttttttgagacaaggtctcactctctcacccagactggagcacagtggcacaatcacagctcactgcagcctcaacctcctgggctcaagcaatcgtcccaagtagctgggactataggcctgcaccaccatgtccagctaatttatttttatttatattttttagacagggtctcattatgttgccctggctggtcttgaactcctgggttcaagcagtcctcccacctctgcctcccaaagtgctaggtttttttttgtttgtttgtctgttttttgaaacagagtcttgctctgtcgcctaggctggagtgcagtggcacgatctcagctactgcaacctccacctcctgggttcaagtgattctcctgcctcagcctcctaagtagttgggaatacaggcgtgtgccaacacacccagctcatttttgtatttttagcggagatggggttttgccatgttggccaagctggtctcaaactcctgacctcaggtgattcgcccgcctcagcctcccaaagtgctgggtttacaggcgtgagccaccacacccagcccaagaagtcttttctgatcacccactcttccttctctcccaatggcattagttgttccctcctttgcattttgagagtatgtcctgtaagccccaaatgcagcttgaatcatctgcccatccaccccctgtgcccaacagtaagcctcctctagagtagatactatctcctgcatctcagtgaaccactgcccagcaaagcagtcttgctaaaacaatgactctagagatcctaagctgtgtgagagctggaggagagaattagactgatggtctgggaagggattgaattagtcatcttgtaccttttcttcttgacttaagttccagacctgtagcaaccattcctgcttagacatccagaacataagcctatgggtctgtgcctgttgggtcttagtctgggtgaaacttttctctacttctgtcagctctccagatgaaccacagaagcaggaatgtgggcatcatcagtgaaatctctgcatacagcagacaaagggctggtccagtggctgtttatgaggcagcgctaggagagctctgatccagactctccctgcagtgaaagggagggagcccttcatgaagtattgactgcttgagcaggaattgcttcaccagcacctaactgagtgcctctcgagctcacatcggttttccctcatgaggccacttggagtcttgctgagggacttggttctattagggaaggtgagtttggggatggtgagcagggagggcctggggacattgtggctaatggggcttttctcctcttggcttagattccggcagagttcctctatctcgtcttgttgctgattaaaggtgcccctgtctccagtttttctccatctcctgggacgtagcaggaaatcagcatcatggttgggttcaaggccacagatgtgccccctactgccactgtgaagtttcttggggctggcacagctgcctgcatcgcagatctcatcacctttcctctggatactgctaaagtccggttacaggtgaggggatgaagcctgggagtcttgatggtgtctactctgttccctccccaaagacacagacccctcaagggccagtgtttggagcatcgagatgactggaggtgggaagggcaacatgcttatccctgtagctaccctgtcttggccttgcagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcgtgagtatggagcaagggtgtaggccccttggcccttttttctcagtgatgattgatcttagttcattcagccatatagttttttaggccccacgatccctaggaagatcaggggaacagagaactggaaggggccctggtcctccacatagttcctaagcacctgggctataccaggctctgagcagggcgtcatcccatcacagtcttcaacaccaccttgggagtaggtagtatcatcccagtgttatagaagaagagactgaggtgggaaggcagtgggtagagtggggacttggccaggggcacacagtagagagccagaaaacacacagtagagagccaggacactcgtctctaaggccagcgttcttccctttcacctccttagtatgccatgccaaccctccattttacacatgacgaaacagagccccagacaaaaggttgtctttcccagatcacatggcaggaagaagtaaagctgacctgagatcccaagtcttaggaatcccagtcctcagaaagccacttctctctgagccttggttttcacatttgtcagatggaaatgattgtgatttctcagggctgttgagcaggtaaatgaaaatgttttatgaaagaaagcaccaagtttcattttggtcttagcccttgctatgtccctagcaagaagtagatattcatagggatattttgtttgatgtgaggagttcttacagcaagagcttgtagaaggccaaaagcttctggattctattcccaaaagcaggagatgacagtgacagggtggttttggtgaggagagatgaggtagaaaatgagtgcaagcccgctggccactgaccccatggctcgcccacagatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaaggtgtgtaccagttgttttcccttccccttttcctcctccccgatactctggtctcacccaggatcttcctcctcctacagggacctctcccaatgttgctcgtaatgccattgtcaactgtgctgagctggtgacctatgacctcatcaaggatgccctcctgaaagccaacctcatgacaggtgagtcatgaggtagacggtgctgggtctcacccttcccccatgccaggagcaggtgcgggggtctagctgacaccagaagaccacatcttttcatcctatttgccctttgcagggagagtaagatatctcttacttgccatattgaagccaattgggatgaagctcccactttgcacattgaggaactgaggctagattggcaaaatgactctttcaggtcctcagaagatgtctcagctggagtccctgtctgtttttgtttttttgtttgtttgttttttgttttttttgagatagagtctcactctgttacccgtgtaatctcagctcactgcaaccttctcctcctgggttcaagcgattcttgtgcctcagcctcccgagtagctgggatgacaggtgtgcaccagcacactggctaatttttgtatttttagtagagatggagtttcaccatgttagccaggctggtctcgaactcctggcctcaagtgatctgcccaccttggcctcccaatgtgctgggattacaggtgtgagcctctgcgccccatcctcttgtttgttttttgagacagggtcttgctcggttgcccaggctggagtgcagtggggtgattaatggctcattgcagcctcgacctccctgactcaagcaatcctcccacctcagcctcctgagtagctggggctgactacaggcatgcacactgtgcctggctaatttttgtattttgtagagacagggtttttgccatgttacccagtctggtcttgaactcctgggctcaagtgatccacccacctcggcctccaaaagaagtcctggattacaggcatgagacattgtgcccagcctctctgtctctttaaaatcatgaaaactcgtagctacttaagtaattctcctgccttctggaatgatgggtgaagatcttgactgccttgcctgctcctccttggcagatgacctcccttgccacttcacttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggtgagcctctggtcctccccacccagttcaggcctcttggctatgcatgtctattgtgggtgggagagaaccacctggaagtgagtagcagccaagtgtgactatttctgatcctggtcctggcatttcaccagcattcacctatccccttaattccttcctcccagaattgctaccatcactgtttattaggtgttaaatggagactcaaagggaattcatgcttatagccaagcagctgtgagctcagttcattgagtcctcccagcctcctttgggacagagcaactgggttggattgaataccaggcccagtgagggaagtgggaggtggaggtgcccccatgacctgtgatttttctcctctaggttcatgccctcctttctccgcttgggttcctggaacgtggtgatgttcgtcacctatgagcagctgaaacgagccctcatggctgcctgcacttcccgagaggctcccttctgagcctctcctgctgctgacctgatcacctctggctttgtctctagccgggccatgctttccttttcttccttctttctcttccctccttcccttctctccttccctctttccccacctcttccttccgctcctttacctaccaccttccctctttctacattctcatctactcattgtctcagtgctggtggagttgacatttgacagtgtgggaggcctcgtaccagccaggatcccaagcgtcccgtcccttggaaagttcagccagaatcttcgtcctgcccccgacagcccagcctagcccacttgtcatccataaagcaagctcaaccttggcgtctcctccctctcttgtagctcttaccagaggtcttggtccaatggcctttttggtacctggtgggcaggggaggaaccacctgactttgaaaatgggtgtgatccaccttccacctccagcatccaatctgaagcccgtgtaggtcatctggtccatttctctctagacccaggccctgtactaacatggggagtgcaggagccacctgagagacagcagtgcctccccttcctttgccgggccacttgagctcttactcagaatctggtactctagtgcctgccatcccaaccccccaccccagccgcaggcctgtttatctgcacaacaagagtgctcctgtgtgccctgcatctcctgcagttccagaggaacatgagactcttagatgctgttgactttattttattccattttacaaatggaaggaagacccacctcccccaaagtcccagaccttgtgagaacaagtcagtcagcctccttccaccctccacagccacagccacacccacagaggaaatgttactgaactgggtggagcaggccctgactccacagagggtgggtggaggctgcagggcaaacatctggtctctgcctgaggatactttccatttgtgttttttgttgttttgagacagagtctcacttgctgtcacccaggctggagtgcagtggtgcaatcttggctcactgcaacctctcccaggttcaggcgattctcctgcctcagcctcccaagtagctgggattacaggcatacaccatcatacctggctaatttttgtgtttttggtagaaacggggttttgccatgttggccaggctggtctcaaactcctgacctcaagtgatccacctacctcagcctcccaaagtgctgggattacaggcatgagccactgtgcctggccaggatattttccatttggagtctcaccaccacaacccccctccacctgcccctgccccagctaggcatccaaggaggccgcaagaagccagggccttggctgcacaggggtctccgcttctctgtccctgttcttatcacctgcactcagaggcaggtgggcaggggtactacaatttcaaggagtggagactgtgaggtcctggaatcccaaggcatctcctgtagggctgggcccttagaattatgtcactcagacccagtttgtaggtgtctgaagaaactgaggcctgacacaggtgatgcaggcaagaacacccagaaagtccactactgaactgggaccgggacccagtcctccttccccttgtggactcccccagagaccagtgctggggtccttggggaagcctgtttggcagctgtggagctaggccctgagaacacgaccaccctccctcttccctcagcctcaagccgctgaagccactgctgcttcgccgcctcgtaagcccaatggtcagagctggaggctagacccttcagtgcttgggttgagggccagggtgttagattggtttttggagaaggaacgagggcccaggattcttcagcttcttagtttttgacaaattgagctgaggccccatagtcctcgggagggacagggttgagtgccataagtcggcaaaccagggtaaaggtgacaggcagctcagccaggctgcagggggtggcatatacagaggacctggccactactttatgtaccttcttacactaattctgtgaggcaggctgtttgttagctctgctctggacgggaagaagtaggggcagtttggtaggtgtgtgtcaaagctaaacaggctgggtgggcatgagcaagtcagctggttcattcagcagccttaatagacacgaggctacccaacttcactgtggttctgggtgtggccttaggacaatgagctgggaacagtggtaggaaccactggaaaacataccagtgggtctcattcattctgatcacaggtagatcacttctctttggttcccaaccctttaatgcctattaag

(19) United States
(12) Patent Application Publication
(10) Pub. No.: US 2015/0216892 A1
(43) Pub. Date: Aug. 6, 2015

(54) CELL-SPECIFIC DELIVERY OF MIRNA MODULATORS FOR THE TREATMENT OF OBESITY AND RELATED DISORDERS

(71) Applicant: APTAMIR THERAPEUTICS, INC., Austin, TX (US)

(72) Inventor: Marc Thibonnier, Austin, TX (US)

(21) Appl. No.: 14/419,253

(22) PCT Filed: Aug. 5, 2013
(86) PCT No.: PCT/US2013/053613

§ 371 Date: Feb. 3, 2015

Related U.S. Application Data
(60) Provisional application No. 61/679,461, filed on Aug. 3, 2012.
(60) Provisional application No. 61/681,739, filed on Aug. 10, 2012.
(60) Provisional application No. 61/695,477, filed on Aug. 31, 2012.

(51) Int. Cl. A61K 31/713; C12N 15/113; A61K 47/48; A61K 9/48

(52) U.S. Cl. CPC A61K 31/713; A61K 9/48; C12N 15/113; A61K 47/48092; C12N 2310/141; C12N 2320/32; C12N 2310/3519

(57) ABSTRACT

Compositions comprising microRNAs (miRNAs) and targeting agents are disclosed, as well as methods for delivering a therapeutic composition comprising the same, and the use of these compositions to treat obesity or cardiometabolic disorders. In some aspects, the compositions comprise a miRNA agent and a targeting agent. In some embodiments, the targeting agent may be an aptamer, an exosome, or both an aptamer and an exosome.

### CROSS REFERENCE TO RELATED APPLICATIONS

This application claims priority to U.S. Application Ser. No. 61/679,461, filed on Aug. 3, 2012, U.S. Application Ser. No. 61/681,739, filed on Aug. 10, 2012, and U.S. Application Ser. No. 61/695,477, filed on Aug. 31, 2012, each of which hereby incorporated by reference in their entirety.

### BACKGROUND OF THE INVENTION

A. Field of the Invention

The invention generally concerns compositions comprising microRNAs (miRNAs) and targeting agents, as well as methods for delivering a therapeutic composition comprising the same, and the use of these compositions to treat obesity or cardiometabolic disorders.

B. Description of Related Art

Obesity has reached pandemic proportions globally, affecting all ages and socioeconomic groups. The World Health Organization estimated that in 2008, 1.5 billion adults aged 20 years and older were overweight and over 500 million men and women were obese. These numbers are estimated to increase to 2.16 billion overweight and 1.12 billion obese individuals by 2030. Obesity is the source of lost earnings, restricted activity days, absenteeism, lower productivity at work (presenteeism), reduced quality of life, permanent disability, significant morbidity and mortality, and shortened lifespan. The total annual economic cost of overweight and obesity in the United States and Canada caused by medical costs, excess mortality and disability was estimated to be about $300 billion in 2009. International studies on the economic costs of obesity have shown that they account for between 2% and 10% of total health care costs.

Obesity is the result of a chronic imbalance between energy intake and expenditure. This leads to storage of excess energy in adipocytes, which typically exhibit both hypertrophy (increase in cell size) and hyperplasia (increase in cell number or adipogenesis). The recent worsening of obesity is due to the combination of excessive consumption of energy-dense foods high in saturated fats and sugars, and reduced physical activity.

The current symptomatic medical treatments of obesity fail to achieve their long-term therapeutic goals, largely due to limited drug efficacy and patients' poor adherence with lifestyle changes and therapies. Several obesity drugs have been removed from the market for safety reasons and small molecules currently in development are struggling to gain regulatory approval because of their modest short-term efficacy and unknown safety profile. Presently, only restrictive and malabsorptive bariatric surgery can achieve significant long-term reduction of weight excess with some favorable cardiovascular benefits.

Accordingly, there is a need in the art for novel treatments for obesity.

### SUMMARY OF THE INVENTION

In some aspects, disclosed herein are compositions comprising a miRNA agent and a targeting agent. In some embodiments, the targeting agent may be an aptamer, an exosome, or both an aptamer and an exosome.

MicroRNAs (abbreviated miRNAs) are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression. In some embodiments, the miRNA agent is a miRNA or analog thereof. In some embodiments, the miRNA agent is thermogenic, adipogenic, or thermogenic and adipogenic. In some embodiments, the miRNA agent is thermogenic and adipogenic. In some embodiments, the miRNA agent is thermogenic. In some embodiments, the miRNA agent is a miRNA analog. In some embodiments, the miRNA analog is a miRNA agomir or a miRNA antagomir.

In some embodiments, the miRNA is (miRBase V.19 nomenclature) hsa-miR-515-3p, hsa-miR-141-3p, hsa-miR-20a-5p, hsa-miR-16-5p, hsa-miR-125a-5p, hsa-miR-let-7d-3p, hsa-miR-371a-3p, hsa-miR-195-5p, hsa-miR-106b-3p, hsa-miR-605, hsa-miR-217, hsa-miR-148a-3p, hsa-miR-19b-3p, hsa-miR-192-5p, hsa-miR-101-5p, hsa-miR-200b-3p, hsa-miR-487b, hsa-miR-1179, hsa-miR-223-3p, hsa-let-7a-3p, hsa-let-7a-5p, hsa-let-7b-3p, hsa-let-7b-5p, hsa-let-7c, hsa-let-7d-5p, hsa-let-7e-3p, hsa-let-7e-5p, hsa-let-7f-1-3p, hsa-let-7f-2-3p, hsa-let-7f-5p, hsa-let-7g-3p, hsa-let-7g-5p, hsa-let-7i-3p, hsa-let-7i-5p, hsa-miR-1, hsa-miR-100-3p, hsa-miR-100-5p, hsa-miR-101-3p, hsa-miR-103a-2-5p, hsa-miR-103a-3p, hsa-miR-103b, hsa-miR-105-5p, hsa-miR-106a-3p, hsa-miR-106a-5p, hsa-miR-106b-5p, hsa-miR-107, hsa-miR-10a-3p, hsa-miR-10a-5p, hsa-miR-10b-3p, hsa-miR-10b-5p, hsa-miR-1178-3p, hsa-miR-1180, hsa-miR-1181, hsa-miR-1182, hsa-miR-1183, hsa-miR-1184, hsa-miR-1185-5p, hsa-miR-1204, hsa-miR-1207, hsa-miR-1208, hsa-miR-122-3p, hsa-miR-122-5p, hsa-miR-1224, hsa-miR-1226, hsa-miR-1227-3p, hsa-miR-1228-5p, hsa-miR-1229-3p, hsa-miR-1231, hsa-miR-124-3p, hsa-miR-1245a, hsa-miR-1246, hsa-miR-1249, hsa-miR-125a-3p, hsa-miR-125b-1-3p, hsa-miR-125b-2-3p, hsa-miR-125b-5p, hsa-miR-1251, hsa-miR-1252, hsa-miR-1253, hsa-miR-1255a, hsa-miR-1255b-5p, hsa-miR-126-3p, hsa-miR-1260a, hsa-miR-1260b, hsa-miR-1262, hsa-miR-1263, hsa-miR-1265, hsa-miR-1268a, hsa-miR-127-3p, hsa-miR-1270, hsa-miR-1272, hsa-miR-1273a, hsa-miR-1275, hsa-miR-1276, hsa-miR-1277-3p, hsa-miR-1278, hsa-miR-128, hsa-miR-1285-3p, hsa-miR-1286, hsa-miR-1287, hsa-miR-129-2-3p, hsa-miR-1292-5p, hsa-miR-1293, hsa-miR-1301, hsa-miR-1302, hsa-miR-1303, hsa-miR-1305, hsa-miR-130a-3p, hsa-miR-130a-5p, hsa-miR-130b-3p, hsa-miR-130b-5p, hsa-miR-132-3p, hsa-miR-132-5p, hsa-miR-1321, hsa-miR-1323, hsa-miR-1324, hsa-miR-133a, hsa-miR-133b, hsa-miR-134, hsa-miR-135a-3p, hsa-miR-135a-5p, hsa-miR-135b-3p, hsa-miR-135b-5p, hsa-miR-136-3p, hsa-miR-136-5p, hsa-miR-137, hsa-miR-138-1-3p, hsa-miR-138-2-3p, hsa-miR-138-5p, hsa-miR-139-3p, hsa-miR-139-5p, hsa-miR-140-3p, hsa-miR-140-5p, hsa-miR-142-3p, hsa-miR-142-5p, hsa-miR-143-3p, hsa-miR-143-5p, hsa-miR-144-3p, hsa-miR-144-5p, hsa-miR-145-3p, hsa-miR-145-5p, hsa-miR-1468, hsa-miR-146a-5p, hsa-miR-146b-3p, hsa-miR-146b-5p, hsa-miR-147a, hsa-miR-147b, hsa-miR-148a-5p, hsa-miR-148b-3p, hsa-miR-148b-5p, hsa-miR-149-3p, hsa-miR-149-5p, hsa-miR-150-3p, hsa-miR-150-5p, hsa-miR-151a-3p, hsa-miR-151a-5p, hsa-miR-152, hsa-miR-153, hsa-miR-1538, hsa-miR-1539, hsa-miR-154-3p, hsa-miR-154-5p, hsa-miR-155-5p, hsa-miR-15a-3p, hsa-miR-15a-5p, hsa-miR-15b-3p, hsa-miR-15b-5p, hsa-miR-16-1-3p, hsa-miR-16-2-3p, hsa-miR-17-3p, hsa-miR-17-5p, hsa-miR-181a-2-3p, hsa-miR-181a-3p, hsa-miR-181a-5p, hsa-miR-181b-5p, hsa-miR-181c-3p, hsa-miR-181c-5p, hsa-miR-181d, hsa-miR-182-5p, hsa-miR-1827, hsa-miR-183-3p, hsa-miR-183-5p, hsa-miR-184, hsa-miR-185-3p, hsa-miR-185-5p, hsa-miR-186-3p, hsa-miR-186-5p, hsa-miR-187-3p, hsa-miR-187-5p, hsa-miR-188-5p, hsa-miR-18a-3p, hsa-miR-18a-5p, hsa-miR-18b-3p, hsa-miR-18b-5p, hsa-miR-190a, hsa-miR-190b, hsa-miR-191-3p, hsa-miR-191-5p, hsa-miR-1915-3p, hsa-miR-192-3p, hsa-miR-193a-3p, hsa-miR-193a-5p, hsa-miR-193b-3p, hsa-miR-193b-5p, hsa-miR-194-5p, hsa-miR-195-3p, hsa-miR-196a-3p, hsa-miR-196a-5p, hsa-miR-196b-5p, hsa-miR-197-3p, hsa-miR-198, hsa-miR-199a-3p, hsa-miR-199a-5p, hsa-miR-199b-3p, hsa-miR-199b-5p, hsa-miR-19a-3p, hsa-miR-19a-5p, hsa-miR-1909-3p, hsa-miR-1911-3p, hsa-miR-200a-3p, hsa-miR-200a-5p, hsa-miR-200b-5p, hsa-miR-200c-3p, hsa-miR-200c-5p, hsa-miR-202-3p, hsa-miR-202-5p, hsa-miR-203a, hsa-miR-203b-5p, hsa-miR-204-5p, hsa-miR-205-5p, hsa-miR-206, hsa-miR-208b, hsa-miR-20a-3p, hsa-miR-20b-3p, hsa-miR-20b-5p, hsa-miR-211-3p, hsa-miR-21-5p, hsa-miR-210, hsa-miR-211-5p, hsa-miR-2110, hsa-miR-212-3p, hsa-miR-214-3p, hsa-miR-214-5p, hsa-miR-215, hsa-miR-216a-5p, hsa-miR-218-1-3p, hsa-miR-218-5p, hsa-miR-219-1-3p, hsa-miR-219-5p, hsa-miR-22-3p, hsa-miR-22-5p, hsa-miR-221-3p, hsa-miR-221-5p, hsa-miR-222-3p, hsa-miR-222-5p, hsa-miR-223-5p, hsa-miR-224-3p, hsa-miR-224-5p, hsa-miR-2355-3p, hsa-miR-23a-3p, hsa-miR-23a-5p, hsa-miR-23b-3p, hsa-miR-23b-5p, hsa-miR-24-1-5p, hsa-miR-24-2-5p, hsa-miR-24-3p, hsa-miR-25-3p, hsa-miR-25-5p, hsa-miR-26a-2-3p, hsa-miR-26a-5p, hsa-miR-26b-3p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a-3p, hsa-miR-29a-5p, hsa-miR-29b-1-5p, hsa-miR-29b-2-5p, hsa-miR-29b-3p, hsa-miR-29c-3p, hsa-miR-29c-5p, hsa-miR-2909, hsa-miR-301a-3p, hsa-miR-301b, hsa-miR-302a-5p, hsa-miR-302b-5p, hsa-miR-302c-5p, hsa-miR-302d-3p, hsa-miR-302d-5p, hsa-miR-302f, hsa-miR-3064-5p, hsa-miR-3065-3p, hsa-miR-3065-5p, hsa-miR-3074-3p, hsa-miR-3074-5p, hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3120-3p, hsa-miR-3120-5p, hsa-miR-3184-5p, hsa-miR-32-3p, hsa-miR-32-5p, hsa-miR-320a, hsa-miR-320b, hsa-miR-320c, hsa-miR-323a-3p, hsa-miR-323b-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-5p, hsa-miR-346, hsa-miR-34a-3p, hsa-miR-34a-5p, hsa-miR-34b-3p, hsa-miR-34b-5p, hsa-miR-34c-3p, hsa-miR-34c-5p, hsa-miR-3591-3p, hsa-miR-361-3p, hsa-miR-361-5p, hsa-miR-3613-5p, hsa-miR-3615, hsa-miR-3619, hsa-miR-362-3p, hsa-miR-362-5p, hsa-miR-363-3p, hsa-miR-363-5p, hsa-mir-365a-3p, hsa-mir-365a-5p, hsa-mir-3653, hsa-miR-3656, hsa-miR-367-3p, hsa-miR-367-5p, hsa-miR-3676-3p, hsa-miR-369-3p, hsa-miR-369-5p, hsa-miR-370, hsa-miR-372, hsa-miR-373-3p, hsa-miR-373-5p, hsa-miR-374a-3p, hsa-miR-374a-5p, hsa-miR-374b-3p, hsa-miR-374b-5p, hsa-miR-375, hsa-miR-376a-2-5p, hsa-miR-376a-3p, hsa-miR-376a-5p, hsa-miR-376b-3p, hsa-miR-376c-3p, hsa-miR-377-3p, hsa-miR-377-5p, hsa-miR-378a-3p, hsa-miR-378a-5p, hsa-miR-378c, hsa-miR-378d, hsa-miR-379-3p, hsa-miR-379-5p, hsa-miR-380-3p, hsa-miR-380-5p, hsa-miR-381-3p, hsa-miR-382-3p, hsa-miR-382-5p, hsa-miR-383, hsa-miR-384, hsa-miR-3912, hsa-miR-3928, hsa-miR-409-3p, hsa-miR-409-5p, hsa-miR-410, hsa-miR-411-3p, hsa-miR-411-5p, hsa-miR-412, hsa-miR-421, hsa-miR-422a, hsa-miR-423-3p, hsa-miR-423-5p, hsa-miR-424-3p, hsa-miR-424-5p, hsa-miR-425-3p, hsa-miR-425-5p, hsa-miR-429, hsa-miR-4291, hsa-miR-431-5p, hsa-miR-432-5p, hsa-miR-433, hsa-miR-4421, hsa-miR-449a, hsa-miR-450a-5p, hsa-miR-450b-3p, hsa-miR-450b-5p, hsa-miR-4505, hsa-miR-4510, hsa-miR-4516, hsa-miR-451a, hsa-miR-452-3p, hsa-miR-452-5p, hsa-miR-4533, hsa-miR-4539, hsa-miR-454-3p, hsa-miR-454-5p, hsa-miR-455-3p, hsa-miR-455-5p, hsa-miR-4634, hsa-miR-4732-3p, hsa-miR-4732-5p, hsa-miR-4747-5p, hsa-miR-4792, hsa-miR-483-3p, hsa-miR-483-5p, hsa-miR-484, hsa-miR-485-5p, hsa-miR-486-3p, hsa-miR-486-5p, hsa-miR-489, hsa-miR-490, hsa-miR-491-3p, hsa-miR-491-5p, hsa-miR-492, hsa-miR-493-3p, hsa-miR-493-5p, hsa-miR-494, hsa-miR-495-3p, hsa-miR-496, hsa-miR-497-3p, hsa-miR-497-5p, hsa-miR-498, hsa-miR-499a-5p, hsa-miR-500a-3p, hsa-miR-501-3p, hsa-miR-501-5p, hsa-miR-502-3p, hsa-miR-502-5p, hsa-miR-503-5p, hsa-miR-504, hsa-miR-505-3p, hsa-miR-505-5p, hsa-miR-506-3p, hsa-miR-508-3p, hsa-miR-508-5p, hsa-miR-509-3p, hsa-miR-511, hsa-miR-512-5p, hsa-miR-513a-3p, hsa-miR-513a-5p, hsa-miR-513b, hsa-miR-514a-3p, hsa-miR-514a-5p, hsa-miR-515-5p, hsa-miR-516b-3p, hsa-miR-516b-5p, hsa-miR-517a-3p, hsa-miR-518a-3p, hsa-miR-518b, hsa-miR-518e-3p, hsa-miR-518e-5p, hsa-miR-518f-3p, hsa-miR-519a-5p, hsa-miR-519b-5p, hsa-miR-519c-3p, hsa-miR-519c-5p, hsa-miR-519d, hsa-miR-519e-5p, hsa-miR-520c-3p, hsa-miR-520e, hsa-miR-520f, hsa-miR-520g, hsa-miR-520h, hsa-miR-521, hsa-miR-522-5p, hsa-miR-523-5p, hsa-miR-525-3p, hsa-miR-532-3p, hsa-miR-532-5p, hsa-miR-539-5p, hsa-miR-541, hsa-miR-542-3p, hsa-miR-542-5p, hsa-miR-543, hsa-miR-545-3p, hsa-miR-545-5p, hsa-miR-548a-3p, hsa-miR-548d-3p, hsa-miR-548e, hsa-miR-548i, hsa-miR-548m, hsa-miR-549, hsa-miR-550a-3p, hsa-miR-550a-5p, hsa-miR-551b-3p, hsa-miR-551b-5p, hsa-miR-552, hsa-miR-553, hsa-miR-554, hsa-miR-557, hsa-miR-563, hsa-miR-564, hsa-miR-567, hsa-miR-569, hsa-miR-570-3p, hsa-miR-571, hsa-miR-572, hsa-miR-574-3p, hsa-miR-574-5p, hsa-miR-575, hsa-miR-576-3p, hsa-miR-576-5p, hsa-miR-577, hsa-miR-578, hsa-miR-580, hsa-miR-582-3p, hsa-miR-582-5p, hsa-miR-583, hsa-miR-584-5p, hsa-miR-585, hsa-miR-586, hsa-miR-589-3p, hsa-miR-589-5p, hsa-miR-590-3p, hsa-miR-590-5p, hsa-miR-593-3p, hsa-miR-593-5p, hsa-miR-595, hsa-miR-598, hsa-miR-601, hsa-miR-602, hsa-miR-603, hsa-miR-606, hsa-miR-608, hsa-miR-609, hsa-miR-611, hsa-miR-612, hsa-miR-613, hsa-miR-615-3p, hsa-miR-615-5p, hsa-miR-616-5p, hsa-miR-618, hsa-miR-619, hsa-miR-620, hsa-miR-623, hsa-miR-625-5p, hsa-miR-626, hsa-miR-627, hsa-miR-628-3p, hsa-miR-628-5p, hsa-miR-629-3p, hsa-miR-629-5p, hsa-miR-630, hsa-miR-631, hsa-miR-634, hsa-miR-635, hsa-miR-636, hsa-miR-638, hsa-miR-639, hsa-miR-641, hsa-miR-642a-3p, hsa-miR-642a-5p, hsa-miR-643, hsa-miR-645, hsa-miR-646, hsa-miR-647, hsa-miR-649, hsa-miR-650, hsa-miR-651, hsa-miR-652-3p, hsa-miR-653, hsa-miR-654-3p, hsa-miR-655, hsa-miR-656, hsa-miR-657, hsa-miR-658, hsa-miR-659-3p, hsa-miR-660-5p, hsa-miR-663a, hsa-miR-663b, hsa-miR-664a-3p, hsa-miR-664a-5p, hsa-miR-668, hsa-miR-671-5p, hsa-miR-675-3p, hsa-miR-675-5p, hsa-miR-7-1-3p, hsa-miR-7-2-3p, hsa-miR-7-5p, hsa-miR-708-3p, hsa-miR-708-5p, hsa-miR-718, hsa-miR-744-3p, hsa-miR-744-5p, hsa-miR-761, hsa-miR-765, hsa-miR-766, hsa-miR-767-3p, hsa-miR-769-3p, hsa-miR-769-5p, hsa-miR-770-5p, hsa-miR-874, hsa-miR-875, hsa-miR-876, hsa-miR-877-3p, hsa-miR-877-5p, hsa-miR-885-3p, hsa-miR-887, hsa-miR-888-3p, hsa-miR-889, hsa-miR-890, hsa-miR-891a, hsa-miR-891b, hsa-miR-9-3p, hsa-miR-9-5p, hsa-miR-92a-3p, hsa-miR-92b-3p, hsa-miR-92b-5p, hsa-miR-921, hsa-miR-922, hsa-miR-93-3p, hsa-miR-93-5p, hsa-miR-935, hsa-miR-940, hsa-miR-941, hsa-miR-942, hsa-miR-95, hsa-miR-96-3p, hsa-miR-96-5p, hsa-miR-98-5p, hsa-miR-99a-3p, hsa-miR-99a-5p, hsa-miR-99b-3p, and/or hsa-miR-99b-5p, or an analog thereof. The sequences of these miRNAs are well known in the art and may be found, for example, on the world wide web at mirbase.org.

In some currently preferred embodiments, the miRNA is hsa-miR-515-3p, hsa-miR-141-3p, hsa-miR-20a-5p, hsa-miR-16-5p, hsa-miR-125a-5p, hsa-miR-let-7d-3p, hsa-miR-371a-3p, hsa-miR-195-5p, hsa-miR-106b-3p, hsa-miR-605, hsa-miR-217, hsa-miR-148a-3p, hsa-miR-19b-3p, hsa-miR-192-5p, hsa-miR-101-5p, hsa-miR-200b-3p, hsa-miR-487b, hsa-miR-1179, hsa-miR-223-3p, hsa-miR-20b-5p, hsa-miR-27b-3p, hsa-miR-103a-3p, hsa-miR-22-3p, hsa-miR-34a-5p, hsa-miR-130b-3p, hsa-miR-132-3p, hsa-miR-181b-5p, hsa-miR-211-5p, hsa-miR-148b-3p, hsa-miR-17-5p, hsa-miR-182-5p, hsa-miR-27a-3p, hsa-miR-301a-3p, hsa-miR-204-5p, hsa-miR-143-3p, hsa-miR-1, hsa-miR-9-5p, hsa-miR-30a-5p, hsa-miR-138-5p, hsa-miR-382-5p, hsa-miR-106a-5p, hsa-miR-107, hsa-miR-135a-5p, hsa-miR-93-5p, hsa-miR-21-5p, hsa-miR-155-5p, hsa-miR-181a-5p, hsa-miR-519d, hsa-miR-96-5p, hsa-miR-212-3p, hsa-miR-29a-3p, hsa-miR-98-5p, hsa-let-7c, hsa-let-7d-5p, hsa-miR-183-5p, hsa-miR-19a-3p, hsa-miR-196a-5p, hsa-miR-30b-5p, hsa-miR-378a-3p, hsa-miR-302c-5p, hsa-miR-30e-5p, hsa-miR-130a-3p, hsa-let-7e-5p, hsa-miR-216a-5p, hsa-miR-450a-5p, hsa-miR-26b-5p, hsa-miR-181c-5p, hsa-miR-186-5p, hsa-miR-519c-3p, hsa-let-7b-5p, hsa-miR-10b-5p, hsa-miR-125b-5p, hsa-miR-134, hsa-miR-137, hsa-miR-150-5p, hsa-miR-153, hsa-miR-15b-5p, hsa-miR-196b-5p, hsa-miR-23a-3p, hsa-miR-29c-3p, hsa-miR-373-3p, hsa-miR-7-5p, hsa-miR-214-3p, hsa-miR-421, hsa-miR-15a-5p, hsa-miR-193b-3p, hsa-miR-194-5p, hsa-miR-30d-5p, hsa-miR-424-5p, hsa-miR-454-3p, hsa-miR-545-3p, hsa-miR-485-5p, hsa-miR-335-5p, hsa-miR-133a, hsa-miR-222-3p, hsa-miR-494, hsa-miR-498, hsa-miR-513a-5p, hsa-miR-92a-3p, hsa-miR-495-3p, hsa-miR-503-5p, hsa-miR-539-5p, hsa-miR-16-2-3p, hsa-miR-302b-5p, hsa-miR-425-3p, hsa-miR-99a-3p, hsa-let-7a-3p, hsa-miR-126-3p, hsa-miR-20a-3p, hsa-miR-499a-5p, hsa-let-7g-5p, hsa-miR-152, hsa-miR-26a-5p, hsa-miR-124-3p, hsa-miR-203a, hsa-miR-24-3p, hsa-miR-301b, hsa-miR-590-3p, hsa-miR-325, hsa-miR-552, hsa-miR-185-5p, hsa-miR-455-3p, hsa-miR-583, hsa-miR-122-5p, hsa-miR-1305, hsa-miR-139-5p, hsa-miR-146a-5p, hsa-miR-18a-5p, hsa-miR-18b-5p, hsa-miR-199b-5p, hsa-miR-340-5p, hsa-miR-34c-5p, hsa-miR-423-3p, hsa-miR-489, hsa-miR-520f, hsa-miR-520g, hsa-miR-668, hsa-let-7a-5p, hsa-let-7f-5p, hsa-miR-10a-3p, hsa-miR-135b-5p, hsa-miR-144-3p, hsa-miR-181d, hsa-miR-200c-3p, hsa-miR-218-5p, hsa-miR-23b-3p, hsa-miR-25-3p, hsa-miR-29b-3p, hsa-miR-383, hsa-miR-202-3p, hsa-miR-381-3p, hsa-miR-377-3p, hsa-miR-452-5p, hsa-miR-501-3p, hsa-miR-514a-3p, hsa-miR-654-3p, hsa-let-7b-3p, hsa-miR-125a-3p, hsa-miR-133b, hsa-miR-199a-3p, hsa-miR-30c-5p, hsa-miR-335-3p, hsa-miR-374a-5p, hsa-miR-410, hsa-miR-429, hsa-miR-497-5p, hsa-miR-513a-3p, hsa-miR-542-3p, hsa-miR-653, hsa-miR-122-3p, hsa-miR-1178-3p, hsa-miR-191-5p, hsa-miR-214-5p, hsa-miR-302d-5p, hsa-miR-572, hsa-miR-574-3p, hsa-miR-26a-2-3p, hsa-miR-611, hsa-let-7f-1-3p, hsa-let-7i-3p, hsa-miR-100-5p, hsa-miR-106b-5p, hsa-miR-132-5p, hsa-miR-135b-3p, hsa-miR-136-3p, hsa-miR-150-3p, hsa-miR-154-3p, hsa-miR-15a-3p, hsa-miR-15b-3p, hsa-miR-16-1-3p, hsa-miR-181a-2-3p, hsa-miR-181c-3p, hsa-miR-186-3p, hsa-miR-195-3p, hsa-miR-20b-3p, hsa-miR-223-5p, hsa-miR-224-3p, hsa-miR-24-1-5p, hsa-miR-24-2-5p, hsa-miR-27a-5p, hsa-miR-27b-5p, hsa-miR-29b-1-5p, hsa-miR-302a-5p, hsa-miR-3065-5p, hsa-miR-30d-3p, hsa-miR-34a-3p, hsa-miR-373-5p, hsa-miR-374a-3p, hsa-miR-376a-5p, hsa-miR-378a-5p, hsa-miR-424-3p, hsa-miR-451a, hsa-miR-452-3p, hsa-miR-493-5p, hsa-miR-500a-3p, hsa-miR-502-3p, hsa-miR-516b-3p, hsa-miR-518e-3p, hsa-miR-518f-3p, hsa-miR-519a-5p, hsa-miR-519b-5p, hsa-miR-521, hsa-miR-523-5p, hsa-miR-545-5p, hsa-miR-585, hsa-miR-7-2-3p, hsa-miR-93-3p, hsa-miR-96-3p, and/or hsa-miR-99b-3p, or an analog thereof.

In some currently even more preferred embodiments, the miRNA is hsa-miR-515-3p, hsa-miR-141-3p, hsa-miR-20a-5p, hsa-miR-16-5p, hsa-miR-125a-5p, hsa-miR-let-7d-3p, hsa-miR-371a-3p, hsa-miR-195-5p, hsa-miR-106b-3p, hsa-miR-605, hsa-miR-217, hsa-miR-148a-3p, hsa-miR-19b-3p, hsa-miR-192-5p, hsa-miR-101-5p, hsa-miR-200b-3p, hsa-miR-487b, hsa-miR-1179, and/or hsa-miR-223-3p, or an analog thereof.

In some embodiments, the composition comprises 1, 2, 3, 4, 5, 6, 7, 8, 9, 10, or more distinct miRNA agents or any range derivable therein.

In some embodiments, the targeting agent is capable of binding to a cell surface marker. In some embodiments, the cell surface marker is selected from the group consisting of: CD9 (tetraspan), CD10 (MME), CD13 (ANPEP), CD29 (β-1 integrin), CD36 (FAT), CD44 (hyaluronate), CD49d (α-4 integrin), CD54 (ICAM-1), CD55 (DAF), CD59, CD73 (SH3), CD90 (Thy1), CD91 (LPR1), CD105 (SH2, Endoglin), CD137, CD146 (Muc 18), CD166 (ALCAM), HLA-ABC, adiponectin, caveolin-1, caveolin-2, CD36 (FAT), CLH-22 (clathrin heavy chain chromosome 22), DPT (dermatopotin), FABP4 (adipocyte protein 2, ap2), SLC27A1 (FATP1), SLC27A2 (FATP2), GLUT4 (glucose transporter 4), perilipin 2, resistin, neprilysin (CD10), FAT (CD36), Thy-1 (CD90), low density lipoprotein receptor-related protein 1 (LRP1, CD91), caveolin-1, caveolin-2, fatty acid binding protein 4 (FABP4), cell surface glycoprotein MUC18 (CD146), activated leukocyte cell adhesion molecule (CD166) and Natriuretic peptide receptor A (NPR1). In some embodiments, the targeting agent does not bind to a hematopoietic lineage marker selected from the group consisting of: CD11b (α-M integrin), CD14, CD18, CD19, CD31, CD34, CD45 (LCA), CD79 alpha, c-kit (ABCG2), STRO-1, HLA II, Lin1, Ter119, and HLA-DR.

In some embodiments, the targeting agent is an aptamer, an exosome, or a combination of an aptamer and an exosome.

Aptamers are usually single-stranded, short molecules of RNA, DNA or a nucleic acid analog, that may adopt three-dimensional conformations complementary to a wide variety of target molecules.

Exosomes are small membrane vesicles of endocytic origin that are secreted by many cell types. For example, exosomes may have a diameter of about 30 to about 100 nm. They may be formed by inward budding of the late endosome leading to the formation of vesicle-containing multivesicular bodies (MVB) which then fuse with the plasma membrane to release exosomes into the extracellular environment. Though their exact composition and content depends on cell type and disease state, exosomes all share certain characteristics. In some embodiments, the composition further comprises a nanoparticle, such as an exosome, wherein the nanoparticle has a diameter of no more than 100 nm. In some embodiments, the nanoparticle has a diameter of equal to or between about 30 nm and about 100 nm. In some embodiments, the miRNA agent is encapsulated by the nanoparticle.

In some embodiments, the targeting agent is bound to the outside of the nanoparticle. In some embodiments,

In some embodiments, the miRNA agent is covalently coupled to the targeting agent. In some embodiments, the miRNA agent is non-covalently coupled to the targeting agent. In some embodiments, the miRNA agent is coupled to the targeting agent by a linker. In some embodiments, the linker is selected from the group consisting of: a polyalkylene glycol, polyethylene glycol, a dendrimer, a comb polymer, a biotinstreptavidin bridge, and a ribonucleic acid.

In some embodiments, the composition is an aptamir composition. In some embodiments, the aptamir composition is an adipocyte-specific aptamir composition. In some embodiments, the composition further comprises a pharmaceutically acceptable carrier.

Also disclosed herein are the use of the compositions disclosed herein to treat obesity or metabolic disorders in a subject. In some embodiments, the compositions disclosed herein may be for use as a medicament. In some embodiments, the compositions disclosed herein may be for use in treating obesity or metabolic disorders in a subject.

In some embodiments, disclosed herein are methods for delivering a therapeutic microRNA (miRNA) to a specific cell, tissue, or organ in a subject comprising administering any of the aptamir compositions disclosed herein to the subject. In some embodiments, the cell is an adipocyte or adipose tissue derived mesenchymal stem cell. In some embodiments, the miRNA agent modulates activity of at least one mitochondrial uncoupler. In some embodiments, the mitochondrial uncoupler is UCP1, UCP2, or UCP3. In some embodiments, the miRNA modulator directly binds to the mRNA or promoter region of at least one mitochondrial uncoupler. In some embodiments, the miRNA agent directly binds to the 5′UTR or coding sequence of the mRNA of at least one mitochondrial uncoupler.

In some embodiments, disclosed herein are methods for treating obesity or metabolic disorders in a subject, comprising administering the aptamir composition of any one of the compositions disclosed herein to the subject. In some embodiments, the composition may contain an effective amount of any one of the compositions disclosed herein. In some embodiments, the aptamir composition is delivered to a cell, tissue, or organ in the subject. In some embodiments, the cell is an adipocyte or adipose tissue derived mesenchymal stem cell. In some embodiments, the miRNA agent modulates activity of at least one mitochondrial uncoupler. In some embodiments, the mitochondrial uncoupler is UCP1, UCP2, or UCP3. In some embodiments, the miRNA modulator directly binds to the mRNA or promoter region of at least one mitochondrial uncoupler. In some embodiments, the miRNA agent directly binds to the 5′UTR or coding sequence of the mRNA of at least one mitochondrial uncoupler.

“Treatment” or “treating,” as used herein, is defined as the application or administration of a therapeutic agent (e.g., a miRNA agent or vector or transgene encoding same) to a patient, or application or administration of a therapeutic agent to an isolated tissue or cell line from a patient, and includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Effective amount” or “therapeutically effective amount” or “pharmaceutically effective amount” means that amount which, when administered to a subject or patient for treating a disease, is sufficient to effect such treatment for the disease. In some embodiments, the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, or 100 mg/kg (or any range derivable therein).

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient.

In some embodiments, the compounds described herein are comprised in a pharmaceutical composition. In further embodiments, the compounds described herein and optional one or more additional active agents, can be optionally combined with one or more pharmaceutically acceptable excipients and formulated for administration via epidural, introperitoneal, intramuscular, cutaneous, subcutaneous or intravenous injection. In some aspects, the compounds or the composition is administered by aerosol, infusion, or topical, nasal, oral, anal, ocular, or otic delivery. In further embodiments, the pharmaceutical composition is formulated for controlled release.

In additional embodiments, there are pharmaceutical compositions comprising the compositions discussed herein. Such a composition may or may not contain additional active ingredients. In certain embodiments, there is a pharmaceutical composition consisting essentially of a composition discussed herein. It is contemplated that the composition may contain non-active ingredients. Other aspects are directed to pharmaceutical compositions comprising an effective amount of a composition disclosed herein and a pharmaceutically acceptable carrier.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

The methods for producing the compounds described herein are not limited to the exemplary methods described herein. The compounds may be synthesized by any suitable method known in the art and it will be obvious to those skilled in the art that various adaptations, changes, modifications, substitutions, deletions or additions of procedures may be made without departing from the spirit and scope of the invention.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 illustrates a schematic representation of the in vitro aptamer selection (SELEX™) process from pools of random sequence oligonucleotides.

FIG. 2 illustrates a schematic representation of the Cell-SELEX process, a variation of the SELEX™ process that uses live intact cells.

FIG. 3 illustrates a schematic representation showing various modifications that can be made to an aptamer to increase its stability and functionality, such as one or more modifications at the sugar, base, or internucleotide linkage.

FIG. 4 illustrates a schematic representation of an exosome loaded with miRNAs.

FIG. 5 illustrates a schematic representation of a miRNA analog-loaded exosome whose envelope is studded with targeting aptamers.

FIG. 6 illustrates a schematic representation of an alternative embodiment of an exosome-based aptamir (so-called exomir) where the targeting aptamers at the surface of the exosome are coupled to anchoring proteins like Lamp2 or CD63.

FIG. 7 illustrates a schematic representation of the fate of an exomir entering a target cell.

FIG. 8 illustrates the results of a FACS experiment assessing binding of selected fluorescent aptamers to human hepatocytes and adipocytes. The top panel graphs are hepatocytes and the bottom panel graphs are adipocytes.

FIG. 9 illustrates the effect of several miRNA mimics on the expression level of the human UCP1 mRNA two weeks after a single transfection of mature human subcutaneous adipocytes with one of these mimics. The control condition (maintenance medium) is set at 100% and the PPARG agonist rosiglitazone is included as a positive control.

### DETAILED DESCRIPTION OF THE INVENTION

MicroRNAs (miRNAs) are attractive drug candidates for regulating cell fate decisions and improving complex diseases because the simultaneous modulation of many target genes by a single miRNA may provide effective therapies of multifactorial diseases like obesity. miRNA-based therapies offer several advantages over classical small molecules, which should translate into shorter and less expensive drug development times. Several miRNA agonists (agomirs) and antagonists (antagomirs) are currently developed to treat various human diseases, including cardiometabolic disorders.

miRNAs are extensive regulators of adipocyte differentiation, development and function and are viable therapeutic agents for obesity. A targeted intracellular delivery of miRNAs to specific cells or tissues should enhance their efficacy and safety. Nucleic acid-based aptamers and/or exosomes targeting cell surface molecules/receptors are promising delivery vehicles to target a distinct disease or tissue in a cell-type specific manner.

Thermogenic miRNA modulators are highly attractive as a therapy for obesity as they allow for the reduction of body fat in a subject without the need to adjust their caloric intake through dieting, modify their physical activity or undergo bariatric surgery.

Moreover, such modulators open the possibility of a pharmacologically driven switch from energy-storing and lipid-filled white adipose tissue (WAT) to energy-consuming brown adipose tissue (BAT). In exemplary embodiments, the thermogenic miRNA modulator is capable of modulating an intracellular thermogenic regulator (e.g., a mitochondrial uncoupler, such as Thermogenin or Uncoupling Protein 1 (UCP1), Uncoupling Protein 2 (UCP2), or Uncoupling Protein 3 (UCP3). Accordingly, by facilitating targeted intracellular delivery to specific target cells or tissue, the targeted compositions of the invention enhance the safety and efficacy of the miRNA modulator.

### A. COMPOSITIONS COMPRISING ADIPOCYTE-SPECIFIC MIRNA MODULATORS

In some aspects, the present invention relates to compositions for modulating miRNAs. The compositions of the invention may comprise target miRNA modulators in which one or more miRNA modulator elements (e.g., any of the miRNA modulator agents or miRNA agents described herein) are combined with one or more carrier/targeting elements (e.g., any of the targeting agents described herein) to enhance specific cellular uptake, cellular distribution, and/or cellular activity of the miRNA modulator or miRNA analog. As used herein, the term “miRNA analog” refers to an oligonucleotide or oligonucleotide mimetic or inhibitor that directly or indirectly reprograms mesenchymal stem cells (ATMSCs) or white adipocytes (WAT) to become brown adipocytes (BAT). miRNA analogs can act on a target gene or an activator or repressor of a target gene, or on a target miRNA that directly or indirectly modulates the activity of a thermogenic regulator (e.g., a mitochondrial uncoupler or an activator or repressor thereof). As used herein, the term “mitochondrial uncoupler” refers to a protein (or the encoding nucleic acid) that can dissipate of the mitochondrial inner membrane proton gradient, thereby preventing the synthesis of ATP in the mitochondrion by oxidative phosphorylation. Exemplary mitochondrial uncouplers include UCP1, UCP2 and UCP3.

In some embodiments, the miRNA analog is linked (covalently or non-covalently) to the targeting agent (e.g., aptamer). In other embodiments, the miRNA modulator is admixed with the targeting element in a single composition (e.g., in a exosome or nanoparticle formulation).

1. AptamiR Compositions

In certain exemplary embodiments, the miRNA modulator is combined with an aptamer to create an “AptamiR” composition. There are many different ways to combine an aptamer and miRNA analog(s) to create an aptamir. They include, for example, aptamer-miRNA analog chimeras, aptamer-splice-switching oligonucleotide chimeras, and aptamer conjugated to nanoparticles or exosomes containing the miRNA analog(s).

Aptamers are usually single-stranded, short molecules of RNA, DNA or a nucleic acid analog, that may adopt three-dimensional conformations complementary to a wide variety of target molecules. Methods of constructing and determining the binding characteristics of aptamers are well known in the art. For example, such techniques are described in U.S. Pat. Nos. 5,582,981, 5,595,877 and 5,637,459, each incorporated herein by reference.

Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred.

Aptamers need to contain the sequence that confers binding specificity, but may be extended with flanking regions and otherwise derivatized. In preferred embodiments, the target-binding sequences of aptamers may be flanked by primer-binding sequences, facilitating the amplification of the aptamers by PCR or other amplification techniques. In a further embodiment, the flanking sequence may comprise a specific sequence that preferentially recognizes or binds a moiety to enhance the immobilization of the aptamer to a substrate.

Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

Methods for preparation and screening of aptamers that bind to particular targets of interest are well known, for example U.S. Pat. No. 5,475,096 and U.S. Pat. No. 5,270,163, each incorporated by reference. The technique generally involves selection from a mixture of candidate aptamers and step-wise iterations of binding, separation of bound from unbound aptamers and amplification. Because only a small number of sequences (possibly only one molecule of aptamer) corresponding to the highest affinity aptamers exist in the mixture, it is generally desirable to set the partitioning criteria so that a significant amount of aptamers in the mixture (approximately 5-50%) are retained during separation. Each cycle results in an enrichment of aptamers with high affinity for the target. Repetition for between six to twenty selection and amplification cycles may be used to generate aptamers that bind with high affinity and specificity to the target. Aptamers may be selected to specifically bind to adipocytes and related cells.

In one embodiment, an aptamiR composition comprises an aptamer that is directly linked or fused to miRNA modulators. Such aptamiRs are entirely chemically synthesized, which provides more control over the composition of the conjugate. For instance, the stoichiometry (ratio of miRNA analog per aptamer) and site of attachment can be precisely defined. The linkage portion of the conjugate presents a plurality (2 or more) of nucleophilic and/or electrophilic moieties that serve as the reactive attachment point for the aptamers and miRNA analogs. In addition, the aptamir may further comprise a linker between the aptamer and the miRNA analog. In some embodiments, the linker is a polyalkylene glycol, particularly a polyethylene glycol. In other embodiments, the linker is a exosome, dendrimer, or comb polymer. Other linkers can mediate the conjugation between the aptamer and the miRNA analog, including a biotinstreptavidin bridge, or a ribonucleic acid. Exemplary non-covalent linkers include linkers formed by base pairing a single stranded portion or overhang of the miRNA element and a complementary single-stranded portion or overhang of the aptamer element.

2. ExomiR Composition

In a further particular embodiment, an aptamer is combined with a miRNA analog in the form of a carrier-based aptamiR, described as an “ExomiR”. Exemplary carriers include nanoparticles or exosomes. Nanoparticle approaches have several functional advantages, including, for example, cellular uptake, the ability to cross membranes, and triggered nanoparticle disassembly. In some embodiments, the miRNA agent is encapsulated within the nanoparticle exosome. In some embodiments, the targeting agent is bound to the outside of the nanoparticle. The nano particle is no more than 100 nm in diameter.

In a particular embodiment, an aptamer is anchored at the surface of an exosome containing a load of miRNA analogs, i.e., an ExomiR composition. Exosomes are spherical nanostructures made of a lipid bilayer that can be loaded with pharmaceuticals, such as miRNAs.

Exosomes were first described as a means for reticulocytes to selectively discard transferrin receptors as they matured into erythrocytes (Johnstone, et al., 1987). For a long time thereafter, they were seen as mere ‘garbage cans’ for the removal of unwanted cellular components. However, since the discovery that B cells shed exosomes containing antigen-specific MHC II capable of inducing T cell responses (Raposo, et al., 1996), an abundance of exosome research has revealed that these small vesicles are involved in a multitude of functions, both physiological and pathological.

Exosomes are small membrane vesicles of endocytic origin that are secreted by many cell types. For example, exosomes may have a diameter of about 30 to about 100 nm. They may be formed by inward budding of the late endosome leading to the formation of vesicle-containing multivesicular bodies (MVB) which then fuse with the plasma membrane to release exosomes into the extracellular environment. Though their exact composition and content depends on cell type and disease state, exosomes all share certain structural characteristics.

In certain aspects, the exosomes may be purified by ultracentrifugation in a sucrose gradient, then identified by the presence of marker proteins such as Alix and CD63 (Schorey & Bhatnagar, 2008) or enrichment of tetraspanins and heat shock protein 70 (Lee, et al., 2011), all of which are specifically expressed in exosomes.

Exosomes also have the potential for directional homing to specific target cells, dependent on the physical properties of their membranes. Their effect can be local, regional or systemic. Exosomes do not contain a random sampling of their parent cell's cytoplasm, but are enriched in specific mRNA, miRNA, and proteins (Bobrie, et al., 2011). This cargo is protected from degradation by proteases and RNases while the vesicle is in the interstitial space, and retains bioactivity once taken up by a recipient cell. In this way, they facilitate the transfer of interactive signaling and enzymatic activities that would otherwise be restricted to individual cells based on gene expression (Lee, et al., 2011). For example, Skog and coworkers show that mRNA for a reporter protein can be incorporated into exosomes, transferred to a recipient cell, and translated (Skog, et al., 2008).

Selective purification or enrichment of physiologically active subpopulations of exosomes may be achieved via several procedures. In certain embodiments, effective exosomes may be concentrated to an enriched sample via use of specific surface protein markers and related separation techniques. In other embodiments, effective exosomes may be harvested from enriched primary cells cultures identified as capable of producing the effective exosomes. In further embodiments, based on screening procedures used to identify candidate effective exosome species, other exosomes may be fabricated using molecular engineering strategies designed to selectively produce exosomes containing the target (i.e., postulated) therapeutic molecular species. The latter may be confirmed by application of exosomes containing fabricated species to naïve cultures, where the desired effect (e.g., increased myelination) may be verified.

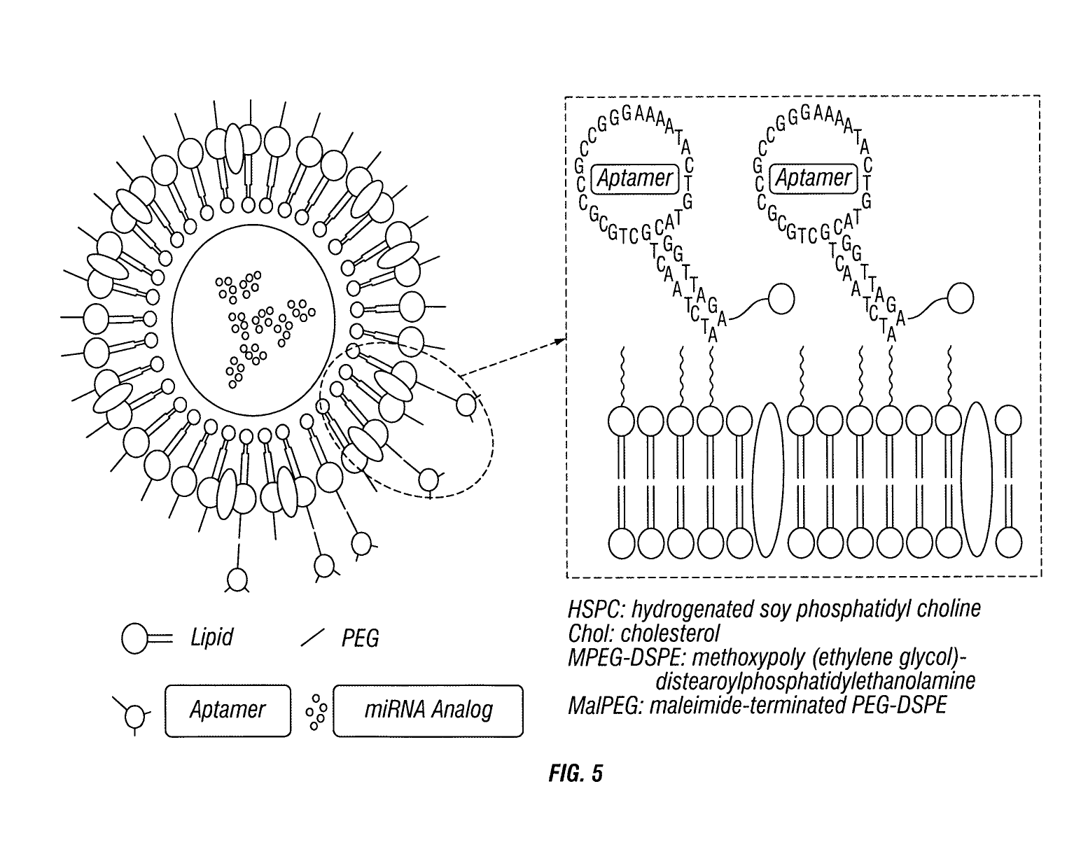

The exosome surface can be loaded with different substances, such as polyethylene glycol (extending their systemic half life) or molecular recognition elements like aptamers for specific binding and fusion to targeted cells. For example, aptamer-modified exosomes have been developed, with each exosome displaying approximately 250 aptamers tethered to its surface to facilitate target binding (FIG. 5). Glycosylphosphatidylinositol-anchored adiposomes transfer antilipolytic compounds from large donor adipocytes to small acceptor adipocytes.

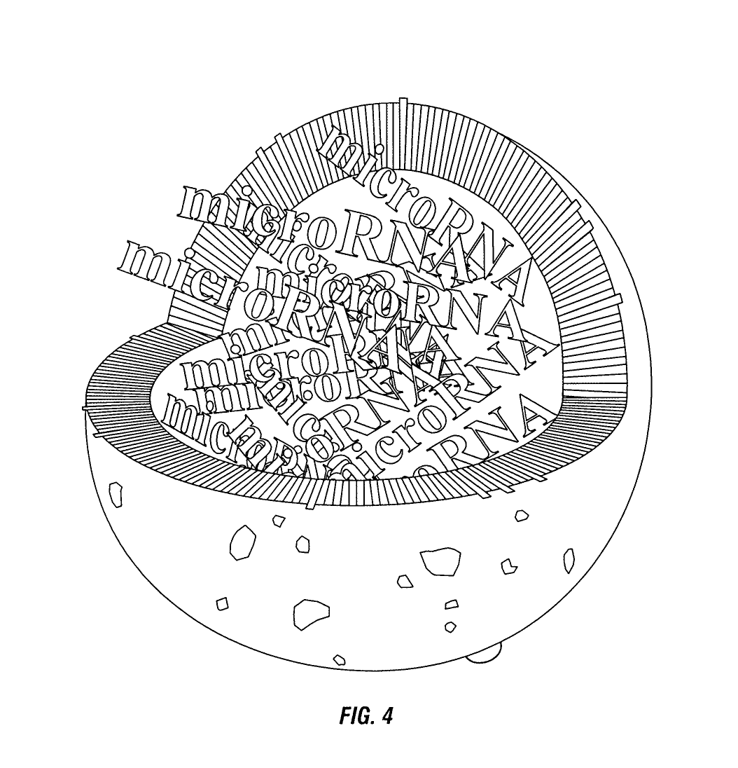

In a preferred embodiment, exosomes are created to encapsulate miRNA analog(s) and display at their surface aptamers that specifically bind with high affinity and specificity to molecules (e.g. lipid transporters) highly expressed at the surface of adipocytes and ATMSCs. See FIG. 4. The fusion of the exosomes with the targeted cells causes the release of the miRNA analog(s) into the cell cytoplasm, which then alter a specific intra-cellular pathway. Alternatively, stable thioaptamers may be inserted at the surface of exosomes to guide delivery of the exosome miRNA analog(s) load to targeted ATMSCs and adipocytes. See FIG. 5.

Exosomes are naturally occurring biological membrane vesicles measuring 30 to 100 nm that are secreted by most cells. They display surface receptors/molecules for cell targeting, adhesion and fusion, and also contain lipids, proteins, mRNAs and miRNAs. Exosomes are involved in the transport of genetic material while preserving it from circulating nucleases, the modulation of the immune system and cell-to-cell communications. Exosomes and their cargo load can efficiently cross barriers such as the skin, the intestinal mucosae and the blood-brain barrier. Exosomes are not recognized by macrophages, are not subject to attack by opsonins, complement factors, coagulation factors or antibodies in the circulation. They do not trigger innate immune reactions and are not cytotoxic.

Being natural shuttles of functional miRNAs, exosomes represent novel nano-scale delivery vehicles of miRNA analogs directly into the cytosol of target cells, as an alternative to liposomes. For instance, human breast milk exosomes contain 602 unique mature miRNAs which can be transferred from the mother to her infant (Zhou, 2012).

Exosomes released by “donor adipocytes”, which harbor glycosylphosphatidylinositol (GPI)-anchored proteins like Gce-1 and CD73, can specifically transfer mRNAs and miRNAs to “acceptor adipocytes” where they modulate lipogenesis and cell size (Müller, 2011). Such paracrine and endocrine regulation of adipocyte functions and size represents a novel therapeutic approach to metabolic diseases such as obesity and metabolic syndrome.

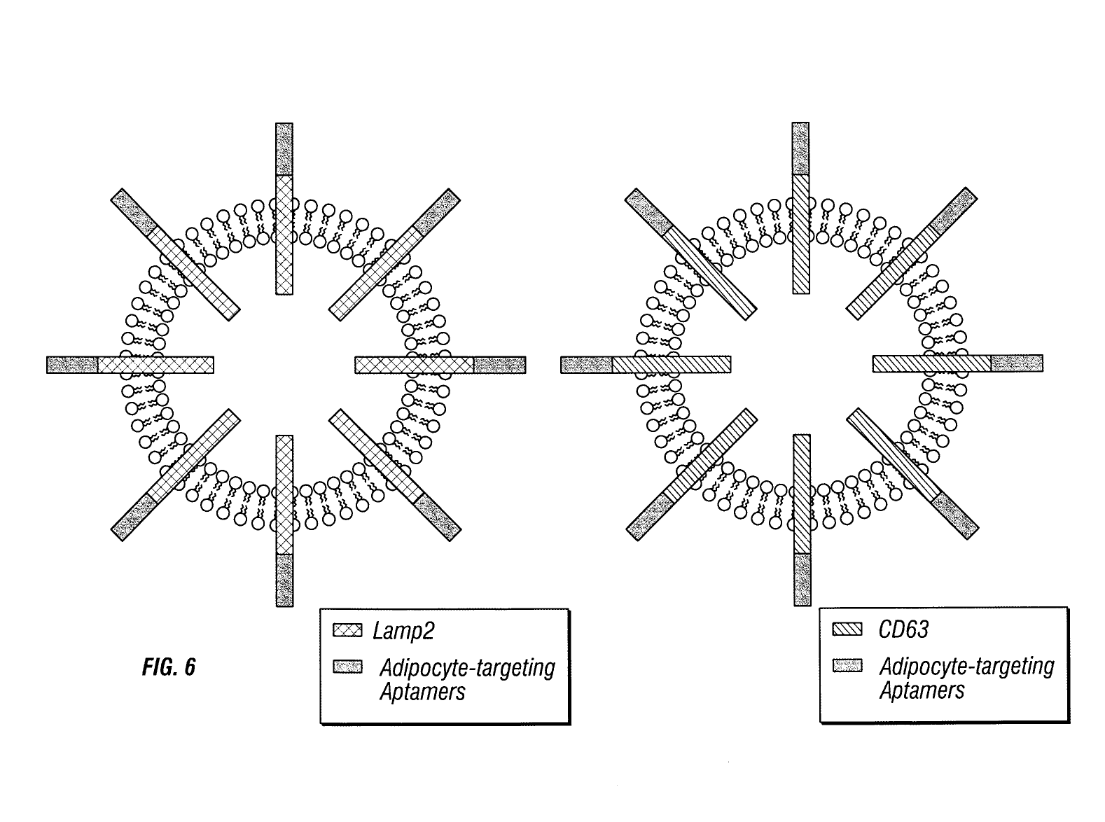

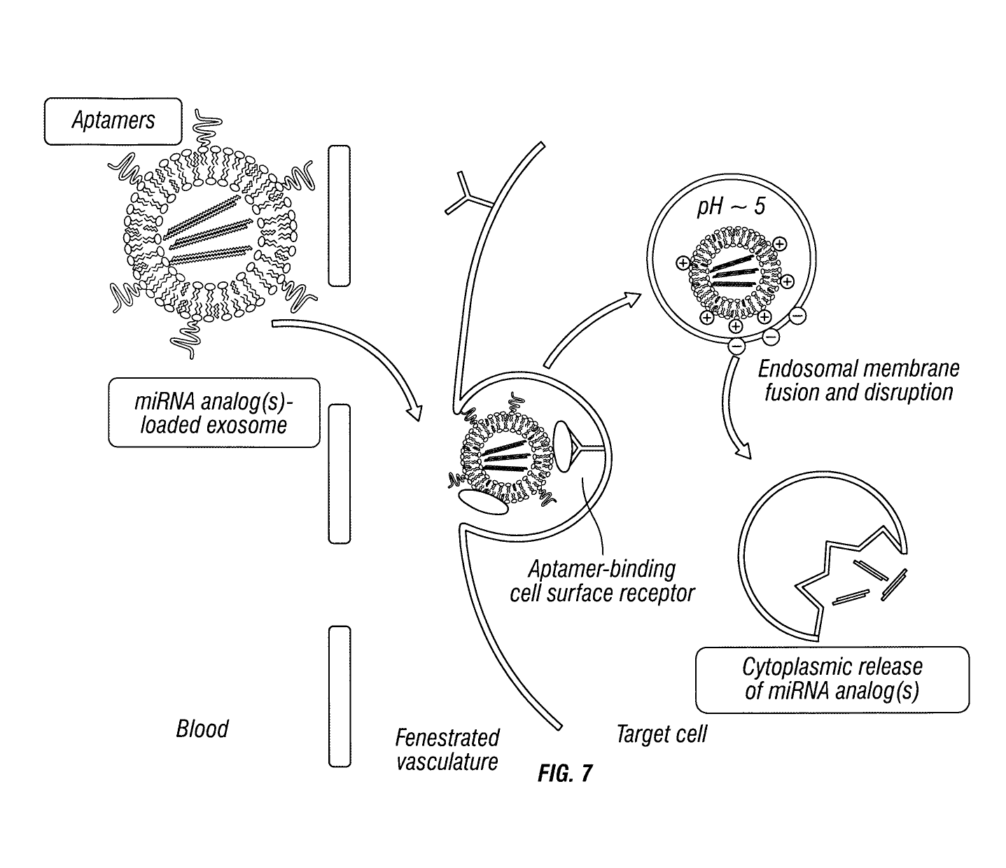

Such carrier-based aptamir compositions have the capability of delivering a cargo of multiple miRNA modulators to the target cell in a single carrier. To accomplish targeting and accumulation, the carriers are formulated to present the targeting element on their external surface so they can react/bind with selected cell surface antigens or receptors on the adipose target cell. See FIG. 6. As an example, carriers may be created to encapsulate miRNA modulators while displaying at their surface aptamers that specifically bind with high affinity and specificity to molecules (e.g. lipid transporters) highly expressed at the surface of adipocytes and ATMSCs. The internalized exosomes release inside the cell cytoplasm their miRNA analog(s) load, which alters a specific intra-cellular pathway. See FIG. 7.

In one embodiment, the carrier is an exosome. Exosomes, which originate from late endosomes, are naturally occurring nanoparticles that are specifically loaded with proteins, mRNAs, or miRNAs, and are secreted endogenously by cells. Exosomes are released from host cells, are not cytotoxic, and can transfer information to specific cells based on their composition and the substance in/on the exosome. Because exosomes are particles of approximately 30-100 nm in diameter, the exosomes evade clearance by the mononuclear phagocyte system (which clears circulating particles >100 nm in size), and are very efficiently delivered to target tissues.

Moreover, synthetic exosomes may offer several advantages over other carriers. For example, they may deliver their cargo directly into the cytosol, while their inertness avoids immune reactions and clearance in the extracellular environment. The structural constituents of exosomes may include small molecules responsible for processes like signal transduction, membrane transport, antigen presentation, targeting/adhesion, among many others.

### B. MIRNA MODULATOR ELEMENTS

In some embodiments, the present invention relates to compositions comprising a miRNA agent. In some embodiments, the miRNA agent is a miRNA, or an agomir or antagomir thereof. As used herein, the term “miRNA” refers to a single-stranded RNA molecule (or a synthetic derivative thereof), which is capable of binding to a target gene (either the mRNA or the DNA) and regulating expression of that gene. In certain embodiments, the miRNA is naturally expressed in an organism. In some embodiments, the miRNA agent is a thermogenic miRNA or analog thereof.

MicroRNAs (abbreviated miRNAs) are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression.

There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nt overhang at the 3′ end.

The cleavage product, the premature miRNA (pre-miRNA) is about 60 to about 110 nt long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length.

MicroRNAs function by engaging in base pairing (perfect or imperfect) with specific sequences in their target genes' messages (mRNA). The miRNA degrades or represses translation of the mRNA, causing the target genes' expression to be post-transcriptionally down-regulated, repressed, or silenced. In animals, miRNAs do not necessarily have perfect homologies to their target sites, and partial homologies lead to translational repression, whereas in plants, where miRNAs tend to show complete homologies to the target sites, degradation of the message (mRNA) prevails.

MicroRNAs are widely distributed in the genome, dominate gene regulation, and actively participate in many physiological and pathological processes. For example, the regulatory modality of certain miRNAs is found to control cell proliferation, differentiation, and apoptosis; and abnormal miRNA profiles are associated with oncogenesis. Additionally, it is suggested that viral infection causes an increase in miRNAs targeted to silence “pro-cell survival” genes, and a decrease in miRNAs repressing genes associated with apoptosis (programmed cell death), thus tilting the balance towards gaining apoptosis signaling.

In some embodiments, the miRNA is (miRBase V.19 nomenclature) hsa-let-7d-3p, hsa-miR-101-5p, hsa-miR-106b-3p, hsa-miR-1179, hsa-miR-125a-5p, hsa-miR-141-3p, hsa-miR-148a-3p, hsa-miR-16-5p, hsa-miR-192-5p, hsa-miR-195-5p, hsa-miR-19b-3p, hsa-miR-20a-5p, hsa-miR-200b-3p, hsa-miR-217, hsa-miR-223-3p, hsa-miR-371a-3p, hsa-miR-487b, hsa-miR-515-3p, hsa-miR-605, hsa-let-7a-3p, hsa-let-7a-5p, hsa-let-7b-3p, hsa-let-7b-5p, hsa-let-7c, hsa-let-7d-5p, hsa-let-7e-3p, hsa-let-7e-5p, hsa-let-7f-1-3p, hsa-let-7f-2-3p, hsa-let-7f-5p, hsa-let-7g-3p, hsa-let-7g-5p, hsa-let-7i-3p, hsa-let-7i-5p, hsa-miR-1, hsa-miR-100-3p, hsa-miR-100-5p, hsa-miR-101-3p, hsa-miR-103a-2-5p, hsa-miR-103a-3p, hsa-miR-103b, hsa-miR-105-5p, hsa-miR-106a-3p, hsa-miR-106a-5p, hsa-miR-106b-5p, hsa-miR-107, hsa-miR-10a-3p, hsa-miR-10a-5p, hsa-miR-10b-3p, hsa-miR-10b-5p, hsa-miR-1178-3p, hsa-miR-1180, hsa-miR-1181, hsa-miR-1182, hsa-miR-1183, hsa-miR-1184, hsa-miR-1185-5p, hsa-miR-1204, hsa-miR-1207, hsa-miR-1208, hsa-miR-122-3p, hsa-miR-122-5p, hsa-miR-1224, hsa-miR-1226, hsa-miR-1227-3p, hsa-miR-1228-5p, hsa-miR-1229-3p, hsa-miR-1231, hsa-miR-124-3p, hsa-miR-1245a, hsa-miR-1246, hsa-miR-1249, hsa-miR-125a-3p, hsa-miR-125b-1-3p, hsa-miR-125b-2-3p, hsa-miR-125b-5p, hsa-miR-1251, hsa-miR-1252, hsa-miR-1253, hsa-miR-1255a, hsa-miR-1255b-5p, hsa-miR-126-3p, hsa-miR-1260a, hsa-miR-1260b, hsa-miR-1262, hsa-miR-1263, hsa-miR-1265, hsa-miR-1268a, hsa-miR-127-3p, hsa-miR-1270, hsa-miR-1272, hsa-miR-1273a, hsa-miR-1275, hsa-miR-1276, hsa-miR-1277-3p, hsa-miR-1278, hsa-miR-128, hsa-miR-1285-3p, hsa-miR-1286, hsa-miR-1287, hsa-miR-129-2-3p, hsa-miR-1292-5p, hsa-miR-1293, hsa-miR-1301, hsa-miR-1302, hsa-miR-1303, hsa-miR-1305, hsa-miR-130a-3p, hsa-miR-130a-5p, hsa-miR-130b-3p, hsa-miR-130b-5p, hsa-miR-132-3p, hsa-miR-132-5p, hsa-miR-1321, hsa-miR-1323, hsa-miR-1324, hsa-miR-133a, hsa-miR-133b, hsa-miR-134, hsa-miR-135a-3p, hsa-miR-135a-5p, hsa-miR-135b-3p, hsa-miR-135b-5p, hsa-miR-136-3p, hsa-miR-136-5p, hsa-miR-137, hsa-miR-138-1-3p, hsa-miR-138-2-3p, hsa-miR-138-5p, hsa-miR-139-3p, hsa-miR-139-5p, hsa-miR-140-3p, hsa-miR-140-5p, hsa-miR-142-3p, hsa-miR-142-5p, hsa-miR-143-3p, hsa-miR-143-5p, hsa-miR-144-3p, hsa-miR-144-5p, hsa-miR-145-3p, hsa-miR-145-5p, hsa-miR-1468, hsa-miR-146a-5p, hsa-miR-146b-3p, hsa-miR-146b-5p, hsa-miR-147a, hsa-miR-147b, hsa-miR-148a-5p, hsa-miR-148b-3p, hsa-miR-148b-5p, hsa-miR-149-3p, hsa-miR-149-5p, hsa-miR-150-3p, hsa-miR-150-5p, hsa-miR-151a-3p, hsa-miR-151a-5p, hsa-miR-152, hsa-miR-153, hsa-miR-1538, hsa-miR-1539, hsa-miR-154-3p, hsa-miR-154-5p, hsa-miR-155-5p, hsa-miR-15a-3p, hsa-miR-15a-5p, hsa-miR-15b-3p, hsa-miR-15b-5p, hsa-miR-16-1-3p, hsa-miR-16-2-3p, hsa-miR-17-3p, hsa-miR-17-5p, hsa-miR-181a-2-3p, hsa-miR-181a-3p, hsa-miR-181a-5p, hsa-miR-181b-5p, hsa-miR-181c-3p, hsa-miR-181c-5p, hsa-miR-181d, hsa-miR-182-5p, hsa-miR-1827, hsa-miR-183-3p, hsa-miR-183-5p, hsa-miR-184, hsa-miR-185-3p, hsa-miR-185-5p, hsa-miR-186-3p, hsa-miR-186-5p, hsa-miR-187-3p, hsa-miR-187-5p, hsa-miR-188-5p, hsa-miR-18a-3p, hsa-miR-18a-5p, hsa-miR-18b-3p, hsa-miR-18b-5p, hsa-miR-190a, hsa-miR-190b, hsa-miR-191-3p, hsa-miR-191-5p, hsa-miR-1915-3p, hsa-miR-192-3p, hsa-miR-193a-3p, hsa-miR-193a-5p, hsa-miR-193b-3p, hsa-miR-193b-5p, hsa-miR-194-5p, hsa-miR-195-3p, hsa-miR-196a-3p, hsa-miR-196a-5p, hsa-miR-196b-5p, hsa-miR-197-3p, hsa-miR-198, hsa-miR-199a-3p, hsa-miR-199a-5p, hsa-miR-199b-3p, hsa-miR-199b-5p, hsa-miR-19a-3p, hsa-miR-19a-5p, hsa-miR-1909-3p, hsa-miR-1911-3p, hsa-miR-200a-3p, hsa-miR-200a-5p, hsa-miR-200b-5p, hsa-miR-200c-3p, hsa-miR-200c-5p, hsa-miR-202-3p, hsa-miR-202-5p, hsa-miR-203a, hsa-miR-203b-5p, hsa-miR-204-5p, hsa-miR-205-5p, hsa-miR-206, hsa-miR-208b, hsa-miR-20a-3p, hsa-miR-20b-3p, hsa-miR-20b-5p, hsa-miR-21-3p, hsa-miR-21-5p, hsa-miR-210, hsa-miR-211-5p, hsa-miR-2110, hsa-miR-212-3p, hsa-miR-214-3p, hsa-miR-214-5p, hsa-miR-215, hsa-miR-216a-5p, hsa-miR-218-1-3p, hsa-miR-218-5p, hsa-miR-219-1-3p, hsa-miR-219-5p, hsa-miR-22-3p, hsa-miR-22-5p, hsa-miR-221-3p, hsa-miR-221-5p, hsa-miR-222-3p, hsa-miR-222-5p, hsa-miR-223-5p, hsa-miR-224-3p, hsa-miR-224-5p, hsa-miR-2355-3p, hsa-miR-23a-3p, hsa-miR-23a-5p, hsa-miR-23b-3p, hsa-miR-23b-5p, hsa-miR-24-1-5p, hsa-miR-24-2-5p, hsa-miR-24-3p, hsa-miR-25-3p, hsa-miR-25-5p, hsa-miR-26a-2-3p, hsa-miR-26a-5p, hsa-miR-26b-3p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a-3p, hsa-miR-29a-5p, hsa-miR-29b-1-5p, hsa-miR-29b-2-5p, hsa-miR-29b-3p, hsa-miR-29c-3p, hsa-miR-29c-5p, hsa-miR-2909, hsa-miR-301a-3p, hsa-miR-301b, hsa-miR-302a-5p, hsa-miR-302b-5p, hsa-miR-302c-5p, hsa-miR-302d-3p, hsa-miR-302d-5p, hsa-miR-302f, hsa-miR-3064-5p, hsa-miR-3065-3p, hsa-miR-3065-5p, hsa-miR-3074-3p, hsa-miR-3074-5p, hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3120-3p, hsa-miR-3120-5p, hsa-miR-3184-5p, hsa-miR-32-3p, hsa-miR-32-5p, hsa-miR-320a, hsa-miR-320b, hsa-miR-320c, hsa-miR-323a-3p, hsa-miR-323b-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-5p, hsa-miR-346, hsa-miR-34a-3p, hsa-miR-34a-5p, hsa-miR-34b-3p, hsa-miR-34b-5p, hsa-miR-34c-3p, hsa-miR-34c-5p, hsa-miR-3591-3p, hsa-miR-361-3p, hsa-miR-361-5p, hsa-miR-3613-5p, hsa-miR-3615, hsa-miR-3619, hsa-miR-362-3p, hsa-miR-362-5p, hsa-miR-363-3p, hsa-miR-363-5p, hsa-mir-365a-3p, hsa-mir-365a-5p, hsa-mir-3653, hsa-miR-3656, hsa-miR-367-3p, hsa-miR-367-5p, hsa-miR-3676-3p, hsa-miR-369-3p, hsa-miR-369-5p, hsa-miR-370, hsa-miR-372, hsa-miR-373-3p, hsa-miR-373-5p, hsa-miR-374a-3p, hsa-miR-374a-5p, hsa-miR-374b-3p, hsa-miR-374b-5p, hsa-miR-375, hsa-miR-376a-2-5p, hsa-miR-376a-3p, hsa-miR-376a-5p, hsa-miR-376b-3p, hsa-miR-376c-3p, hsa-miR-377-3p, hsa-miR-377-5p, hsa-miR-378a-3p, hsa-miR-378a-5p, hsa-miR-378c, hsa-miR-378d, hsa-miR-379-3p, hsa-miR-379-5p, hsa-miR-30-3p, hsa-miR-380-5p, hsa-miR-381-3p, hsa-miR-382-3p, hsa-miR-382-5p, hsa-miR-383, hsa-miR-384, hsa-miR-3912, hsa-miR-3928, hsa-miR-409-3p, hsa-miR-409-5p, hsa-miR-410, hsa-miR-411-3p, hsa-miR-411-5p, hsa-miR-412, hsa-miR-421, hsa-miR-422a, hsa-miR-423-3p, hsa-miR-423-5p, hsa-miR-424-3p, hsa-miR-424-5p, hsa-miR-425-3p, hsa-miR-425-5p, hsa-miR-429, hsa-miR-4291, hsa-miR-431-5p, hsa-miR-432-5p, hsa-miR-433, hsa-miR-4421, hsa-miR-449a, hsa-miR-450a-5p, hsa-miR-450b-3p, hsa-miR-450b-5p, hsa-miR-4505, hsa-miR-4510, hsa-miR-4516, hsa-miR-451a, hsa-miR-452-3p, hsa-miR-452-5p, hsa-miR-4533, hsa-miR-4539, hsa-miR-454-3p, hsa-miR-454-5p, hsa-miR-455-3p, hsa-miR-455-5p, hsa-miR-4634, hsa-miR-4732-3p, hsa-miR-4732-5p, hsa-miR-4747-5p, hsa-miR-4792, hsa-miR-483-3p, hsa-miR-483-5p, hsa-miR-484, hsa-miR-485-5p, hsa-miR-486-3p, hsa-miR-486-5p, hsa-miR-489, hsa-miR-490, hsa-miR-491-3p, hsa-miR-491-5p, hsa-miR-492, hsa-miR-493-3p, hsa-miR-493-5p, hsa-miR-494, hsa-miR-495-3p, hsa-miR-496, hsa-miR-497-3p, hsa-miR-497-5p, hsa-miR-498, hsa-miR-499a-5p, hsa-miR-500a-3p, hsa-miR-501-3p, hsa-miR-501-5p, hsa-miR-502-3p, hsa-miR-502-5p, hsa-miR-503-5p, hsa-miR-504, hsa-miR-505-3p, hsa-miR-505-5p, hsa-miR-506-3p, hsa-miR-508-3p, hsa-miR-508-5p, hsa-miR-509-3p, hsa-miR-511, hsa-miR-512-5p, hsa-miR-513a-3p, hsa-miR-513a-5p, hsa-miR-513b, hsa-miR-514a-3p, hsa-miR-514a-5p, hsa-miR-515-5p, hsa-miR-516b-3p, hsa-miR-516b-5p, hsa-miR-517a-3p, hsa-miR-518a-3p, hsa-miR-518b, hsa-miR-518e-3p, hsa-miR-518e-5p, hsa-miR-518f-3p, hsa-miR-519a-5p, hsa-miR-519b-5p, hsa-miR-519c-3p, hsa-miR-519c-5p, hsa-miR-519d, hsa-miR-519e-5p, hsa-miR-520c-3p, hsa-miR-520e, hsa-miR-520f, hsa-miR-520g, hsa-miR-520h, hsa-miR-521, hsa-miR-522-5p, hsa-miR-523-5p, hsa-miR-525-3p, hsa-miR-532-3p, hsa-miR-532-5p, hsa-miR-539-5p, hsa-miR-541, hsa-miR-542-3p, hsa-miR-542-5p, hsa-miR-543, hsa-miR-545-3p, hsa-miR-545-5p, hsa-miR-548a-3p, hsa-miR-548d-3p, hsa-miR-548e, hsa-miR-548i, hsa-miR-548m, hsa-miR-549, hsa-miR-550a-3p, hsa-miR-550a-5p, hsa-miR-551b-3p, hsa-miR-551b-5p, hsa-miR-552, hsa-miR-553, hsa-miR-554, hsa-miR-557, hsa-miR-563, hsa-miR-564, hsa-miR-567, hsa-miR-569, hsa-miR-570-3p, hsa-miR-571, hsa-miR-572, hsa-miR-574-3p, hsa-miR-574-5p, hsa-miR-575, hsa-miR-576-3p, hsa-miR-576-5p, hsa-miR-577, hsa-miR-578, hsa-miR-580, hsa-miR-582-3p, hsa-miR-582-5p, hsa-miR-583, hsa-miR-584-5p, hsa-miR-585, hsa-miR-586, hsa-miR-589-3p, hsa-miR-589-5p, hsa-miR-590-3p, hsa-miR-590-5p, hsa-miR-593-3p, hsa-miR-593-5p, hsa-miR-595, hsa-miR-598, hsa-miR-601, hsa-miR-602, hsa-miR-603, hsa-miR-606, hsa-miR-608, hsa-miR-609, hsa-miR-611, hsa-miR-612, hsa-miR-613, hsa-miR-615-3p, hsa-miR-615-5p, hsa-miR-616-5p, hsa-miR-618, hsa-miR-619, hsa-miR-620, hsa-miR-623, hsa-miR-625-5p, hsa-miR-626, hsa-miR-627, hsa-miR-628-3p, hsa-miR-628-5p, hsa-miR-629-3p, hsa-miR-629-5p, hsa-miR-630, hsa-miR-631, hsa-miR-634, hsa-miR-635, hsa-miR-636, hsa-miR-638, hsa-miR-639, hsa-miR-641, hsa-miR-642a-3p, hsa-miR-642a-5p, hsa-miR-643, hsa-miR-645, hsa-miR-646, hsa-miR-647, hsa-miR-649, hsa-miR-650, hsa-miR-651, hsa-miR-652-3p, hsa-miR-653, hsa-miR-654-3p, hsa-miR-655, hsa-miR-656, hsa-miR-657, hsa-miR-658, hsa-miR-659-3p, hsa-miR-660-5p, hsa-miR-663a, hsa-miR-663b, hsa-miR-664a-3p, hsa-miR-664a-5p, hsa-miR-668, hsa-miR-671-5p, hsa-miR-675-3p, hsa-miR-675-5p, hsa-miR-7-1-3p, hsa-miR-7-2-3p, hsa-miR-7-5p, hsa-miR-708-3p, hsa-miR-708-5p, hsa-miR-718, hsa-miR-744-3p, hsa-miR-744-5p, hsa-miR-761, hsa-miR-765, hsa-miR-766, hsa-miR-767-3p, hsa-miR-769-3p, hsa-miR-769-5p, hsa-miR-770-5p, hsa-miR-874, hsa-miR-875, hsa-miR-876, hsa-miR-877-3p, hsa-miR-877-5p, hsa-miR-885-3p, hsa-miR-887, hsa-miR-888-3p, hsa-miR-889, hsa-miR-890, hsa-miR-891a, hsa-miR-891b, hsa-miR-9-3p, hsa-miR-9-5p, hsa-miR-92a-3p, hsa-miR-92b-3p, hsa-miR-92b-5p, hsa-miR-921, hsa-miR-922, hsa-miR-93-3p, hsa-miR-93-5p, hsa-miR-935, hsa-miR-940, hsa-miR-941, hsa-miR-942, hsa-miR-95, hsa-miR-96-3p, hsa-miR-96-5p, hsa-miR-98-5p, hsa-miR-99a-3p, hsa-miR-99a-5p, hsa-miR-99b-3p, and/or hsa-miR-99b-5p, or an analog thereof. The sequences of these miRNAs are well known in the art and may be found, for example, on the world wide web at mirbase.org.

In some currently preferred embodiments, the miRNA is hsa-let-7d-3p, hsa-miR-101-5p, hsa-miR-106b-3p, hsa-miR-1179, hsa-miR-125a-5p, hsa-miR-141-3p, hsa-miR-148a-3p, hsa-miR-16-5p, hsa-miR-192-5p, hsa-miR-195-5p, hsa-miR-19b-3p, hsa-miR-20a-5p, hsa-miR-200b-3p, hsa-miR-217, hsa-miR-223-3p, hsa-miR-371a-3p, hsa-miR-487b, hsa-miR-515-3p, hsa-miR-605, hsa-miR-20b-5p, hsa-miR-27b-3p, hsa-miR-103a-3p, hsa-miR-22-3p, hsa-miR-34a-5p, hsa-miR-130b-3p, hsa-miR-132-3p, hsa-miR-181b-5p, hsa-miR-211-5p, hsa-miR-148b-3p, hsa-miR-17-5p, hsa-miR-182-5p, hsa-miR-27a-3p, hsa-miR-301a-3p, hsa-miR-204-5p, hsa-miR-143-3p, hsa-miR-1, hsa-miR-9-5p, hsa-miR-30a-5p, hsa-miR-138-5p, hsa-miR-382-5p, hsa-miR-106a-5p, hsa-miR-107, hsa-miR-135a-5p, hsa-miR-93-5p, hsa-miR-21-5p, hsa-miR-155-5p, hsa-miR-181a-5p, hsa-miR-519d, hsa-miR-96-5p, hsa-miR-212-3p, hsa-miR-29a-3p, hsa-miR-98-5p, hsa-let-7c, hsa-let-7d-5p, hsa-miR-183-5p, hsa-miR-19a-3p, hsa-miR-196a-5p, hsa-miR-30b-5p, hsa-miR-378a-3p, hsa-miR-302c-5p, hsa-miR-30e-5p, hsa-miR-130a-3p, hsa-let-7e-5p, hsa-miR-216a-5p, hsa-miR-450a-5p, hsa-miR-26b-5p, hsa-miR-181c-5p, hsa-miR-186-5p, hsa-miR-519c-3p, hsa-let-7b-5p, hsa-miR-10b-5p, hsa-miR-125b-5p, hsa-miR-134, hsa-miR-137, hsa-miR-150-5p, hsa-miR-153, hsa-miR-15b-5p, hsa-miR-196b-5p, hsa-miR-23a-3p, hsa-miR-29c-3p, hsa-miR-373-3p, hsa-miR-7-5p, hsa-miR-214-3p, hsa-miR-421, hsa-miR-15a-5p, hsa-miR-193b-3p, hsa-miR-194-5p, hsa-miR-30d-5p, hsa-miR-424-5p, hsa-miR-454-3p, hsa-miR-545-3p, hsa-miR-485-5p, hsa-miR-335-5p, hsa-miR-133a, hsa-miR-222-3p, hsa-miR-494, hsa-miR-498, hsa-miR-513a-5p, hsa-miR-92a-3p, hsa-miR-495-3p, hsa-miR-503-5p, hsa-miR-539-5p, hsa-miR-16-2-3p, hsa-miR-302b-5p, hsa-miR-425-3p, hsa-miR-99a-3p, hsa-let-7a-3p, hsa-miR-126-3p, hsa-miR-20a-3p, hsa-miR-499a-5p, hsa-let-7g-5p, hsa-miR-152, hsa-miR-26a-5p, hsa-miR-124-3p, hsa-miR-203a, hsa-miR-24-3p, hsa-miR-301b, hsa-miR-590-3p, hsa-miR-325, hsa-miR-552, hsa-miR-185-5p, hsa-miR-455-3p, hsa-miR-583, hsa-miR-122-5p, hsa-miR-1305, hsa-miR-139-5p, hsa-miR-146a-5p, hsa-miR-18a-5p, hsa-miR-18b-5p, hsa-miR-199b-5p, hsa-miR-340-5p, hsa-miR-34c-5p, hsa-miR-423-3p, hsa-miR-489, hsa-miR-520f, hsa-miR-520g, hsa-miR-668, hsa-let-7a-5p, hsa-let-7f-5p, hsa-miR-110a-3p, hsa-miR-135b-5p, hsa-miR-144-3p, hsa-miR-181d, hsa-miR-200c-3p, hsa-miR-218-5p, hsa-miR-23b-3p, hsa-miR-25-3p, hsa-miR-29b-3p, hsa-miR-383, hsa-miR-202-3p, hsa-miR-381-3p, hsa-miR-377-3p, hsa-miR-452-5p, hsa-miR-501-3p, hsa-miR-514a-3p, hsa-miR-654-3p, hsa-let-7b-3p, hsa-miR-125a-3p, hsa-miR-133b, hsa-miR-199a-3p, hsa-miR-30c-5p, hsa-miR-335-3p, hsa-miR-374a-5p, hsa-miR-410, hsa-miR-429, hsa-miR-497-5p, hsa-miR-513a-3p, hsa-miR-542-3p, hsa-miR-653, hsa-miR-122-3p, hsa-miR-1178-3p, hsa-miR-191-5p, hsa-miR-214-5p, hsa-miR-302d-5p, hsa-miR-572, hsa-miR-574-3p, hsa-miR-26a-2-3p, hsa-miR-611, hsa-let-7f-1-3p, hsa-let-7i-3p, hsa-miR-100-5p, hsa-miR-106b-5p, hsa-miR-132-5p, hsa-miR-135b-3p, hsa-miR-136-3p, hsa-miR-150-3p, hsa-miR-154-3p, hsa-miR-15a-3p, hsa-miR-15b-3p, hsa-miR-16-1-3p, hsa-miR-181a-2-3p, hsa-miR-181c-3p, hsa-miR-186-3p, hsa-miR-195-3p, hsa-miR-20b-3p, hsa-miR-223-5p, hsa-miR-224-3p, hsa-miR-24-1-5p, hsa-miR-24-2-5p, hsa-miR-27a-5p, hsa-miR-27b-5p, hsa-miR-29b-1-5p, hsa-miR-302a-5p, hsa-miR-3065-5p, hsa-miR-30d-3p, hsa-miR-34a-3p, hsa-miR-373-5p, hsa-miR-374a-3p, hsa-miR-376a-5p, hsa-miR-378a-5p, hsa-miR-424-3p, hsa-miR-451a, hsa-miR-452-3p, hsa-miR-493-5p, hsa-miR-500a-3p, hsa-miR-502-3p, hsa-miR-516b-3p, hsa-miR-518e-3p, hsa-miR-518f-3p, hsa-miR-519a-5p, hsa-miR-519b-5p, hsa-miR-521, hsa-miR-523-5p, hsa-miR-545-5p, hsa-miR-585, hsa-miR-7-2-3p, hsa-miR-93-3p, hsa-miR-96-3p, and/or hsa-miR-99b-3p, or an analog thereof.

In some currently even more preferred embodiments, the miRNA is hsa-let-7d-3p, hsa-miR-101-5p, hsa-miR-106b-3p, hsa-miR-1179, hsa-miR-125a-5p, hsa-miR-141-3p, hsa-miR-148a-3p, hsa-miR-16-5p, hsa-miR-192-5p, hsa-miR-195-5p, hsa-miR-19b-3p, hsa-miR-20a-5p, hsa-miR-200b-3p, hsa-miR-217, hsa-miR-223-3p, hsa-miR-371a-3p, hsa-miR-487b, hsa-miR-515-3p, and/or hsa-miR-605, or an analog thereof.

In certain aspects, the compositions of invention comprise miRNA modulator elements for modulating thermogenesis. As used herein, the term “modulate” refers to increasing or decreasing a parameter. For example, to modulate the activity of a protein that protein's activity could be increased or decreased. In exemplary embodiments, the miRNA analog modulates the activity of at least one mitochondrial uncoupler (e.g., UCP1, UCP2 and/or UCP3). Such methods and compositions are particularly useful for treating obesity. See U.S. application Ser. No. 13/826,775, filed on Mar. 14, 2013; and International Application Serial No. PCT/US2013/037579 filed on Apr. 22, 2013, each of which hereby incorporated by reference in their entirety. As used herein, the term “activity” of a mitochondrial uncoupler or thermogenic regulator refers to any measurable biological activity including, without limitation, mRNA expression, protein expression, or respiratory chain uncoupling.

Mitochondrial uncoupling proteins (UCP) are members of the family of mitochondrial anion carrier proteins (MACP). UCPs separate oxidative phosphorylation from ATP synthesis with energy dissipated as heat (also referred to as the “mitochondrial proton leak”). UCPs facilitate the transfer of anions from the inner to the outer mitochondrial membrane and the return transfer of protons from the outer to the inner mitochondrial membrane generating heat in the process. UCPs are the primary proteins responsible for thermogenesis and heat dissipation. Uncoupling Protein 1 (UCP1) (SEQ ID NO:1), also named thermogenin, is a BAT specific protein responsible for thermogenesis and heat dissipation. UCP2 (SEQ ID NO:2) is another Uncoupling Protein also expressed in adipocytes. UCPs are part of network of thermogenic regulator proteins.

Modulation of thermogenic regulators to induce BAT differentiation and/or mitochondrial uncoupling provides a method to induce thermogenesis in a subject and, hence, to treat obesity. However, chemical pharmacologic approaches cannot target these molecules, as they do not belong to the classic ‘target classes’ (kinases, ion channels, G-protein coupled receptors, etc.) that dominate the ‘druggable space’ of traditional drug discovery. Accordingly, the invention provides novel methods and compositions for modulating these thermogenic regulators using miRNA agents.

In certain embodiments, miRNA modulators are employed to upregulate the activity of a mitochondrial uncoupler (e.g., the mRNA expression level, protein expression level, or mitochondrial uncoupling activity). Upregulation of a mitochondrial uncoupler can be achieved in several ways. In one embodiment, the miRNA analog directly inhibits the activity of a naturally occurring miRNA that is responsible for downregulation of the activity (e.g., the mRNA expression level, protein expression level) of the mitochondrial uncoupler. In certain embodiments, the miRNA agent directly binds to the mRNA or promoter region of the mitochondrial uncoupler. For example, the miRNA agent may directly bind to the 5′UTR or coding sequence of the mRNA of at least one mitochondrial uncoupler.

In certain embodiments, the miRNA agent modulates the activity of an activator or repressor of a mitochondrial uncoupling protein. In another embodiment, the miRNA analog upregulates the activity (e.g., the mRNA expression level, protein expression level) of an activator of the mitochondrial uncoupler. This upregulation can be achieved, for example, by directly inhibiting the activity of a naturally occurring miRNA that is responsible for downregulation of the expression of the activator. In other embodiment, the miRNA analog downregulates the activity (e.g., the mRNA expression level, protein expression level) of a repressor of the mitochondrial uncoupler. This downregulation can be achieved, for example, by directly inhibiting the expression of a repressor of a mitochondrial uncoupler using a miRNA analog.

In certain embodiments, the invention employs miRNA analogs for the modulation of thermogenic regulators (e.g., mitochondrial uncouplers, such as UCP1 and UCP2). miRNA analogs, suitable for use in the methods disclosed herein, included, without limitation, miRNA, agomirs, antagomirs, miR-masks, miRNA-sponges, siRNA (single- or double-stranded), shRNA, antisense oligonucleotides, ribozymes, or other oligonucleotide mimetics which hybridize to at least a portion of a target nucleic acid and modulate its function. As used herein, the term “thermogenic regulator” refers to a protein (or the encoding nucleic acid) that regulates thermogenesis either directly or indirectly. The term encompasses mitochondrial uncouplers, and also activators and repressors of mitochondrial uncouplers.

In certain embodiments, the miRNA analogs are miRNA molecules or synthetic derivatives thereof (e.g., agomirs and antagomirs). In one particular embodiment, the miRNA analog is a miRNA. miRNAs are a class of small (e.g., 18-24 nucleotides) non-coding RNAs that exist in a variety of organisms, including mammals, and are conserved in evolution. miRNAs are processed from hairpin precursors of about 70 nucleotides which are derived from primary transcripts through sequential cleavage by the RNAse III enzymes drosha and dicer. Many miRNAs can be encoded in intergenic regions, hosted within introns of pre-mRNAs or within ncRNA genes. Many miRNAs also tend to be clustered and transcribed as polycistrons and often have similar spatial temporal expression patterns. In general, miRNAs are post-transcriptional regulators that bind to complementary sequences on a target gene (mRNA or DNA), resulting in gene silencing by, e.g., translational repression or target degradation. One miRNA can target many different genes simultaneously.

Exemplary miRNA molecules for use in the disclosed methods include without limitation those disclosed herein. Additional miRNAs that modulate regulator molecules may be identified using publicly available Internet tools that predict miRNA targets. Modulation of a single miRNA can promote the formation of adipocytes from adipogenic precursor cells. Pathway-specific miRNAs that target multiple genes within one discrete signaling pathway are preferred, rather than universal miRNAs that are involved in many signaling pathways, functions or processes.

The term “agomir” refers to a synthetic oligonucleotide or oligonucleotide mimetic that functionally mimics a miRNA. An agomir can be an oligonucleotide with the same or similar nucleic acid sequence to a miRNA or a portion of a miRNA. In certain embodiments, the agomir has 1, 2, 3, 4, 5, 6, 7, 8, 9 or 10 nucleotide differences from the miRNA that it mimics. Further, agomirs can have the same length, a longer length or a shorter length than the miRNA that it mimics. In certain embodiments, the agomir has the same sequence as 6-8 nucleotides at the 5′ end of the miRNA it mimics. In other embodiments, an agomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length or any range derivable therein. In other embodiments, an agomir can be 5-10, 6-8, 10-20, 10-15 or 5-500 nucleotides in length or any range derivable therein. In certain embodiments, agomirs include any of the sequences of miRNAs disclosed herein. These chemically modified synthetic RNA duplexes include a guide strand that is identical or substantially identical to the miRNA of interest to allow efficient loading into the miRISC complex, whereas the passenger strand is chemically modified to prevent its loading to the Argonaute protein in the miRISC complex (Thorsen, et al., 2012; Broderick, et al., 2011).

The term “antagomir” refers to a synthetic oligonucleotide or oligonucleotide mimetic having complementarity to a specific microRNA, and which inhibits the activity of that miRNA. The term “antimir” is synonymous with the term “antagomir”. In certain embodiments, the antagomir has 1, 2, 3, 4, 5, 6, 7, 8, 9 or 10 nucleotide differences from the miRNA that it inhibits. Further, antagomirs can have the same length, a longer length or a shorter length than the miRNA that it inhibits. In certain embodiments, the antagomir hybridizes to 6-8 nucleotides at the 5′ end of the miRNA it inhibits. In other embodiments, an antagomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length or any range derivable therein. In other embodiments, an antagomir can be 5-10, 6-8, 10-20, 10-15 or 5-500 nucleotides in length or any range derivable therein. In certain embodiments, antagomirs include nucleotides that are complementary to any of the sequences of miRNAs disclosed herein. The antagomirs are synthetic reverse complements that tightly bind to and inactivate a specific miRNA. Various chemical modifications are used to improve nuclease resistance and binding affinity. The most commonly used modifications to increase potency include various 2′sugar modifications, such as 2′-O-Me, 2′-O-methoxyethyl (2′-MOE), or 2′-fluoro(2′-F). The nucleic acid structure of the miRNA can also be modified into a locked nucleic acid (LNA) with a methylene bridge between the 2′oxygen and the 4′ carbon to lock the ribose in the 3′-endo (North) conformation in the A-type conformation of nucleic acids (Lennox, et al., 2011; Bader, et al. 2011). This modification significantly increases both target specificity and hybridization properties of the molecules.

In certain embodiments, the miRNA analogs are oligonucleotide or oligonucleotide mimetics that inhibit the activity of one or more miRNA. Examples of such molecules include, without limitation, antagomirs, interfering RNA, antisense oligonucleotides, ribozymes, miRNA sponges and miR-masks. As used herein, the term “antisense oligonucleotide” refers to a synthetic oligonucleotide or oligonucleotide mimetic that is complementary to a DNA or mRNA sequence (e.g., an miRNA). In one particular embodiment, the miRNA analog is an antagomir. In general, antagomirs are chemically modified antisense oligonucleotides that bind to a target miRNA and inhibit miRNA function by prevent binding of the miRNA to its cognate gene target. Antagomirs can include any base modification known in the art. In one particular embodiment, the antagomir inhibits the activity of human miR-22 (van Rooij, et al., 2012; Snead, et al., 2012; Czech, et al., 2011).

In certain embodiments, the miRNA analogs are 10 to 50 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range derivable there within.

In certain embodiments, the miRNA analogs are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

In certain embodiments, the miRNA analogs comprise at least one nucleotide modified at the 2′ position of the sugar, most preferably a 2′-0-alkyl, 2′-0-alkyl-0-alkyl or 2′-fluoro-modified nucleotide. In other preferred embodiments, RNA modifications include 2′-fluoro, 2′-amino and 2′ O-methyl modifications on the ribose of pyrimidines, a basic residue or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make an oligonucleotide more resistant to nuclease digestion, thereby prolonging in vivo half-life. Specific examples of modified oligonucleotides include those comprising backbones comprising, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜0˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH); amide backbones (De Mesmaeker et al., 1995); morpholino backbone structures (Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone (Nielsen, et al., 1991), each of which is herein incorporated by reference in its entirety. Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321, 131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050, each of which is herein incorporated by reference in its entirety. Morpholino-based oligomeric compounds are known in the art described in Braasch & Corey, 2002; Genesis, 2001; Heasman, 2002; Nasevicius, et al., 2000; Lacerra, et al., 2000 and U.S. Pat. No. 5,034,506, issued Jul. 23, 1991, each of which is herein incorporated by reference in its entirety. Cyclohexenyl nucleic acid oligonucleotide mimetics are described in Wang et al., 2000, the contents of which is incorporated herein in its entirety.

Modified oligonucleotide backbones that do not include a phosphorus atom therein have backbones that are formed by short chain alkyl or cycloalkyl intemucleoside linkages, mixed heteroatom and alkyl or cycloalkyl intemucleoside linkages, or one or more short chain heteroatomic or heterocyclic intemucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In certain embodiments, miRNA analogs comprise one or more substituted sugar moieties, e.g., one of the following at the 2′ position: OH, SH, SCH₃, F, OCN, OCH₃ OCH₃, OCH₃ 0 (CH₂)n CH₃, 0(CH₂)n NH₂ or 0(CH₂)n CH₃ where n is from 1 to about 10; Ci to CIO lower alkyl, alkoxyalkoxy, substituted lower alkyl, alkaryl or aralkyl; CI; Br; CN; CF3; OCF3; 0-, S-, or N-alkyl; 0-, S-, or N-alkenyl; SOCH3; S02 CH3; ON02; N02; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacokinetic/pharmacodynamic properties of an oligonucleotide and other substituents having similar properties. A preferred modification includes 2′-methoxyethoxy [2′-0-CH₂CH₂OCH₃, also known as 2′-0-(2-methoxyethyl)]. Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

In certain embodiments, miRNA analogs comprise one or more base modifications and/or substitutions. As used herein, “unmodified” or “natural” bases include adenine (A), guanine (G), thymine (T), cytosine (C) and uracil (U). Modified bases include, without limitation, bases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic bases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine (Kornberg, 1980; Gebeyehu, et al., 1987). A “universal” base known in the art, e.g., inosine, can also be included. 5-Me-C substitutions can also be included. These have been shown to increase nucleic acid duplex stability by 0.6-1.2OC (Sanghvi, et al., 1993). Further suitable modified bases are described in U.S. Pat. No. 3,687,808, as well as U.S. Pat. Nos. 4,845,205; 5,130,302; 5,134,066; 5,175, 273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

It is not necessary for all positions in a given oligonucleotide to be uniformly modified, and in fact more than one of the aforementioned modifications may be incorporated in a single oligonucleotide or even at within a single nucleoside within an oligonucleotide.

In certain embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al., Science, 1991.

Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al., 1991.

In certain embodiments, the miRNA agent is linked (covalently or non-covalently) to one or more moieties or conjugates that enhance the activity, cellular distribution, or cellular uptake of the oligonucleotide. Such moieties include, without limitation, lipid moieties such as a cholesterol moiety (Letsinger et al., 1989), cholic acid (Manoharan et al., 1994), a thioether, e.g., hexyl-S-tritylthiol (Manoharan et al., 1992; Manoharan et al, 1993), a thiocholesterol (Oberhauser et al., 1992), an aliphatic chain, e.g., dodecandiol or undecyl residues (Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995a; Shea et al., 1990), a polyamine or a polyethylene glycol chain (Mancharan et al., 1995b), or adamantane acetic acid (Manoharan et al., 1995a), a palmityl moiety (Mishra et al., 1995), or an octadecylamine or hexylamino-carbonyl-t oxycholesterol moiety (Crooke et al., 1996), each of which is herein incorporated by reference in its entirety. See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety.

The miRNA analogs must be sufficiently complementary to the target mRNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of a miRNA analog is capable of hydrogen bonding with a base at the corresponding position of a target nucleic acid sequence, then the bases are considered to be complementary to each other at that position. In certain embodiments, 100% complementarity is not required. In other embodiments, 100% complementarity is required.

miRNA analogs for use in the methods disclosed herein can be designed using routine methods. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting a gene. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the miRNA agent contains about 5 to about 30 nucleotides). In some embodiments, target segments are represented by RNA sequences that comprise at least the 5 consecutive nucleotides from the 3′-terminus of one of the seed sequence (the remaining nucleotides being a consecutive stretch of the same miRNA beginning immediately downstream of the 3′-terminus of the target segment and continuing until the miRNA agent contains about 5 to about 30 nucleotides). As used herein, the term “seed sequence” refers to a 6-8 nucleotide (nt) long substring within the first 8 nt at the 5-end of the miRNA (i.e., seed sequence) that is an important determinant of target specificity.

One having skill in the art armed with the sequences provided in U.S. Application Ser. No. 61/636,059, filed on Apr. 20, 2012, will be able, without undue experimentation, to identify further preferred regions to target using miRNA analogs. Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target nucleic acid sequences), to give the desired effect.

In certain embodiments, miRNA agents used to practice this invention are expressed from a recombinant vector. Suitable recombinant vectors include, without limitation, DNA plasmids, viral vectors or DNA minicircles. Generation of the vector construct can be accomplished using any suitable genetic engineering techniques well known in the art. In certain embodiments, miRNA agents used to practice this invention are synthesized in vitro using chemical synthesis techniques.

### C. ADIPOSE-SPECIFIC TARGETING

In some embodiments, the present invention provides compositions and methods for targeted delivery of miRNA modulators to adipose tissue, e.g., white adipose tissue (WAT). Specifically, the goal is to selectively deliver miRNA analogs to adipose tissue. Human subcutaneous adipose tissue contains several cell types, any of which may be selectively targeted with the compositions of the invention. For example, in certain embodiments, the target cell is an adipocyte. In other embodiments, the target cell may be an adipocyte precursor such as a pre-adipocyte or adipose tissue mesenchymal stem cell (ATMSC). ATMSCs possess the ability to differentiate into multiple lineages, such as adipocytes, osteocytes, and chondrocytes and are present in human subcutaneous adipose tissue in appreciable quantities. Human ATMSCs can be reprogrammed to become brown adipocytes (BAT) via modulation of a defined set of transcription factors.

In certain embodiments, the compositions of the invention bind to an adipose-target cell comprising one or more ATMSC-positive surface markers. Exemplary ATMSC-positive surface markers include CD9 (tetraspan), CD10 (MME), CD13 (ANPEP), CD29 ((3-1 integrin), CD36 (FAT), CD44 (hyaluronate), CD49d (α-4 integrin), CD54 (ICAM-1), CD55 (DAF), CD59, CD73 (NT5E), CD90 (Thy1), CD91 (LPR1), CD105 (SH2, Endoglin), CD137, CD146 (Muc 18), CD166 (ALCAM), and HLA-ABC.

In other embodiments, the compositions of the invention selectively bind to subcutaneous or white adipose tissue (WAT). By selectively binding to WAT, compositions of the invention can facilitate targeted delivery of thermogenic miRNA modulators which promote conversion of white adipocyte to thermogenic brite or brown or beige adipocytes (BAT). Exemplary WAT-positive markers include adiponectin, caveolin-1, caveolin-2, CD36 (FAT), CLH-22 (clathrin heavy chain chr. 22), FABP4 (adipocyte protein 2, ap2), SLC27A1 (FATP1), SLC27A2 (FATP2), GLUT4 (glucose transporter 4), perilipin 1, perilipin 2, and resistin.

In yet other embodiments, the compositions of the invention bind to an adipose target cell comprising cellular markers (including several lipid transporters) that are preferentially expressed at the surface of adipocytes. Exemplary adipocyte cellular markers include caveolin-1 (CAV1), caveolin-2 (CAV2), CD10 (MME), CD36 (FAT), CD90 (Thy-1), CD91 (low density lipoprotein receptor-related protein 1, LRP1), CD140A (platelet-derived growth factor receptor, alpha polypeptide, PDGFRA), CD140B (platelet-derived growth factor receptor, alpha polypeptide, PDGFRB), CD146 (cell surface glycoprotein MUC18, MCAM), CD166 (activated leukocyte cell adhesion molecule, ALCAM), CLH-22 (clathrin heavy chain chromosome 22), DCN (decorin), DPT (dermatopontin), FABP4 (fatty acid binding protein 4), GLUT4 (glucose transporter 4, SLC2A4), LAMP1 (lysosomal-associated membrane protein 1), LAMP2 (lysosomal-associated membrane protein 2), NPR1 (Natriuretic peptide receptor A), SLC27A1 (FATP1), and SLC27A2 (FATP2). See Table 1. Other specific (positive) markers of adipose tissue include adiponectin, BMP7, BMP8b, CIDEC, FGF 17, FGF 19, INSG1 (Insulin-induced gene 1), leptin, LPL, MetAP2, NR1H3 (LXRA), perilipin 1, perilipin 2, perilipin 3, PPARG, RBP4, and resistin.

TABLE 1
Human adipocyte surface markers

| | | | Mean tissue | Adipocyte | Expression |
|---|---|---|---|---|---|
| | Entrez | | mRNA | mRNA | Ratio |
| | Gene | | expression | expression | Adipocyte/ |
| Name | ID | Ensembl Gene ID | level | level | Mean |
| DPT (Dermatopontin) | 1805 | ENSG00000143196 | 20 | 747 | 37.4 |
| CD10 (MME) | 4311 | ENSG00000196549 | 20 | 707 | 35.4 |
| FABP4 | 2167 | ENSG00000170323 | 161 | 5407 | 33.6 |
| CD140B (PDGFRB) | 5159 | ENSG00000113721 | 22 | 274 | 12.5 |
| CD36 (FAT) | 948 | ENSG00000135218 | 71 | 885 | 12.5 |
| Caveolin 1 (CAV1) | 857 | ENSG00000105974 | 70 | 827 | 11.8 |
| Decorin (DCN) | 1634 | ENSG00000011465 | 566 | 6342 | 11.2 |
| CD140A (PDGFRA) | 5156 | ENSG00000134853 | 101 | 923 | 9.1 |
| CD91 (LRP1) | 4035 | ENSG00000123384 | 100 | 718 | 7.2 |
| Caveolin 2 (CAV2) | 858 | ENSG00000105971 | 46 | 259 | 5.6 |
| CD90 (THY1) | 7070 | ENSG00000154096 | 60 | 299 | 5.0 |
| CD146 (MCAM) | 4162 | ENSG00000076706 | 8 | 31 | 3.9 |
| CD166 (ALCAM) | 214 | ENSG00000170017 | 27 | 87 | 3.2 |
| LAMP1 | 3916 | ENSG00000185896 | 25 | 79 | 3.2 |
| NPR1 | 4881 | ENSG00000169418 | 133 | 189 | 1.4 |
| CLH-22 | 1213 | ENSG00000141367 | 1085 | 1040 | 1.0 |
| GLUT4 (SLC2A4) | 6517 | ENSG00000181856 | 7 | 7 | 1.0 |
| SLC27A1 (FATP1) | 376497 | ENSG00000130304 | 5 | 5 | 1.0 |
| SLC27A2 (FATP2) | 11001 | ENSG00000140284 | 18 | 11 | 0.6 |
| LAMP2 | 3920 | ENSG00000005893 | 37 | 16 | 0.4 |

In certain embodiments, compositions of the invention may comprise targeting elements which selectively bind one or more the above-identified markers, thus enhancing the selective delivery of miRNA modulators to adipocytes in order to enhance thermogenesis. Knowledge of the cell surface markers allows for their isolation by Flow Cytometry Cell Sorting (FACS) for subsequent screening and selection of targeting aptamers, for example by the SELEX or Cell-SELEX processes.

In some embodiments, aptamers are used to achieve this cell-specific delivery. An aptamer is an isolated or purified nucleic acid that binds with high specificity and affinity to a target through interactions other than Watson-Crick base pairing. An aptamer has a three dimensional structure that provides chemical contacts to specifically bind to a target. Unlike traditional nucleic acid binding, aptamer binding is not dependent upon a conserved linear base sequence, but rather a unique secondary or tertiary structure. That is, the nucleic acid sequences of aptamers are non-coding sequences. Any coding potential that an aptamer may possess is entirely fortuitous and plays no role whatsoever in the binding of an aptamer to a target. A typical minimized aptamer is 5-15 kDa in size (15-45 nucleotides), binds to a target with nanomolar to sub-nanomolar affinity, and discriminates against closely related targets (e.g., aptamers will typically not bind to other proteins from the same gene or functional family).

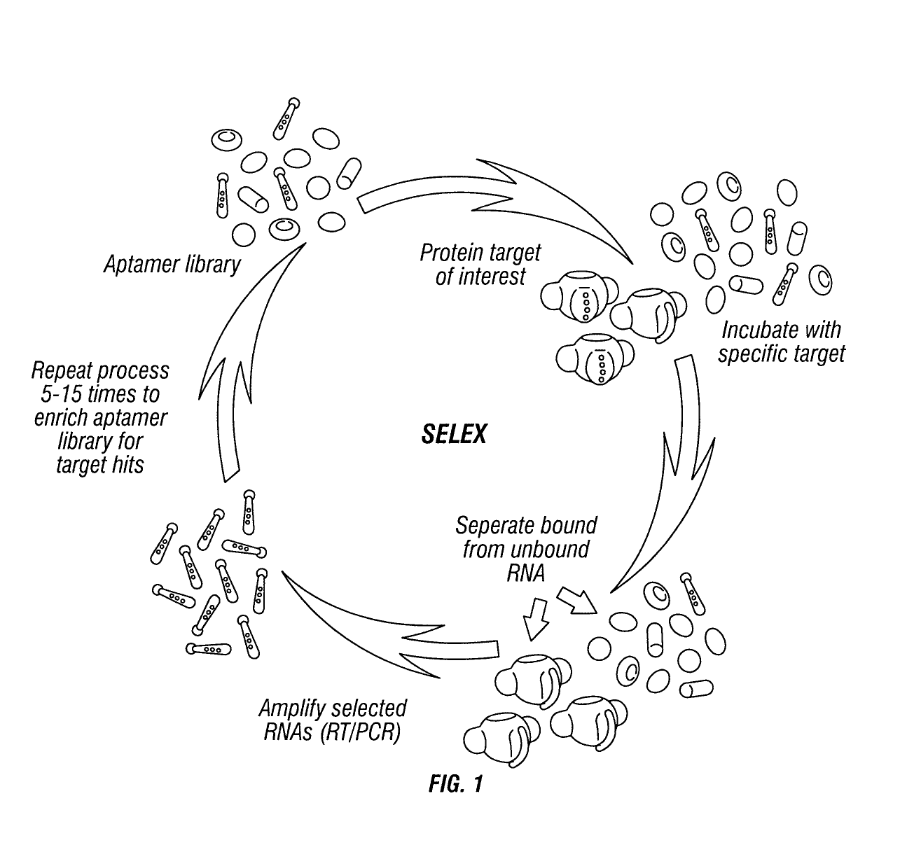

In exemplary embodiments, compositions of the invention comprise an aptamer targeting element that selectively binds to at least one of the positive markers identified above. Preferably, the aptamer element does not bind to any of the negative markers identified above. Such aptamers may be identified by any means known in the art, e.g., the SELEX™ process. Systematic Evolution of Ligands by EXponential Enrichment, or SELEX™ (FIG. 1), is a method for the in vitro evolution of nucleic acid molecules with highly specific binding to target molecules, see U.S. Pat. Nos. 5,270,163 and 5,475,096, each of which is incorporated herein by reference in its entirety. More specifically, starting with a mixture containing a starting pool of nucleic acids, the SELEX™ method includes the steps of: (a) contacting the mixture with a target under conditions favorable for binding; (b) partitioning unbound nucleic acids from those nucleic acids that have bound to the target; (c) amplifying the bound nucleic acids to yield a ligand-enriched mixture of nucleic acids; and (d) reiterating the steps of contacting, partitioning, and amplifying through as many cycles as desired to yield highly specific, high affinity aptamers to the target. Additional rounds of selection progressively favor the best ligands until the resulting nucleic acid mixture is predominantly composed of only one or a few sequences. By performing iterative cycles of selection and amplification, the SELEX™ process may be used to obtain aptamers, also referred to in the art as “nucleic acid ligands”, with any desired level of target binding affinity.

In those instances where transcribed aptamers, such as DNA or RNA aptamers, are being selected, the amplification step of the SELEX™ method includes the steps of: (i) reverse transcribing the nucleic acids dissociated from the nucleic acid-target complexes or otherwise transmitting the sequence information into a corresponding DNA sequence; (ii) PCR amplification; and (iii) transcribing the PCR amplified nucleic acids or otherwise transmitting the sequence information into a corresponding RNA sequence before restarting the process.

There are numerous modifications that may be made to an aptamer either before, during, and/or after the SELEX™ process, which are known in the art. Pre-SELEX™ process modifications or those made by incorporation into the SELEX™ process yield aptamers with both specificity for their target and improved stability. Post-SELEX™ process modifications made to already identified aptamers may result in further improved stability. Pre-SELEX™ process modifications usually lead to global changes in the aptamer, while post-SELEX™ process modifications lead to local changes in the aptamer.

The starting pool of nucleic acids can be random or partially random or non-random, modified or unmodified DNA, RNA, or DNA/RNA hybrids, and acceptable modifications include modifications at a base, sugar, and/or internucleotide linkage. The oligonucleotides of the starting pool preferably include a randomized sequence portion as well as fixed sequences necessary for efficient amplification. Typically, the oligonucleotides of the starting pool contain fixed 5′ and 3′ terminal sequences that flank an internal region of 30-50 random nucleotides. The randomized nucleotides can be produced in a number of ways, including chemical synthesis, size selection from randomly cleaved cellular nucleic acids, mutagenesis, solid phase oligonucleotide synthesis techniques, or solution phase methods (such as trimester synthesis methods). The random portion of the oligonucleotide can be of any length and can comprise ribonucleotides and/or deoxynucleotides, and can include modified or non-natural nucleotides or nucleotide analogs. The composition of the starting pool is dependent upon the desired properties of the final aptamer. Selections can be performed with nucleic acid sequences incorporating modified nucleotides to, e.g., stabilize the aptamers against degradation in vivo. For example, resistance to nuclease degradation can be greatly increased by the incorporation of modifying groups at the 2′-position. The starting library of oligonucleotides may be generated by automated chemical synthesis on a DNA synthesizer.

The SELEX process can be modified to incorporate a wide variety of modified nucleotides in order to generate a chemically-modified aptamer. For example, the aptamer may be synthesized entirely of modified nucleotides or with a subset of modified nucleotides. The modifications can be the same or different. Some or all nucleotides may be modified, and those that are modified may contain the same modification. For example, all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or pools of transcripts, are generated using any combination of modifications, including for example, ribonucleotides (2′-OH), deoxyribonucleotides (2′-deoxy), 2′-amino nucleotides (2′-NH₂), 2′-fluoro nucleotides (2′-F) and 2′-O-methyl (2′-OMe) nucleotides.

A SELEX process can employ a transcription mixture containing modified nucleotides in order to generate a modified aptamer. For example, a transcription mixture may contain only 2′-OMe A, G, C and U and/or T triphosphates (2′-OMe ATP, 2′-OMe UTP and/or 2′-OMe TTP, 2′-OMe CTP and 2′-OMe GTP), referred to as an MNA or mRmY mixture. Aptamers selected therefrom are referred to as MNA aptamers or mRmY aptamers and contain only 2′-O-methyl nucleotides. A transcription mixture containing all 2′-OH nucleotides is referred to as a “rN” mixture, and aptamers selected therefrom are referred to as “rN”, “rRrY” or RNA aptamers. A transcription mixture containing all deoxy nucleotides is referred to as a “dN” mixture, and aptamers selected therefrom are referred to as “dN”, “dRdY” or DNA aptamers. Alternatively, a subset of nucleotides (e.g., C, U and/or T) may comprise a first modified nucleotides (e.g, 2′-OMe) nucleotides and the remainder (e.g., A and G) comprise a second modified nucleotide (e.g., 2′-OH or 2′-F). For example, a transcription mixture containing 2′-F U and 2′-OMe A, G and C is referred to as a “fUmV mixture, and aptamers selected therefrom are referred to as “fUmV” aptamers. A transcription mixture containing 2′-F A and G, and 2′-OMe C and U and/or T is referred to as a “fRmY” mixture, and aptamers selected therefrom are referred to as “fRmY” aptamers. A transcription mixture containing 2′-F A and 2′-OMe C, G and U and/or T is referred to as a “fAmB” mixture, and aptamers selected therefrom are referred to as “fAmB” aptamers.

In addition to the Pre-SELEX™ process modifications discussed above, one of skill in the art can improve already identified aptamers using post-SELEX™ process modifications. Examples of post-SELEX™ process modifications include, but are not limited to, truncation, deletion, substitution, or modification of a sugar or base or internucleotide linkage, capping, and PEGylation. In addition, the sequence requirements of an aptamer may be explored through doped reselections or aptamer medicinal chemistry. Doped reselections are carried out using a synthetic, degenerate pool that has been designed based on the aptamer of interest. The level of degeneracy usually varies from about 70-85% from the aptamer of interest. In general, sequences with neutral mutations are identified through the doped reselection process. Aptamer medicinal chemistry is an aptamer improvement technique in which sets of variant aptamers are chemically synthesized. These variants are then compared to each other and to the parent aptamer. Aptamer medicinal chemistry is used to explore the local, rather than global, introduction of substituents. For example, the following modifications may be introduced: modifications at a sugar, base, and/or internucleotide linkage, such as 2′-deoxy, 2′-ribo, or 2′-O-methyl purines or pyrimidines, phosphorothioate linkages may be introduced between nucleotides, a cap may be introduced at the 5′ or 3′ end of the aptamer (such as 3′ inverted dT cap) to block degradation by exonucleases, or a polyethylene glycol (PEG) element may be added to the aptamer to increase the half-life of the aptamer in the subject.

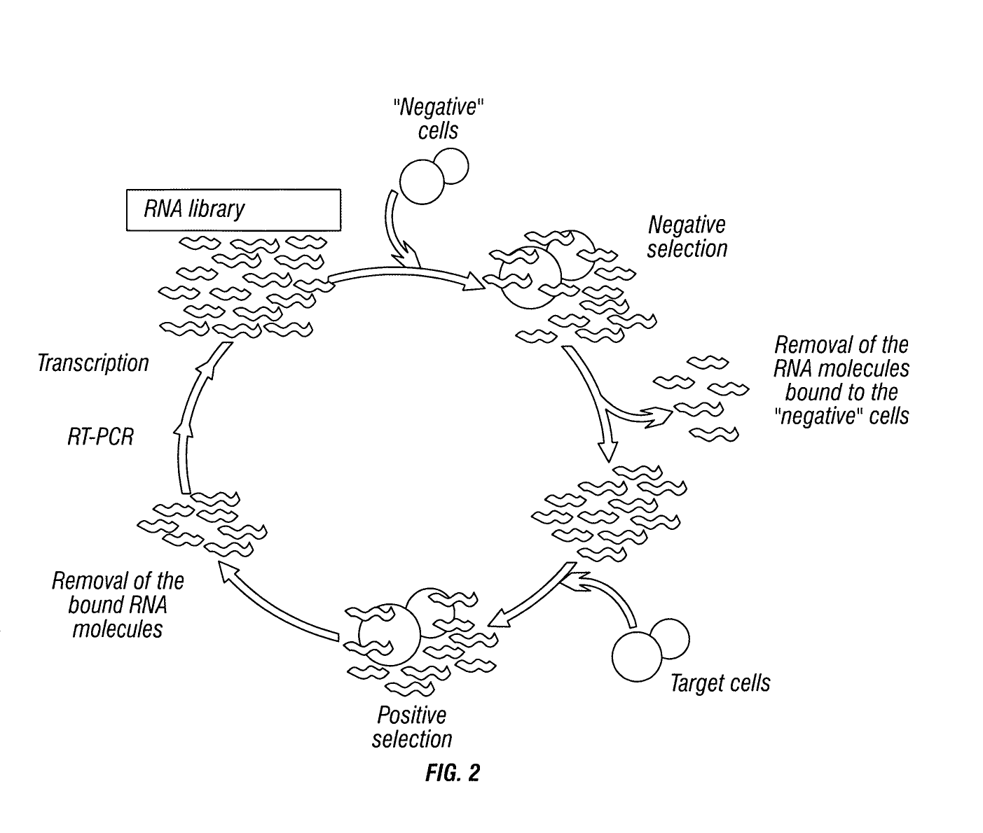

Variations of the SELEX process may also be used to identify aptamers. For example, one may use agonist SELEX, toggle SELEX, cell SELEX, 2′-Modified SELEX, or Counter SELEX. Each of these variations of the SELEX process is known in the art. The most preferred SELEX method used in the compositions and methods of the invention is Cell-SELEX, a variation of the SELEX™ process that is shown in FIG. 2. In general, SELEX uses a purified protein as its target. However, cell surface receptors are difficult to purify in their properly folded and modified conformations.

Cell-SELEX uses whole living cells as the target, whereby aptamers that recognize specific molecules in their native conformation in their natural environment on the surface of intact cells are selected by repeated amplification and binding to living cells. Thus, Cell-SELEX reflects a more physiological condition because the protein is displayed on the cell surface, including its post-translational modifications, rather than as an isolated and purified protein. In this cell-based selection, specific cell surface molecules/receptors, even unknown, can be directly targeted within their native environment, allowing a straightforward enrichment of cell-specific aptamers. Cell-SELEX generally consists of 2 procedures: positive selection with the target cells, and negative selection with non-targeted cells. Therefore, the specificity and affinity of aptamers essentially relies upon the differences between 2 types of cells or different states of a cell, which also makes it possible to simultaneously enrich for aptamers against several membrane receptors.

Cell surface proteins cycle intra-cellularly to some extent, and many surface receptors are actively internalized in response to ligand binding. For example, the glucose transporter GLUT4 is internalized by adipocytes through clathrin- and caveolin-mediated pathways. Therefore, aptamers that bind to cell surface receptors may be exploited for the delivery of a variety of cargos into cells. As a result, Cell-SELEX is used in the compositions and methods of the invention to identify aptamers that can drive the selective delivery of the miRNA analogs to the targeted human cells (for example, ATMSCs and adipocytes).

Briefly, the selection of aptamers by Cell-SELEX starts with a library of single-stranded DNA and modified RNA nucleic acids that contain an approximately 40 to 60-mer random sequence region flanked by two approximately 20-mer PCR primer sequences. The library is incubated with the live and intact target ATMSCs and adipocyte cells to allow binding to take place. Then the cells are washed and the nucleic acid sequences bound to the cell surface are eluted. The collected sequences are then allowed to interact with excess negative control cells, and only the nucleic acid sequences that remain free in the supernatant are collected and amplified for the next round selection. The subtraction process efficiently eliminates the nucleic acid sequences that are bound to the control cells, while those target-cell-specific aptamer candidates are enriched. After multi-round selection (usually about 10 to 20 rounds achieve excellent enrichment of aptamer candidates), the highly enriched aptamer pools are cloned and sequenced by a high-throughput Next Generation Sequencing (NGS) method.

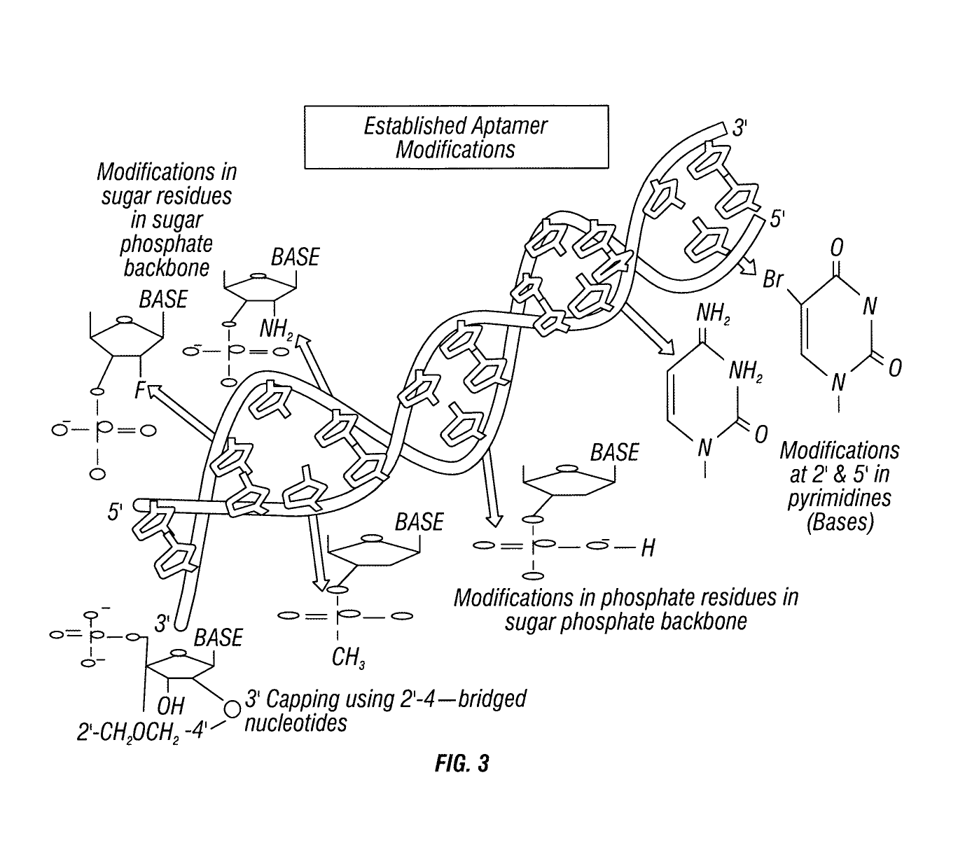

Further optimization for both large-scale synthesis and in vivo applications is achieved through a progressive set of modifications. Various modifications can be made to an aptamer to increase its stability and functionality, such as one or more modifications at the sugar, base, or internucleotide linkage, as shown in FIG. 3. These modifications include, for example, 5′- and 3′-terminal and internal deletions to reduce the size of the aptamer, reselection for sequence modifications that increase the affinity or efficiency of target binding, introduction of stabilizing base-pair changes that increase the stability of helical elements in the aptamer, site-specific modifications of the 2′-ribose and phosphate positions to increase thermodynamic stability and to block nuclease degradation in vivo, and the addition of 5′- and/or 3′-caps to block degradation by exonucleases. For example, pyrimidine bases may be modified at the 5th position with iodide (I), bromide (Br), chloride (Cl), amino (NH₃), azide (N₃) to enhance the stability of the aptamer. Also by way of example, sugar residues may be modified at the 2′ position with amino (NH₂), fluoro (F), and methoxy (OCH₃) groups. Other modifications include substitution of 4-thiouridine, substitution of 5-bromo or 5-iodouracil, backbone modifications, methylations, unusual base pairing combinations such as isobases, isocytidine, and isoguanodine, and 3′ capping. Aptamers generated through these optimizations are typically 15 to 40 nucleotides long and exhibit serum half-lives greater than 10 hours.

In certain embodiments, the aptamer element may include Locked Nucleic Acid (LNA) bases or thiophosphate modifications. The incorporation of LNA bases (methylene link between the 2′oxygen and 4′carbon of the ribose ring) into a stem-loop structure has been shown to increase the melting temperature, nuclease stability and overall stability of the secondary structure of aptamers. Thiophosphate-modified aptamers (thioaptamers) bind to target proteins with high affinity (Kd in nM range) and specificity, and are characterized by a) enhanced nuclease resistance, b) easy synthesis and chemical modification, and c) lack of immunogenicity. Such modifications may be desirable in certain applications.

### D. METHODS OF TREATMENT

In some embodiments, provided herein are methods for delivering a therapeutic microRNA (miRNA) to a specific cell, tissue, or organ in a subject and methods for treating obesity or metabolic disorders in a subject. The method generally comprises administering to the human subject an effective amount of a miRNA agent that modulates activity of at least one thermogenic regulator, (e.g., a mitochondrial uncoupler, such as UCP1 and/or UCP2).

Such methods of treatment may be specifically tailored or modified, based on knowledge obtained from the field of pharmacogenomics. Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Aptamirs and exomirs can be tested in an appropriate animal model e.g., an obesity model including ob/ob mice (Lindstrom, 2007) and db/db mice (Sharma et al., 2003). For example, an aptamir/exomir as described herein can be used in an animal model to determine the efficacy, toxicity, or side effects of treatment with said agent. Alternatively, a therapeutic agent can be used in an animal model to determine the mechanism of action of such an agent. For example, a miRNA agent can be used in an animal model to determine the efficacy, toxicity, or side effects of treatment with such an agent. Alternatively, an agent can be used in an animal model to determine the mechanism of action of such an agent.

The disclosure also provides a method of inducing pre-adipocytes to differentiate into white adipocytes and white adipocytes into brown adipocytes, comprising administering to a population of pre-adipocytes one or more miRNAs disclosed herein.

The disclosure also provides a method for increasing insulin sensitivity in a subject in need thereof comprising administering the subject one or more miRNAs disclosed herein.

The disclosure also provides a method of causing fat loss in a subject in need thereof comprising administering the subject one or more miRNAs disclosed herein.

A miRNA analog (within an aptamir or exomir) modified for enhance uptake into cells (e.g., adipose cells) can be administered at a unit dose less than about 15 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of miRNA analog (e.g., about 4.4.times.1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA silencing agent per kg of bodyweight. The unit dose, for example, can be administered by injection (e.g., intravenous or intramuscular), an inhaled dose, or a topical application. Particularly preferred dosages are less than 2, 1, or 0.1 mg/kg of body weight.

Delivery of an miRNA analog (within an aptamir or exomir) directly to an organ or tissue (e.g., directly to adipose tissue) can be at a dosage on the order of about 0.00001 mg to about 3 mg per organ/tissue, or preferably about 0.0001-0.001 mg per organ/tissue, about 0.03-3.0 mg per organ/tissue, about 0.1-3.0 mg per organ/tissue or about 0.3-3.0 mg per organ/tissue. The dosage can be an amount effective to treat or prevent obesity. In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In certain embodiment, a subject is administered an initial dose, and one or more maintenance doses of a composition. The maintenance dose or doses are generally lower than the initial dose, e.g., one-half less of the initial dose. A maintenance regimen can include treating the subject with a dose or doses ranging from 0.01 mg/kg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in conditions, e.g., changes in percentage of body fat. The dosage of the compound may either be increased in the event the patient does not respond significantly to current dosage levels, or the dose may be decreased if a decrease in body fat is observed, or if undesired side effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., sub-cutaneous, intravenous, intraperitoneal, intracisternal or intracapsular), or reservoir may be advisable. In one embodiment, a pharmaceutical composition includes a plurality of miRNA agent species. In another embodiment, the miRNA agent species has sequences that are non-overlapping and non-adjacent to another species with respect to a naturally occurring target sequence. In another embodiment, the plurality of miRNA agent species is specific for different naturally occurring target genes. In another embodiment, the miRNA agent is allele specific. In another embodiment, the plurality of miRNA agent species target two or more SNP alleles (e.g., two, three, four, five, six, or more SNP alleles).

Following successful treatment, it may be desirable to have the patient undergo maintenance therapy to prevent the recurrence of the disease state, wherein the compound of the invention is administered in maintenance doses, ranging from 0.01 mg per kg to 100 mg per kg of body weight (see U.S. Pat. No. 6,107,094).

The “effective amount” of the miRNA analog (within an aptamir) is an amount sufficient to be effective in treating or preventing a disorder or to regulate a physiological condition in humans. The concentration or amount of miRNA agent administered will depend on the parameters determined for the agent and the method of administration, e.g. nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a composition of the invention can include a single treatment or, preferably, can include a series of treatments. It will also be appreciated that the effective dosage of composition for treatment may increase or decrease over the course of a particular treatment. Changes in dosage may result and become apparent from the results of diagnostic assays as described herein. For example, the subject can be monitored after administering a miRNA analog (within an aptamir). Based on information from the monitoring, an additional amount of the miRNA analog (within an aptamir) can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene, e.g., a gene that produces a target mRNA (e.g., a thermogenic regulator). The transgenic animal can be deficient for the corresponding endogenous mRNA. In another embodiment, the composition for testing includes a miRNA analog that is complementary, at least in an internal region, to a sequence that is conserved between a nucleic acid sequence in the animal model and the target nucleic acid sequence in a human.

Several studies have reported successful mammalian dosing using miRNA agents. For example, Esau, et al., 2006 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST 3/4 45, ALT 3/4 35) for all doses with the exception of the 75 mg/kg dose of miR-122 ASO, which showed a very mild increase in ALT and AST levels. They concluded that 50 mg/kg was an effective, nontoxic dose. Another study by Krutzfeldt, et al., 2005, injected antagomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen, et al., 2008, report that efficient silencing of miR-122 was achieved in primates by three doses of 10 mg per kg LNA-antimiR, leading to a long-lasting and reversible decrease in total plasma cholesterol without any evidence for LNA-associated toxicities or histopathological changes in the study animals.

The compositions of the invention may be directly introduced into a cell (e.g., an adipocyte); or introduced extra-cellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

In certain embodiments, the methods described herein include co-administration of miRNA agents with other drugs or pharmaceuticals, e.g., compositions for modulating thermogenesis, compositions for treating diabetes, compositions for treating obesity. Compositions for modulating thermogenesis include beta-3 adrenergic receptor agonists, thyroid hormones, PPARG agonists, leptin, adiponectin, and orexin.

### E. PHARMACEUTICAL COMPOSITIONS

In one aspect, the methods disclosed herein can include the administration of pharmaceutical compositions and formulations comprising miRNAs associated with aptamers (aptamirs) or encapsulated in exosomes (exomirs) capable of modulating the activity of at least one thermogenic modulator.

In certain embodiments, the compositions of the invention are formulated with a pharmaceutically acceptable carrier. The pharmaceutical compositions and formulations can be administered parenterally, topically, by direct administration into the gastrointestinal tract (e.g., orally or rectally), or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy. 21st ed., 2005.

The aptamirs or exomirs can be administered alone or as a component of a pharmaceutical formulation (composition). The aptamirs or exomirs may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, topical, parenteral, and/or intravenous administration. The formulations may conveniently be presented in unit dosage form and may be prepared by any methods well known in the art of pharmacy. The amount of active ingredient (e.g., miRNA analogs) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations of the invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragés, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragé cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In certain embodiments, oil-based pharmaceuticals are used for administration of the miRNA agents. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, et al., 1997.

In certain embodiments, the pharmaceutical compositions and formulations are in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

In certain embodiments, the pharmaceutical compositions and formulations are administered by in intranasal, intraocular and intravaginal routes including suppositories, insufflation, powders and aerosol formulations (for examples of steroid inhalants, see e.g., Rohatagi, 1995; Tjwa, 1995). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In certain embodiments, the pharmaceutical compositions and formulations are delivered transdermally, by a topical route, formulated as applicator sticks, solutions, suspensions, emulsions, gels, creams, ointments, pastes, jellies, paints, powders, and aerosols.

In certain embodiments, the pharmaceutical compositions and formulations are delivered as microspheres for slow release in the body. For example, microspheres can be administered via intradermal injection of drug which slowly release subcutaneously; see Rao, 1995; as biodegradable and injectable gel formulations, see, e.g., Gao, 1995; or, as microspheres for oral administration, see, e.g., Eyles, 1997.

In certain embodiments, the pharmaceutical compositions and formulations are parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well-known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In certain embodiments, the pharmaceutical compounds and formulations are lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL nucleic acid, about 15 mg/mL sucrose, about 19 mg/mL NaCl, and a sodium citrate buffer having a pH greater than 5.5 but less than 6.5. See, e.g., U.S. 20040028670.

The formulations of the invention can be administered for prophylactic and/or therapeutic treatments. In certain embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in certain embodiments, pharmaceutical compositions of the invention are administered in an amount sufficient to treat obesity in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones, 1996; Groning, 1996; Fotherby, 1996; Johnson, 1995; Rohatagi, 1995; Brophy, 1983; Remington: The Science and Practice of Pharmacy, 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate. Single or multiple administrations of formulations can be given depending on for example: the dosage and frequency as required and tolerated by the patient, the degree and amount of cholesterol homeostasis generated after each administration, and the like. The formulations should provide a sufficient quantity of active agent to effectively treat, prevent or ameliorate conditions, diseases or symptoms, e.g., treat obesity.

In certain embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

### F. EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

Furthermore, in accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### Example 1

### In-Silico Analysis of Thermogenic Regulators

Ninety-two proteins that are involved in the regulation of thermogenesis were selected based upon a critical assessment and review of the available scientific information and the experimental data. These proteins were categorized as activators or repressors of thermogenesis based upon their function(s). These thermogenic regulator proteins are set forth in Table 2. These thermogenic regulator proteins were utilized to identify putative thermogenic miRNAs with in silico tools.

TABLE 2
Thermogenic regulator proteins

| | | |
|---|---|---|
| Name | Entrez Gene ID | Ensembl Gene ID |

Activators

| | | | |
|---|---|---|---|
| 1 | ALDH1A1 | 216 | ENSG00000165092 |
| 2 | AZGP1 | 563 | ENSG00000160862 |
| 3 | BMP7 | 655 | ENSG00000101144 |
| 4 | BMP8b | 656 | ENSG00000116985 |
| 5 | CEBPA | 1050 | ENSG00000245848 |
| 6 | CEBPB | 1051 | ENSG00000172216 |
| 7 | CEBPD | 1052 | ENSG00000221869 |
| 8 | CIDEA | 1149 | ENSG00000176194 |
| 9 | COX7A1 | 1346 | ENSG00000161281 |
| 10 | CRAT | 1384 | ENSG00000095321 |
| 11 | CREB1 | 1385 | ENSG00000118260 |
| 12 | CREBBP | 1387 | ENSG00000005339 |
| 13 | CTBP1 | 1487 | ENSG00000159692 |
| 14 | CTBP2 | 1488 | ENSG00000175029 |
| 15 | DIO2 | 1734 | ENSG00000211448 |
| 16 | EBF2 | 64641 | ENSG00000221818 |
| 17 | ELOVL3 | 83401 | ENSG00000119915 |
| 18 | FGF16 | 8823 | ENSG00000196468 |
| 19 | FGF19 | 9965 | ENSG00000162344 |
| 20 | FGF21 | 26291 | ENSG00000105550 |
| 21 | FNDC5 | 252995 | ENSG00000160097 |
| 22 | FOXC2 | 2303 | ENSG00000176692 |
| 23 | GDF3 | 9573 | ENSG00000184344 |
| 24 | HCRT (OREXIN) | 3060 | ENSG00000161610 |
| 25 | HOXC8 | 3224 | ENSG00000037965 |
| 26 | INSR | 3643 | ENSG00000171105 |
| 27 | IRS1 | 3667 | ENSG00000169047 |
| 28 | KDM3A (JMJD1A) | 55818 | ENSG00000115548 |
| 29 | KLF4 | 9314 | ENSG00000136826 |
| 30 | KLF5 | 688 | ENSG00000102554 |
| 31 | KLF6 | 1316 | ENSG00000067082 |
| 32 | KLF11 | 8462 | ENSG00000172059 |
| 33 | KLF15 | 28999 | ENSG00000163884 |
| 34 | LRP6 | 4040 | ENSG00000070018 |
| 35 | MAPK14 | 1432 | ENSG00000112062 |
| 36 | MED13 | 9969 | ENSG00000108510 |
| 37 | NCOA1 | 8648 | ENSG00000084676 |
| 38 | NCOA2 | 10499 | ENSG00000140396 |
| 39 | NCOA3 | 8202 | ENSG00000124151 |
| 40 | NPPA (ANP) | 4878 | ENSG00000175206 |
| 41 | NR4A3 | 8013 | ENSG00000119508 |
| 42 | NRF1 | 4899 | ENSG00000106459 |
| 43 | PLAC8 | 51316 | ENSG00000145287 |
| 44 | PPARA | 5465 | ENSG00000186951 |
| 45 | PPARD | 5467 | ENSG00000112033 |
| 46 | PPARG | 5468 | ENSG00000132170 |
| 47 | PPARGC1A | 10891 | ENSG00000109819 |
| 48 | PPARGC1B | 133522 | ENSG00000155846 |
| 49 | PRDM16 | 63976 | ENSG00000142611 |
| 50 | PRDX3 | 10935 | ENSG00000165672 |
| 51 | PRKAA1 (AMPKA1) | 5562 | ENSG00000132356 |
| 52 | PRKAA2 (AMPKA2) | 5563 | ENSG00000162409 |
| 53 | PRKACA | 5566 | ENSG00000072062 |
| 54 | PRKACB | 5567 | ENSG00000142875 |
| 55 | PRKG1 | 5592 | ENSG00000185532 |
| 56 | PRKAR1A | 5573 | ENSG00000108946 |
| 58 | SIRT1 | 23411 | ENSG00000096717 |
| 58 | SIRT3 | 23410 | ENSG00000142082 |
| 59 | SLC27A2 (FATP2) | 11001 | ENSG00000140284 |
| 60 | SREBF1 | 6720 | ENSG00000072310 |
| 61 | SREBF2 | 6721 | ENSG00000198911 |
| 62 | STAT5A | 6776 | ENSG00000126561 |
| 63 | TRPM8 | 79054 | ENSG00000144481 |
| 64 | UCP1 (SLC25A7) | 7350 | ENSG00000109424 |
| 65 | UCP2 (SLC25A8) | 7351 | ENSG00000175567 |
| 66 | UCP3 (SLC25A9) | 7352 | ENSG00000175564 |

Repressors

| | | | |
|---|---|---|---|
| 1 | ATG7 | 10533 | ENSG00000197548 |
| 2 | BMP2 | 650 | ENSG00000125845 |
| 3 | BMP4 | 652 | ENSG00000125378 |
| 4 | CIDEC | 63924 | ENSG00000187288 |
| 5 | CTNNB1 | 1499 | ENSG00000168036 |
| 6 | DLK1 (Pref-1) | 8788 | ENSG00000185559 |
| 7 | E2F4 (p107) | 1874 | ENSG00000205250 |
| 8 | EIF4EBP1 | 1978 | ENSG00000187840 |
| 9 | ESRRA (NR3B1) | 2101 | ENSG00000173153 |
| 10 | GATA2 | 2624 | ENSG00000179348 |
| 11 | GATA3 | 2625 | ENSG00000107485 |
| 12 | IKBKE | 9641 | ENSG00000143466 |
| 13 | KLF2 | 10365 | ENSG00000127528 |
| 14 | KLF7 | 8609 | ENSG00000118263 |
| 15 | NR1H3 (LXRA) | 10062 | ENSG00000025434 |
| 16 | NRIP1 (RIP140) | 8204 | ENSG00000180530 |
| 17 | RBI (pRb) | 5925 | ENSG00000139687 |
| 18 | NR0B2 (SHP) | 8431 | ENSG00000131910 |
| 19 | RPS6KB1 | 6198 | ENSG00000108443 |
| 20 | RUNX1T1 | 862 | ENSG00000079102 |
| 21 | RUNX2 | 860 | ENSG00000124813 |
| 22 | TNFRSF1A | 7132 | ENSG00000067182 |
| 23 | TRPV4 | 59341 | ENSG00000111199 |
| 24 | TWIST1 | 7291 | ENSG00000122691 |
| 25 | WNT5A | 7474 | ENSG00000114251 |
| 26 | WNT10B | 7480 | ENSG00000169884 |

The STRING 9.0 database of known and predicted protein interactions (string-db.org/) was used to test these 92 candidate molecules. The interactions include direct (physical) and indirect (functional) associations; they are derived from four sources: genomic context; high-throughput experiments; co-expression; and previous knowledge. STRING quantitatively integrates interaction data from these sources for a large number of organisms, and transfers information between these organisms where applicable. The database currently covers 5,214,234 proteins from 1,133 organisms. As an example, the relationships between the 92 thermogenic regulator molecules were centered on UCP1, and molecules having direct and indirect connections with UCP1 could be distinguished using a high confidence score of 0.90. From this analysis, it was discovered that nine molecules (CEBPB, CIDEA, KDM3A, NRIP1, PRDM16, PPARG, PPARGC1A, PPKAA2, and UCP2) are directly linked to UCP1, whereas many more molecules are connected to UCP1 on a second or higher degree order.

Similarly, the interactions among these 92 thermogenic regulator molecules were independently assessed using the commercially available Ingenuity Pathway Analysis (IPA) Software program and the Reactome Functional Interaction (Reactome IF) Software program, each of which are available online. The IPA and Reactome IF networks differ from the one obtained with the STRING program. It is not surprising that the results of these algorithms are different because they rely on different predefined parameters, sources of information and selection criteria. See International Application Serial No. PCT/US2013/037579 filed on Apr. 22, 2013, incorporated herein by reference in its entirety.

### Example 2

### In-Silico Selection of Relevant miRNA Candidates

To select thermogenic miRNA analogs, thirty four internet-based resources were employed to match miRNAs and their targets (the “micronome”).

Specifically, these tools were used to perform: 1) Integrated Data Mining (8 tools); 2) miRNA Mining and Mapping (6 tools); 3) miRNA Target Targets and Expression (21 tools); 4) Integrated miRNA Targets and Expression (13 tools); 5) miRNA Secondary Structure Prediction and Comparison (5 tools); 6) Network Searches and Analyses (8 tools); 7) Molecular Visualization (4 tools); and 8) Information Integration and Exploitation (1 tool).

A single gene target can be controlled by several miRNAs whereas a single miRNA can control several gene targets. Sophisticated bioinformatics resources have been developed to select the most relevant miRNAs to target diseases (Gallagher, et al., 2010; Fujiki, et al., 2009; Okada, et al., 2010; Hao, et al., 2012; Hao, et al., 2012). However, the results of these algorithms are acutely dependent on predefined parameters and the degree of convergence between these algorithms is rather limited. Therefore, there is a need to develop better performing bioinformatics tools with improved sensitivity, specificity and selectivity for the identification of miRNA/target relationships.

Consequently, 8 score matrices predicted by DianaMicroT, miRWalk, picTar4way, picTar5way, PITA, RNA22, TargetScan conserved and TargetScan non conserved, were created as well as and 4 matrices of miRNA-gene interactions verified experimentally which were extracted from the following tools: Starbase, miRWalk, miRTarBase and miRRecords. These matrices have 19,061 rows (genes) and 2,043 columns (mature miRNAs). The data were fused to form a single score matrix summarizing all such information, which is used for all ranking procedures. Queries can be either rank all genes given a certain miRNA, or rank all miRNAs given a certain gene. The data fusion method consists in first applying logistic regression to predict the All Verified Merged values from the 8 score values, then applying singular value decomposition and reconstruction of the score matrix to improve the resulting scores. Performance was assessed first by training using half of the verified values (complemented by an equal number of non verified values treated as negative outcomes). Validation testing on all the remaining values reached an ROC AUC of 0.91.

The interactions between miRNAs and their targets go beyond the original description of miRNAs as post-transcriptional regulators whose seed region of the driver strand (5′ bases 2-7) bind to complementary sequences in the 3′ UTR region of target mRNAs, usually resulting in translational repression or target degradation and gene silencing. The interactions can also involve various regions of the driver or passenger strands of the miRNAs as well as the 5′UTR, promoter, and coding regions of the mRNAs.

Upon analysis of the available data, it was decided to favor pathway-specific miRNAs which target multiple genes within one discrete signaling pathway, rather than universal miRNAs which are involved in many signaling pathways, functions or processes in various cell types and tissues/organs. Using these 34 publicly available Internet tools predicting miRNAs and their targets, specific human miRNAs were searched for that could potentially modulate several targets among the 92 thermogenic regulator molecules (which include 36 Transcription Factors) selected in Example 1.

Once the lists of miRNAs of interest and their mRNA targets were produced, the following filters were applied to refine the results:

Filters:

1. Expression of miRNAs in tissue/cell of interest
2. Number of algorithms predicting one miRNA for a given gene or set of genes
3. Score/percent from algorithms
4. Number of preferred genes targeted by one miRNA
5. Number of binding sites in a target gene for one miRNA
6. Number of binding sites in a target gene for several miRNAs
7. Over-representation of one miRNA seed complementary sequence among target genes (miRvestigator)
8. Validated miRNA-mRNA target couples
9. Genomic location of miRNA binding site (5′UTR-Promoter-CDS-3′UTR)
10. Intronic location of miRNA
11. Clustering of miRNAs
12. Abundance of miRNA in specific tissue/cell of interest

Applying the above parameters, it was discovered that 229 miRNAs met at least two of these criteria. These miRNAs are ranked according to decreasing number of selection criteria (≧2): hsa-miR-20b-5p; hsa-miR-27b-3p; hsa-miR-103a-3p; hsa-miR-22-3p; hsa-miR-34a-5p; hsa-miR-130b-3p; hsa-miR-132-3p; hsa-miR-181b-5p; hsa-miR-211-5p; hsa-miR-148b-3p; hsa-miR-17-5p; hsa-miR-182-5p; hsa-miR-20a-5p; hsa-miR-27a-3p; hsa-miR-301a-3p; hsa-miR-204-5p; hsa-miR-143-3p; hsa-miR-1; hsa-miR-9-5p; hsa-miR-30a-5p; hsa-miR-138-5p; hsa-miR-217; hsa-miR-19b-3p; hsa-miR-382-5p; hsa-miR-106a-5p; hsa-miR-107; hsa-miR-135a-5p; hsa-miR-93-5p; hsa-miR-21-5p; hsa-miR-515-3p; hsa-miR-106b-3p; hsa-miR-125a-5p; hsa-miR-148a-3p; hsa-miR-155-5p; hsa-miR-181a-5p; hsa-miR-519d; hsa-miR-96-5p; hsa-miR-212-3p; hsa-miR-29a-3p; hsa-miR-98-5p; hsa-let-7c; hsa-let-7d-5p; hsa-miR-141-3p; hsa-miR-183-5p; hsa-miR-19a-3p; hsa-miR-196a-5p; hsa-miR-30b-5p; hsa-miR-378a-3p; hsa-miR-302c-5p; hsa-miR-30e-5p; hsa-miR-130a-3p; hsa-let-7e-5p; hsa-miR-216a-5p; hsa-miR-450a-5p; hsa-let-7d-3p; hsa-miR-26b-5p; hsa-miR-181c-5p; hsa-miR-186-5p; hsa-miR-519c-3p; hsa-let-7b-5p; hsa-miR-10b-5p; hsa-miR-125b-5p; hsa-miR-134; hsa-miR-137; hsa-miR-150-5p; hsa-miR-153; hsa-miR-15b-5p; hsa-miR-16-5p; hsa-miR-195-5p; hsa-miR-196b-5p; hsa-miR-23a-3p; hsa-miR-29c-3p; hsa-miR-373-3p; hsa-miR-7-5p; hsa-miR-214-3p; hsa-miR-421; hsa-miR-15a-5p; hsa-miR-193b-3p; hsa-miR-194-5p; hsa-miR-223-3p; hsa-miR-30d-5p; hsa-miR-424-5p; hsa-miR-454-3p; hsa-miR-545-3p; hsa-miR-485-5p; hsa-miR-335-5p; hsa-miR-133a; hsa-miR-222-3p; hsa-miR-494; hsa-miR-498; hsa-miR-513a-5p; hsa-miR-92a-3p; hsa-miR-495-3p; hsa-miR-503-5p; hsa-miR-539-5p; hsa-miR-16-2-3p; hsa-miR-302b-5p; hsa-miR-425-3p; hsa-miR-99a-3p; hsa-let-7a-3p; hsa-miR-126-3p; hsa-miR-20a-3p; hsa-miR-499a-5p; hsa-let-7g-5p; hsa-miR-152; hsa-miR-26a-5p; hsa-miR-124-3p; hsa-miR-203a; hsa-miR-24-3p; hsa-miR-301b; hsa-miR-590-3p; hsa-miR-1179; hsa-miR-325; hsa-miR-552; hsa-miR-185-5p; hsa-miR-455-3p; hsa-miR-583; hsa-miR-122-5p; hsa-miR-1305; hsa-miR-139-5p; hsa-miR-146a-5p; hsa-miR-18a-5p; hsa-miR-18b-5p; hsa-miR-199b-5p; hsa-miR-340-5p; hsa-miR-34c-5p; hsa-miR-423-3p; hsa-miR-489; hsa-miR-520f; hsa-miR-520g; hsa-miR-605; hsa-miR-668; hsa-let-7a-5p; hsa-let-7f-5p; hsa-miR-110a-3p; hsa-miR-135b-5p; hsa-miR-144-3p; hsa-miR-181d; hsa-miR-200b-3p; hsa-miR-200c-3p; hsa-miR-218-5p; hsa-miR-23b-3p; hsa-miR-25-3p; hsa-miR-29b-3p; hsa-miR-383; hsa-miR-202-3p; hsa-miR-381-3p; hsa-miR-377-3p; hsa-miR-452-5p; hsa-miR-501-3p; hsa-miR-514a-3p; hsa-miR-654-3p; hsa-let-7b-3p; hsa-miR-125a-3p; hsa-miR-133b; hsa-miR-192-5p; hsa-miR-199a-3p; hsa-miR-30c-5p; hsa-miR-335-3p; hsa-miR-374a-5p; hsa-miR-410; hsa-miR-429; hsa-miR-497-5p; hsa-miR-513a-3p; hsa-miR-542-3p; hsa-miR-653; hsa-miR-122-3p; hsa-miR-101-5p; hsa-miR-1178-3p; hsa-miR-191-5p; hsa-miR-214-5p; hsa-miR-302d-5p; hsa-miR-572; hsa-miR-574-3p; hsa-miR-26a-2-3p; hsa-miR-611; hsa-let-7f-1-3p; hsa-let-7i-3p; hsa-miR-100-5p; hsa-miR-106b-5p; hsa-miR-132-5p; hsa-miR-135b-3p; hsa-miR-136-3p; hsa-miR-150-3p; hsa-miR-154-3p; hsa-miR-15a-3p; hsa-miR-15b-3p; hsa-miR-16-1-3p; hsa-miR-181a-2-3p; hsa-miR-181c-3p; hsa-miR-186-3p; hsa-miR-195-3p; hsa-miR-20b-3p; hsa-miR-223-5p; hsa-miR-224-3p; hsa-miR-24-1-5p; hsa-miR-24-2-5p; hsa-miR-27a-5p; hsa-miR-27b-5p; hsa-miR-29b-1-5p; hsa-miR-302a-5p; hsa-miR-3065-5p; hsa-miR-30d-3p; hsa-miR-34a-3p; hsa-miR-371a-3p; hsa-miR-373-5p; hsa-miR-374a-3p; hsa-miR-376a-5p; hsa-miR-378a-5p; hsa-miR-424-3p; hsa-miR-451a; hsa-miR-452-3p; hsa-miR-487b; hsa-miR-493-5p; hsa-miR-500a-3p; hsa-miR-502-3p; hsa-miR-516b-3p; hsa-miR-518e-3p; hsa-miR-518f-3p; hsa-miR-519a-5p; hsa-miR-519b-5p; hsa-miR-521; hsa-miR-523-5p; hsa-miR-545-5p; hsa-miR-585; hsa-miR-7-2-3p; hsa-miR-93-3p; hsa-miR-96-3p; and hsa-miR-99b-3p.

### Example 3

### High-Throughput miRNA Target Screening by Luciferase Activity and qRT-PCR

High-throughput screening using luciferase reporter assay constructs are used to identify novel miRNA targets involved in thermogenesis.

Luciferase is commonly used as a reporter to assess the transcriptional activity in cells that are transfected with a genetic construct containing the luciferase gene under the control of a promoter of interest. SwitchGear Genomics has created a genome-wide library of over 18,000 human promoters and 12,000 human 3′ UTR regions cloned into an optimized luciferase reporter vector system containing SwitchGear's RenSP reporter cassette (GoClone™) as a component of the LightSwitch™ Luciferase Assay System. This modified form of luciferase greatly facilitates detailed kinetic studies, especially those focusing on repression, which might otherwise be obscured by reporter protein accumulation.

The multiple microRNAs-one mRNA paradigm was tested with the SwitchGear Genomic GoClone system, using UCP1 as the single thermogenic target gene. In order to explore the possible interactions between various human miRNAs and the 3′UTR region, the 5′UTR region and the promoter/enhancer region of the human UCP1 gene in Hela and HepG2 cells, three reporter constructs were made:
(1) A human UCP1 3′UTR construct containing a reporter gene driven by a strong constitutive promoter (RPL10-prom) with a 2,218 bp 3′UTR fragment of the human UCP1 sequence cloned in the 3′UTR region of the reporter gene. The effects of a specific miRNA mimic, inhibitor, or non-targeting control on this reporter's activity are compared to those of an empty-3′UTR and an Actin Beta-3′UTR to identify effects that are specific to the putative UCP1 3′UTR construct.
(2) A human UCP1 Promoter construct containing a reporter gene driven by a 4,147 bp 5′UTR fragment of the human UCP1 sequence that spans the Transcription Start Site and upstream region covering the methylation region and the enhancer region of the human UCP1 gene sequence. The effects of a specific miRNA mimic, inhibitor, or non-targeting control on this reporter's activity are compared to those of an Actin Beta-Promoter to identify effects that are specific to the putative UCP1 5′UTR construct.
(3) A human UCP1 Enhancer Region construct containing a reporter gene driven by a short minimal promoter from the HSV-TK locus with a 601 bp 5′UTR fragment of the human UCP1 sequence that spans the Enhancer Region of the human UCP1 gene sequence. The effects of a specific miRNA mimic, inhibitor, or non-targeting control on this reporter's activity are compared to those of an empty 5′Enhancer Region to identify effects that are specific to the putative UCP1 5′Enhancer construct.

In addition, miRNAxxx_{—}3′UTR constructs were made. They contain the reporter gene driven by a strong promoter (RPL10_prom) with a perfect match to the target sequence of miRNAxxx cloned into the 3′UTR region of the reporter gene. The effect of a miRNA mimic, inhibitor, or non-targeting control on this reporter's activity can be compared to EMPTY_{—}3′UTR and Actin B_{—}3′UTR to determine whether a miRNA mimic's or inhibitor's activity can be reasonably detected in the experimental cell type. If the cell type has no endogenous expression of the miRNA in question, the addition of a mimic should knock down the activity of this reporter, and the addition of an inhibitor should have no significant effect. If the cell type has high endogenous expression of the miRNA in question, the addition of an inhibitor should increase the activity of this reporter, and the addition of a mimic should have no significant effect. The range of endogenous miRNA expression in Hela and HepG2 cell types is broad, so the synthetic target activity changes are likely to reflect this variability.

For each miRNA candidate (38 in total), the following conditions were tested:
miRNA mimic (specific)*8 reporter constructs in Hela cells
miRNA mimic (specific)*8 reporter constructs in HepG2 cells
miRNA mimic non-targeting control*8 reporter constructs in Hela cells
miRNA mimic non-targeting control*8 reporter constructs in HepG2 cells
miRNA inhibitor (specific)*8 reporter constructs in Hela cells
miRNA inhibitor (specific)*8 reporter constructs in HepG2 cells
miRNA inhibitor non-targeting control*8 reporter constructs in Hela cells
miRNA inhibitor non-targeting control*8 reporter constructs in HepG2 cells

To the extensive list of miRNAs that may bind to the UCP1 sequence, 10 filters were applied (in addition to required binding to UCP1 3′UTR region) to reduce the number of miRNA candidates to be tested. These filters were length of binding sites, number of binding sites, binding to the 5′UTR region, chromosomal clustering with other miRNAs, intronic location, wobbling, expression across species, binding to the Enhancer Region, binding to the Methylation Region and proof of experimental evidence of a relation to UCP1. 38 miRNAs that met at least 3 of these criteria were tested (Table 3).

TABLE 3
miRNA with putative binding sites in the human UCP1 gene sequence

| | | # of | Binding | # of | | | Chr. | | | Inter- | Enhancer | Methylation | Exp. |
|---|---|---|---|---|---|---|---|---|---|---|---|---|---|
| | miRNA | criteria | length | sites | 3′UTR | 5′UTR | Clusters | Intronic | Wobbling | species | Region | Region | Evidence |
| 1 | hsa-miR-130b-5p | 7 | 11 | 3 | + | + | 22 | | | | + | + | + |
| 2 | hsa-miR-328 | 6 | 10 | 4 | + | + | | | | | + | + | + |
| 3 | hsa-miR-655 | 6 | 10 | 5 | + | + | 14 | | | | | + | + |
| 4 | hsa-miR-19b-2-5p | 5 | 10 | 4 | + | + | X | | + | | | | + |
| 5 | hsa-miR-26a-2-3p | 5 | 10 | 7 | + | + | | | | | | + | + |
| 6 | hsa-miR-367-3p | 5 | 10 to 18 | 3 | + | + | 4 | | + | + | + | | |
| 7 | hsa-miR-371a-5p | 5 | 10 to 12 | 9 | + | + | 19 | | | + | | | + |
| 8 | hsa-miR-377-3p | 5 | 10 to 14 | 5 | + | + | 14 | | | + | | | + |
| 9 | hsa-miR-378a-3p | 5 | 7 to 13 | 19 | + | + | | + | + | + | | | + |
| 10 | hsa-miR-382-3p/5p | 5 | 15 | 2 | + | + | 14 | | + | + | | | |
| 11 | hsa-miR-421 | 5 | 10 | 5 | + | + | X | | | | | | + |
| 12 | hsa-miR-515-3p | 5 | 9 | 3 | + | + | 19 | | | | + | | + |
| 13 | hsa-miR-620 | 5 | 10 | 7 | + | + | | | | | + | | + |
| 14 | hsa-miR-941/2 | 5 | 9 | 5 | + | + | 20 | | | | | + | |
| 15 | hsa-miR-1179 | 4 | 11 | 3 | + | + | 15 | | + | | | | |
| 16 | hsa-miR-1302 | 4 | 10 | 5 | + | + | | | | | | + | |
| 17 | hsa-miR-146a | 4 | 9 to 10 | 8 | + | + | | | | | | | + |
| 18 | hsa-miR-181c | 4 | 9 | 5 | + | + | 19 | | | | | | + |
| 19 | hsa-miR-203 | 4 | 9 | 1 | + | | 14 | | | | | + | + |
| 20 | hsa-miR-331-5p | 4 | 8 to 15 | 6 | + | + | 12 | | + | + | | | |
| 21 | hsa-miR-422a | 4 | 7 to 14 | 6 | + | + | | | + | + | | | + |
| 22 | hsa-miR-452 | 4 | 8 | 7 | + | + | X | | | | | | + |
| 23 | hsa-miR-491-5p | 4 | 10 | 3 | + | + | | | | | | | |
| 24 | hsa-miR-501-3p | 4 | 10 | 2 | + | + | X | | | | | | + |
| 25 | hsa-miR-543 | 4 | 10 to 14 | 4 | + | + | 14 | | + | + | | | |
| 26 | hsa-miR-545 | 4 | 11 | 2 | + | + | X | | | | | | + |
| 27 | hsa-miR-549 | 4 | 13 to 14 | 3 | + | + | | | | + | | | + |
| 28 | hsa-miR-643 | 4 | 10 to 14 | 9 | + | + | | | + | | | | + |
| 29 | hsa-miR-651 | 4 | 10 | 6 | + | + | | | | | | | + |
| 30 | hsa-miR-654-3p | 4 | 8 to 10 | 11 | + | + | 14 | | + | | | | |
| 31 | hsa-miR-21-5p | 3 | 10 to 14 | 2 | + | + | | | + | + | | | + |
| 32 | hsa-miR-211-5p | 3 | 11 | 1 | + | | | | | | | + | + |
| 33 | hsa-miR-22-3p | 3 | 9 | 5 | + | + | | | + | | | | + |
| 34 | hsa-miR-30b-5p | 3 | 10 | 1 | + | | 8 | | | | | | + |
| 35 | hsa-miR-325 | 3 | 7 to 8 | 11 | + | + | | | | | | | + |
| 36 | hsa-miR-362-5p | 3 | 10 | 1 | + | | X | | | | | | + |
| 37 | hsa-miR-504 | 3 | 9 | 2 | + | + | | | | | | + | + |
| 38 | hsa-miR-552 | 3 | 9 | 3 | + | + | | | | | | | + |

In these Luciferase reporter gene assay experiments, a miRNA candidate was considered to interact with UCP1 if both the specific miRNA inhibitor increases the luciferase signal and the specific miRNA mimic decreases the luciferase signal with an Inhibitor/Mimic Ratio≧1.5 and or/a p value <0.05. These selection criteria identify 9 miRNAs (hsa-miR-19b-2-5p, hsa-miR-21-5p, hsa-miR-130b-5p, hsa-miR-211, hsa-miR-325, hsa-miR-382-3p/5p, hsa-miR-543, hsa-miR-515-3p, and hsa-miR-545) (Table 4). A few more barely missed these selection criteria; they are hsa-miR-331-5p, hsa-miR-552, hsa-miR-620, and hsa-miR-1179.

TABLE 4
miRNA identified as regulators of UCP1 gene expression by
luciferase reporter assay in Hela and/or HepG2 cells

| Cell Line(s) | miRNA |
|---|---|
| Hela | hsa-miR-130b-5p |
| Hela + HepG2 | hsa-miR-19b-2-5p |
| HepG2 | hsa-miR-382-3p/5p |
| Hela | hsa-miR-515-3p |
| Hela | hsa-miR-543 |
| HepG2 | hsa-miR-545 |
| Hela + HepG2 | hsa-miR-21-5p |
| Hela | hsa-miR-211-5p |
| Hela + HepG2 | hsa-miR-325 |

Out of these 9 selected miRNAs, 3 appear to bind to the 3 regions of UCP1 which were studied (hsa-miR-21-5p, hsa-miR-211, and hsa-miR-515-3p); 3 appear to bind to 2 regions of UCP1 (hsa-miR-19b-2-5p, hsa-miR-130b-5p, and hsa-miR-325), and 3 bind to a single region of UCP1 (hsa-miR-331-5p, hsa-miR-543, and hsa-miR-545). All but hsa-miR-331-5p appear to bind to the 3′UTR region of UCP1 (Table 5).

TABLE 5
miRNA identified as regulators of UCP1 gene expression by
luciferase reporter assay

| | | UCP1 | UCP1 | UCP1 |
|---|---|---|---|---|
| | miRNA | 3′ UTR | Enhancer | Promoter |
| 1 | hsa-miR-21-5p | X | X | X |
| 2 | hsa-miR-211 | X | X | X |
| 3 | hsa-miR-515-3p | X | X | X |
| 4 | hsa-miR-19b-2-5p | X | | X |
| 5 | hsa-miR-130b-5p | X | X | |
| 6 | hsa-miR-325 | X | X | |
| 7 | hsa-miR-331-5p | | X | |
| 8 | hsa-miR-543 | X | | |
| 9 | hsa-miR-545 | X | | |

Further screening is performed by transfection of the promoter/3′UTR library into human adipocytes or adipose-derived mesenchymal stem cells in cell culture, followed by addition of miRNA agents (e.g., agomirs or antagomirs) to the cell culture. Measurement of luciferase activity and identification of mRNAs is performed 24 hours after transfection and addition of miRNA agents.

In order to confirm the results of the transfection experiments set forth above over a longer time frame, lentiviral transduction experiments are performed using lentiviral vectors containing the miRNA agents of interest (from System Biosciences (SBI) collection of miRNA precursors expressed in the pMIRNA1 SBI vectors allowing the expression of the copGFP fluorescent marker). Specifically, cells containing the promoter/3′UTR library are transduced with lentiviral particles at an MOI of 1:10 and GFP-positive cells are sorted by FACS, according to the supplier's instructions. The level of expression of the mature miRNAs and their targeted mRNAs is assessed at several time points (0, 3, and 6 hr.; 1, 4, and 7 days) by Taqman Quantitative Real-time PCR in control cells (HEK293 cells), Human Adipose-Derived Mesenchymal Stem Cells, Human Subcutaneous Pre-adipocytes, and Human Proliferating Subcutaneous Adipocytes. Pooling of RNAs from 5 different time points after transduction is optionally employed to reduce the complexity of the qRT-PCR based screening approach while preserving the detection sensitivity.

### Example 4

### High-Content Cellular Phenotypic and Genotypic Screening of Thermogenic miRNA Candidates

High-content screening methods were used to screen for novel miRNA agents that modulate the activity of thermogenic regulators (e.g., UCP1 and UCP2). High-content screening is a drug discovery method that uses images of living cells to facilitate molecule discovery. Accordingly, high-content screening methods were used to screen for novel miRNA agents that modulate the activity of thermogenic regulators.

In order to assess the effect of miRNA analogs on human white adipocytes differentiation into brown adipocytes, human subcutaneous pre-adipocytes (SuperLot 0048 from 8 female donors, ZenBio, NC) were plated on Day 0 into 96-well plates and allowed to attach overnight in preadipocyte medium (DMEM/Ham's F-12 (1:1, v/v), HEPES buffer, Fetal bovine serum and Antibiotics). The next day (Day 1), the medium was removed and replaced with differentiation medium-2 (DMEM/Ham's F-12 (1:1, v/v), HEPES buffer, Fetal bovine serum, Biotin, Pantothenate, Human insulin, Dexamethasone, Isobutyl-methylxanthine, Proprietary PPARG agonist and Antibiotics. The cells were allowed to incubate for 7 days at 37° C., 5% CO₂. After 7 days (Day 7), a partial medium exchange was performed with AM-1 adipocyte maintenance medium (DMEM/Ham's F-12 (1:1, v/v), HEPES buffer, Fetal bovine serum, Biotin, Pantothenate, Human insulin, Dexamethasone and Antibiotics). The cells were allowed to incubate for an additional 7 days at 37° C., 5% CO₂. On Day 17, the cells were transfected with miRNA analogs (specific miRIDIAN Mimics and Hairpin Inhibitors, Dharmacon/Thermo Scientific Molecular Biology, CO) using the transfecting agent Dharmafect 4. All treatments were in triplicate. Post transfection, the negative control was maintenance medium only and the positive control was maintenance medium with 100 nM of the PPARG agonist rosiglitazone. After 2 days, medium was removed and replaced with fresh maintenance medium. The maintenance medium then changed every two to three days until the end of the treatment period (Day 30). At the end of the treatment (total of 30 days in culture) cells were processed for Phenotyping and Genotyping Screening.

High Content cellular phenotyping of adipocytes in the presence of controls and miRNA analogs has been described into International Application Serial No. PCT/US2013/037579 filed on Apr. 22, 2013 hereby incorporated by reference in its entirety.

mRNA expression was measured by targeted q-RT-PCR, NanoString and universal RNA-Sequencing. The NanoString technology has the following advantages: it does not require RNA extraction, does not require Reverse Transcription and amplification, employs two very specific ≈50 base-long probes per mRNA, and utilizes unique molecular barcode and single molecule imaging for each gene. Universal RNA-Sequencing (or Deep Sequencing) requires the creation of cDNA libraries, but covers the whole genome in an “agnostic” way.

As an example, Day-30 samples of human adipocytes in maintenance medium (negative control) or treated with rosiglitazone (positive control), or one of the first 191 miRNA analogs of the ranked list of the 229 miRNAs introduced in Example 2 were submitted to NanoString technology to assess the level of expression of 82 thermogenic genes and nine control genes.

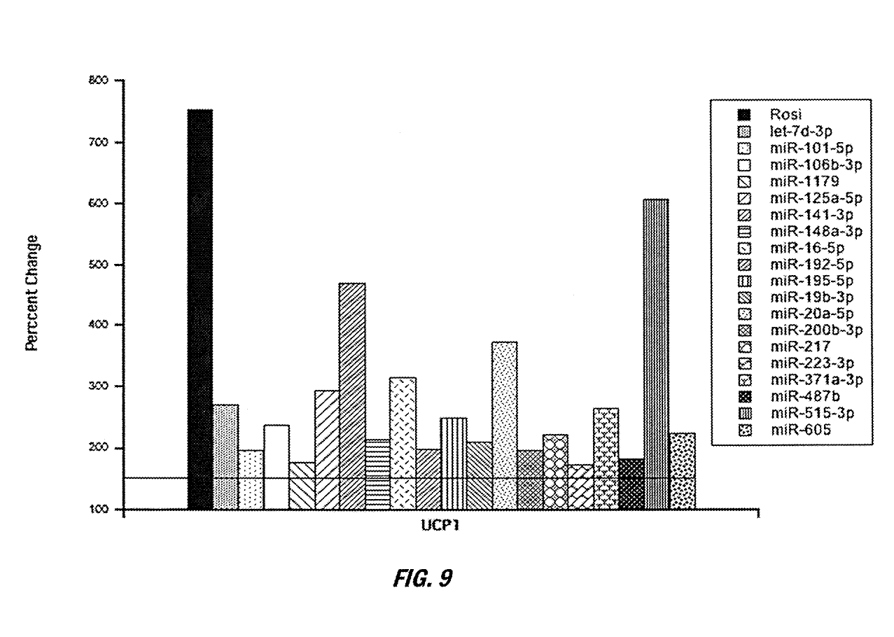

Nineteen miRNA mimics induced at least a 50% increase of the human UCP1 mRNA expression level and 13 miRNA mimics induced at least a two-fold increase of the human UCP1 mRNA expression. It is worthnoting that such alterations were noted 2 weeks after a single transfection with the miRNA analog, whereas the positive control, Rosiglitazone (100 nM) was added with fresh medium every two to three days of culture. (see FIG. 9). Hsa-miR-515-3p which was shown in example 3 above to interact with the human UCP1 gene in the reporter gene assay produced a 604% increase of the human UCP1 mRNA expression, as well as a 183% increase of the human UCP2 mRNA expression. Hsa-miR-141-3p produced 466% and 137% increases of UCP1 and UCP2 mRNAs expression, respectively.

As shown on Table 6, the expression of a few targeted genes increased by at least 25% after exposure to rosiglitazone or a miRNA mimic which produced at least a two-fold increase of the human UCP1 gene expression. Thye include ANP, AZGP1, BMP7, BMP8b, CEBPA, CEBPB, DLK1, FGF19, FGF21, FOXC2, HCRT, KLF11, KLF15, KLF5, MAPK14, NCOA1, NCOA2, NRIP1, PRDX3, PRKAA2, PRKACA, RUNX2, FATP2, and TRPM8. Of note is the absence of expression change for the following mRNAs: FNDC5, MED13, PPARGC1A, PPARGC1B, PRDM16 and UCP3. Table 6 shows the levels of expression of 82 targeted mRNAs after stimulation of human mature subcutaneous adipocytes after exposure to rosiglitazone (RSGLTZ) or one of miRNA mimic.

TABLE 6
Changes in gene expression

| | | Let-7d- | miR- | miR- | miR- | miR- | miR- | miR- | miR- | miR- | miR- | miR- | miR- | miR- |
|---|---|---|---|---|---|---|---|---|---|---|---|---|---|---|
| Gene | RSGITZ | 3p | 106b-3p | 125a-5p | 141-3p | 148a-3p | 16-5p | 195-5p | 19b-3p | 20a-5p | 217 | 371a-3p | 515-3p | 605 |
| ALDH1A1 | 42% | 90% | 101% | 59% | 58% | 97% | 63% | 61% | 51% | 79% | 81% | 62% | 56% | 97% |
| ANP | 94% | 225% | 121% | 158% | 120% | 143% | 104% | 122% | 62% | 161% | 143% | 113% | 168% | 204% |
| (NPPA) | | | | | | | | | | | | | | |
| ATG7 | 83% | 94% | 98% | 95% | 108% | 118% | 60% | 75% | 97% | 98% | 92% | 93% | 99% | 93% |
| AZGP1 | 71% | 146% | 125% | 64% | 299% | 82% | 67% | 73% | 72% | 139% | 99% | 75% | 127% | 133% |
| BMP2 | 86% | 145% | 88% | 100% | 78% | 121% | 86% | 73% | 84% | 164% | 106% | 94% | 115% | 118% |
| BMP4 | 50% | 91% | 59% | 79% | 72% | 71% | 30% | 48% | 53% | 88% | 57% | 75% | 57% | 74% |
| BMP7 | 123% | 315% | 245% | 226% | 259% | 321% | 113% | 214% | 285% | 609% | 214% | 375% | 145% | 320% |
| BMP8b | 104% | 132% | 184% | 216% | 191% | 168% | 84% | 97% | 224% | 315% | 213% | 192% | 117% | 175% |
| CEBPA | 311% | 129% | 117% | 117% | 186% | 226% | 76% | 79% | 124% | 240% | 108% | 173% | 261% | 141% |
| CEBPB | 123% | 83% | 110% | 120% | 140% | 159% | 73% | 74% | 122% | 168% | 139% | 170% | 167% | 160% |
| CEBPD | 46% | 114% | 99% | 101% | 74% | 127% | 109% | 130% | 108% | 77% | 85% | 82% | 71% | 105% |
| CIDEA | 413% | 36% | 57% | 38% | 46% | 50% | 59% | 45% | 51% | 70% | 40% | 46% | 68% | 55% |
| CIDEC | 114% | 90% | 84% | 120% | 170% | 95% | 38% | 46% | 85% | 110% | 94% | 101% | 137% | 88% |
| COX7A1 | 108% | 83% | 95% | 80% | 123% | 86% | 63% | 74% | 96% | 108% | 81% | 91% | 98% | 119% |
| CRAT | 128% | 102% | 106% | 108% | 138% | 115% | 82% | 83% | 108% | 107% | 102% | 102% | 122% | 86% |
| CREB1 | 80% | 99% | 104% | 102% | 91% | 121% | 87% | 93% | 109% | 106% | 103% | 83% | 85% | 95% |
| CREBBP | 72% | 103% | 75% | 78% | 90% | 94% | 103% | 92% | 85% | 65% | 87% | 60% | 74% | 57% |
| CTBP1 | 89% | 87% | 77% | 87% | 86% | 98% | 74% | 70% | 86% | 73% | 96% | 80% | 91% | 70% |
| CTBP2 | 89% | 77% | 100% | 102% | 83% | 146% | 74% | 76% | 105% | 103% | 80% | 113% | 87% | 103% |
| CTNNB1 | 89% | 98% | 96% | 92% | 85% | 97% | 98% | 98% | 104% | 102% | 80% | 96% | 96% | 95% |
| DIO2 | 88% | 73% | 87% | 24% | 49% | 44% | 166% | 196% | 39% | 34% | 53% | 55% | 83% | 91% |
| DLK1 | 70% | 159% | 122% | 164% | 139% | 197% | 155% | 93% | 176% | 211% | 93% | 118% | 240% | 167% |
| (Pref-1) | | | | | | | | | | | | | | |
| E2F4 (p107) | 76% | 85% | 77% | 75% | 76% | 105% | 77% | 64% | 88% | 84% | 77% | 82% | 90% | 82% |
| EIF4EBP1 | 97% | 75% | 75% | 72% | 86% | 99% | 76% | 84% | 77% | 97% | 51% | 96% | 109% | 68% |
| ELOVL3 | 180% | 117% | 79% | 71% | 172% | 82% | 50% | 62% | 58% | 113% | 134% | 92% | 116% | 117% |
| ESRRA | 118% | 100% | 74% | 84% | 106% | 94% | 56% | 54% | 74% | 75% | 71% | 67% | 98% | 62% |
| (NR3B1) | | | | | | | | | | | | | | |
| FGF16 | 74% | 62% | 41% | 70% | 32% | 55% | 40% | 60% | 36% | 101% | 43% | 56% | 32% | 61% |
| FGF19 | 77% | 137% | 136% | 194% | 174% | 170% | 70% | 147% | 75% | 179% | 36% | 118% | 195% | 155% |
| FGF21 | 61% | 121% | 175% | 189% | 233% | 132% | 145% | 130% | 118% | 693% | 176% | 177% | 358% | 345% |
| FNDC5 | 99% | 46% | 43% | 190% | 41% | 77% | 40% | 41% | 77% | 78% | 61% | 49% | 34% | 65% |
| FOXC2 | 87% | 299% | 136% | 133% | 89% | 179% | 455% | 389% | 183% | 113% | 109% | 135% | 132% | 83% |
| GDF3 | 94% | 42% | 58% | 49% | 63% | 47% | 58% | 67% | 40% | 55% | 55% | 53% | 59% | 54% |
| HCRT | 120% | 267% | 230% | 358% | 185% | 258% | 200% | 201% | 193% | 480% | 347% | 141% | 154% | 203% |
| (OREXIN) | | | | | | | | | | | | | | |
| HOXC8 | 69% | 81% | 105% | 97% | 94% | 97% | 128% | 126% | 115% | 108% | 95% | 112% | 75% | 132% |
| IKBKE | 81% | 93% | 102% | 119% | 104% | 132% | 68% | 96% | 123% | 85% | 119% | 78% | 88% | 78% |
| INSR | 93% | 96% | 80% | 74% | 96% | 103% | 51% | 52% | 84% | 105% | 76% | 75% | 95% | 65% |
| IRS1 | 70% | 94% | 109% | 111% | 124% | 109% | 79% | 79% | 97% | 109% | 90% | 96% | 101% | 118% |
| KDM3A | 87% | 85% | 97% | 115% | 90% | 131% | 104% | 129% | 105% | 110% | 89% | 85% | 72% | 107% |
| (JMJD1A) | | | | | | | | | | | | | | |
| KLF11 | 169% | 100% | 81% | 98% | 67% | 126% | 164% | 143% | 88% | 68% | 81% | 78% | 63% | 73% |
| KLF15 | 126% | 141% | 94% | 106% | 142% | 129% | 78% | 69% | 81% | 87% | 102% | 74% | 129% | 72% |
| KLF5 | 51% | 72% | 136% | 57% | 72% | 63% | 231% | 270% | 102% | 105% | 45% | 96% | 50% | 107% |
| LRP6 | 74% | 107% | 94% | 128% | 116% | 111% | 77% | 67% | 116% | 100% | 81% | 76% | 76% | 86% |
| MAPK14 | 90% | 107% | 109% | 105% | 117% | 140% | 86% | 89% | 104% | 134% | 105% | 129% | 144% | 108% |
| MED13 | 97% | 92% | 91% | 83% | 104% | 107% | 88% | 98% | 98% | 116% | 85% | 87% | 98% | 102% |
| NCOA1 | 89% | 114% | 123% | 134% | 145% | 143% | 97% | 107% | 145% | 146% | 109% | 120% | 128% | 116% |
| NCOA2 | 93% | 121% | 125% | 132% | 129% | 141% | 100% | 99% | 117% | 155% | 116% | 121% | 119% | 26% |
| NCOA3 | 77% | 102% | 95% | 86% | 82% | 94% | 123% | 121% | 92% | 76% | 72% | 107% | 70% | 119% |
| NR0B2 | 74% | 51% | 14% | 33% | 40% | 38% | 29% | 19% | 32% | 23% | 26% | 22% | 87% | 15% |
| (SHP) | | | | | | | | | | | | | | |
| NR1H3 | 292% | 69% | 90% | 122% | 105% | 125% | 37% | 40% | 96% | 90% | 97% | 112% | 118% | 90% |
| (LXRA) | | | | | | | | | | | | | | |
| NR4A3 | 39% | 135% | 79% | 66% | 47% | 89% | 25% | 28% | 53% | 74% | 49% | 85% | 81% | 65% |
| NRF1 | 76% | 77% | 95% | 94% | 111% | 106% | 63% | 71% | 95% | 79% | 83% | 70% | 81% | 81% |
| NRIP1 | 159% | 88% | 109% | 106% | 100% | 150% | 81% | 76% | 143% | 170% | 101% | 139% | 170% | 112% |
| (RIP140) | | | | | | | | | | | | | | |
| PLAC8 | 71% | 72% | 79% | 41% | 69% | 61% | 66% | 81% | 52% | 127% | 117% | 87% | 63% | 42% |
| PPARA | 58% | 130% | 113% | 82% | 144% | 113% | 71% | 69% | 90% | 118% | 99% | 96% | 124% | 87% |
| PPARD | 82% | 84% | 98% | 79% | 97% | 90% | 99% | 85% | 96% | 83% | 91% | 85% | 93% | 92% |
| PPARG | 70% | 110% | 104% | 111% | 144% | 125% | 64% | 58% | 97% | 122% | 110% | 104% | 129% | 102% |
| PPARGC1A | 93% | 68% | 72% | 76% | 84% | 108% | 36% | 41% | 75% | 90% | 66% | 79% | 90% | 83% |
| PPARGC1B | 120% | 64% | 62% | 65% | 106% | 96% | 39% | 39% | 79% | 125% | 64% | 87% | 117% | 80% |
| PRDM16 | 53% | 31% | 37% | 47% | 49% | 23% | 30% | 32% | 26% | 80% | 47% | 32% | 38% | 42% |
| PRDX3 | 114% | 95% | 108% | 94% | 148% | 102% | 66% | 68% | 94% | 120% | 109% | 126% | 136% | 122% |
| PRKAA1 | 75% | 87% | 113% | 91% | 118% | 96% | 99% | 97% | 81% | 84% | 76% | 88% | 90% | 100% |
| (AMPKA1) | | | | | | | | | | | | | | |
| PRKAA2 | 152% | 71% | 111% | 123% | 97% | 107% | 93% | 79% | 144% | 133% | 131% | 103% | 76% | 119% |
| (AMPKA2) | | | | | | | | | | | | | | |
| PRKACA | 155% | 95% | 103% | 102% | 113% | 135% | 69% | 73% | 126% | 132% | 93% | 123% | 132% | 101% |
| PRKACB | 106% | 63% | 97% | 103% | 104% | 101% | 113% | 104% | 83% | 118% | 106% | 97% | 97% | 109% |
| PRKAR1A | 83% | 100% | 98% | 123% | 107% | 104% | 95% | 97% | 100% | 103% | 100% | 71% | 73% | 104% |
| RB1 (pRb) | 91% | 76% | 95% | 88% | 90% | 90% | 89% | 89% | 103% | 69% | 81% | 95% | 94% | 89% |
| RPS6KB1 | 98% | 94% | 96% | 100% | 113% | 116% | 80% | 83% | 104% | 110% | 82% | 103% | 101% | 100% |
| RUNX1T1 | 57% | 114% | 98% | 117% | 91% | 137% | 95% | 101% | 115% | 73% | 89% | 71% | 60% | 90% |
| RUNX2 | 50% | 69% | 97% | 74% | 64% | 75% | 180% | 156% | 125% | 51% | 81% | 56% | 46% | 75% |
| SIRT1 | 97% | 108% | 114% | 112% | 119% | 128% | 98% | 98% | 108% | 112% | 75% | 110% | 120% | 112% |
| SIRT3 | 120% | 83% | 88% | 100% | 97% | 125% | 64% | 57% | 90% | 116% | 80% | 100% | 105% | 99% |
| SLC27A2 | 86% | 88% | 97% | 254% | 144% | 97% | 104% | 58% | 85% | 251% | 195% | 128% | 84% | 163% |
| (FATP2) | | | | | | | | | | | | | | |
| SREBF1 | 72% | 109% | 101% | 122% | 118% | 109% | 66% | 64% | 111% | 97% | 117% | 98% | 107% | 89% |
| SREBF2 | 86% | 122% | 91% | 121% | 92% | 119% | 95% | 105% | 124% | 72% | 123% | 103% | 95% | 87% |
| STAT5A | 132% | 95% | 105% | 101% | 95% | 122% | 53% | 56% | 82% | 131% | 88% | 115% | 152% | 91% |
| TNFRSF1A | 85% | 108% | 85% | 120% | 98% | 129% | 84% | 81% | 109% | 80% | 105% | 67% | 67% | 87% |
| TRPM8 | 464% | 83% | 106% | 145% | 203% | 124% | 162% | 73% | 155% | 305% | 208% | 143% | 244% | 141% |
| TRPV4 | 83% | 58% | 84% | 60% | 117% | 112% | 88% | 85% | 145% | 114% | 86% | 100% | 46% | 113% |
| TWIST1 | 54% | 120% | 96% | 121% | 91% | 109% | 143% | 110% | 99% | 88% | 102% | 61% | 131% | 68% |
| UCP1 | 749% | 268% | 237% | 292% | 466% | 212% | 313% | 246% | 210% | 369% | 221% | 263% | 604% | 223% |
| (SLC25A7) | | | | | | | | | | | | | | |
| UCP2 | 260% | 102% | 112% | 104% | 137% | 143% | 73% | 70% | 136% | 173% | 127% | 140% | 183% | 110% |
| (SLC25A8) | | | | | | | | | | | | | | |
| UCP3 | 78% | 146% | 117% | 132% | 135% | 129% | 135% | 105% | 117% | 269% | 62% | 125% | 123% | 205% |
| (SLC25A9) | | | | | | | | | | | | | | |
| WNT10B | 82% | 43% | 61% | 89% | 38% | 24% | 43% | 45% | 66% | 77% | 61% | 45% | 90% | 61% |
| WNT5A | 57% | 85% | 80% | 57% | 199% | 40% | 261% | 215% | 53% | 60% | 33% | 72% | 70% | 86% |

miRNA analogs with ≧2 fold increase of UCP1 gene (n = 13)
RSGLTZ; Rosiglitazone

### Example 5

### Development and Characterization of Clonal Nucleotide Aptamers Specifically Targeting Human Adipocytes or Related Cells

This example demonstrates how to generate an aptamer for use in an aptamir or an exomir. The Cell-SELEX technology was used to develop and characterize DNA or RNA aptamers that specifically recognize mature human subcutaneous adipocytes. SELEX may also be used.

### Pool Preparation

A starting pool of random ribonucleic acid sequences (30-50 nucleotides) is synthesized. Then, fixed sequences are added to the 5′ and 3′ of the 30-50 random nucleotides for efficient amplification. The pool is then amplified with the primers, and then used as a template for in vitro transcription with a modified T7 RNA polymerase. Transcriptions are typically incubated at 37° C. overnight, using 40 mM Tris pH 8.0, 40 mM DTT, 1 mM spermidine-HCl, 0.002% TritonX-100, 4% (w/v) PEG-8000, 12 mM MgCl₂, 3 mM 2′-F-CTP, 3 mM 2′-F-UTP, 3 mM GTP, 3 mM ATP, 0.5× inorganic pyrophosphatase, and 1× modified T7 polymerase, and approximately 0.5 M template RNA.

### Selection

Twenty rounds of cell-SELEX are initiated using both positive and negative selections. First, the RNA pools are incubated with non-target cells containing any one or more of the negative markers discussed above. RNA sequences that bind to the non-target cells are removed from the pool. Then, the remaining RNA sequences in the pool are incubated with target tissue or cells under conditions that are favorable for binding. Next, the unbound RNA sequences are partitioned from those RNA sequences that bound to the target tissue or cells. Then, the bound sequences are dissociated from the target tissue or cells, and reverse transcribed. The resulting cDNA is used as a template for PCR using primers and Taq polymerase. PCR reactions are done under the following conditions: a) denaturation step: 94° C. for 2 minutes; b) cycling steps: 94° C. for 30 seconds, 60° C. for 30 seconds, 72° C. for 1 minute; c) final extension step: 72° C. for 3 minutes. The cycles are repeated until sufficient PCR product is generated. The amplified pool template DNA is then isopropanol precipitated and half of the PCR product is used as template for the transcription of pool RNA for the next round of selection. The transcribed RNA pool is gel purified using a 10% polyacrylamide gel every third round. When not gel-purified, the transcribed RNA pool is desalted. In all cases, an equivalent of one-tenth of the total transcription product is carried forward as the starting pool for the subsequent round of selection.

### Aptamer Generation

After the Cell-SELEX process, approximately 10-100 nucleic acid sequences are chosen for further screening and evaluation. Aptamers with the desired binding affinity (K_{d}) and specificity are isolated from the 10-100 nucleic acid sequences. Typically, 1-4 sequences are chosen for further examination. Each of these 1-4 sequences is then minimized to determine the smallest, most stable aptamer sequence that will bind to the target tissue or cells.

Once the minimized sequence is determined, the aptamer is modified. For example, the aptamer may be capped, such as with a 3′ inverted nucleotide (e.g., dT) cap; 2′-F and/or 2′-OMe moieties may be added to the aptamer; phosphorothioate linkages may be added to the aptamer; and a PEG molecule may be added to the aptamer.

As an example of Cell-SELEX, negative selection was performed with freshly isolated human hepatocytes and positive selection utilized primary cultures of human subcutaneous adipocytes. Two rounds of negative selection and five rounds of positive selection from a 32-mer library were completed. Isolated aptamers were sequenced, synthesized and labeled with 6-fluorescein amidite (FAM) for binding studies.

### In Vitro Validation of the Aptamers Identified by Cell-SELEX

The extent and kinetic profile of fluorescently-tagged aptamers binding to intact target cells is performed by FACS in cultured ATMSCs, WAT and BAT (and negative cell lines, e.g. HepG2 cells). Cells are counterstained with 4′,6-diamidino-2-phenylindole (DAPI) to assess cellular distribution (cytoplasmic vs. nuclear) of aptamer binding. Surface fluorescence is measured by flow cytometry. Cellular uptake and localization are studied by non-confocal microscopy. Cell viability is assessed with MTT.

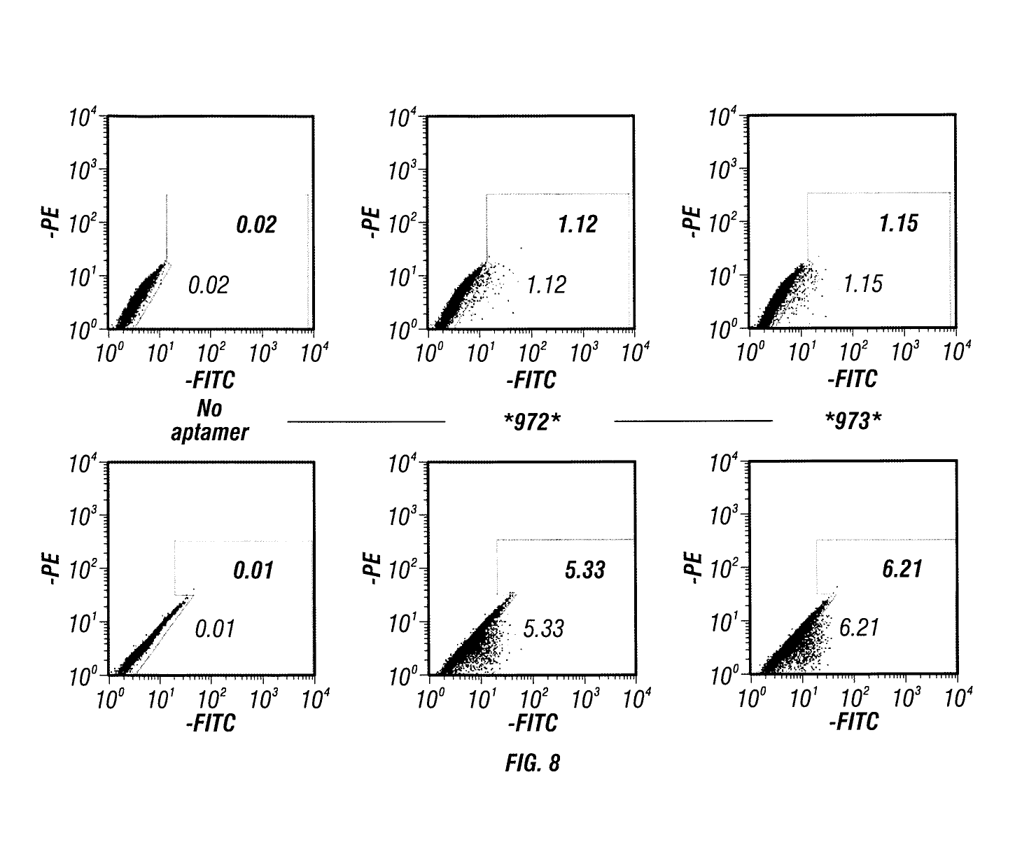

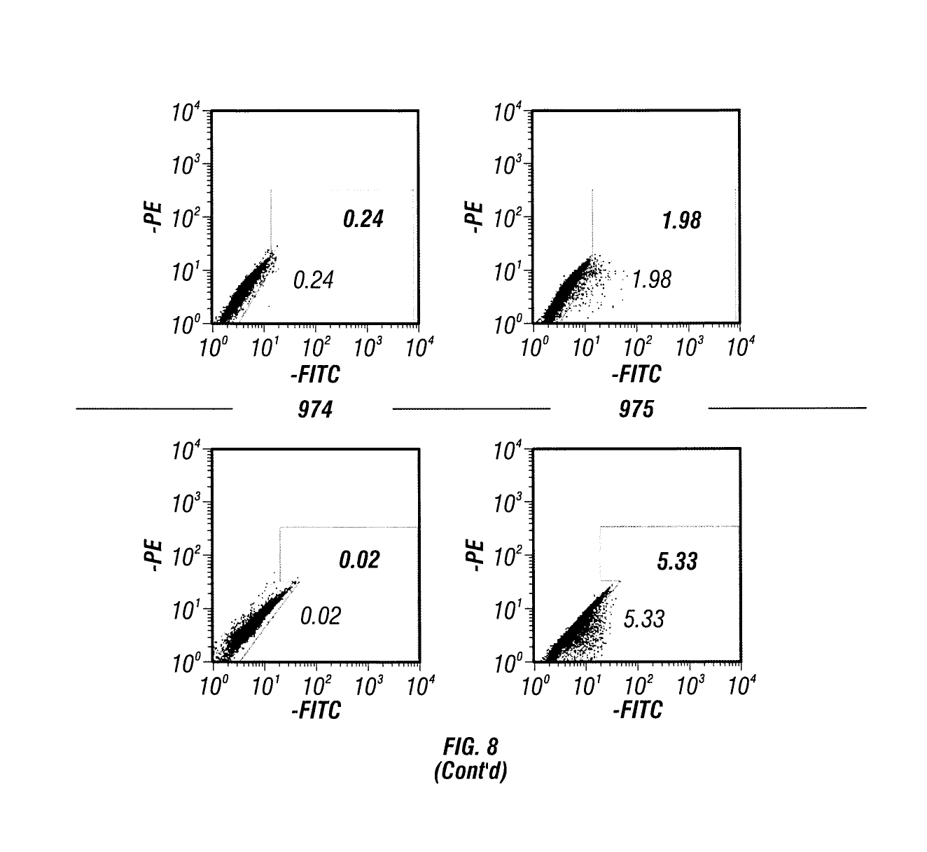

In the example above, human hepatocytes (negative cells) and adipocytes (positive cells) were labeled for 15 minutes at room temperature with a saturating concentration (1 μM) of 6-fluorescein amidite (FAM)-conjugated aptamers and analyzed by fluorescence-activated cell sorting (FACS). As shown on FIG. 8, some aptamers (e.g., aptamer 974) do not bind to adipocytes nor hepatocytes, some aptamers (e.g., aptamer 975 bind to both adipocytes and hepatocytes, ratio: 2.69) and other aptamers bind preferentially to adipocytes (e.g., aptamers 972 and 973, ratio: 4.76 and 5.40, respectively). Further characterization of these adipocyte-specific aptamers is in progress through additional rounds of negative and positive selection.

### Example 6

### Development of Customized Exosomes

This example demonstrates how exosome vesicles that can be customized to specifically deliver their load of miRNA modulators to targeted adipocytes and enhance their intra-cellular penetration while protecting them from degradation.

For the purpose of creating de novo exosomes that can safely and efficiently carry and deliver their miRNA analogs load to adipocytes and related cells, the following criteria are employed (using ExoCarta 2012, a repository database of exosomal proteins, mRNAs, miRNAs and lipids (available online at exocarta.org), Vesiclepedia, a manually curated compendium of molecular data of extracellular vesicles (available online at microvesicles.org) and EVpedia, an integrated and comprehensive proteome, transcriptome, and lipidome database of extracellular vesicles (available online at evpedia.info), as references): (1) Elimination of antigen-presenting molecules to avoid the risk of triggering an innate immune reaction; (2) Restriction of the exosome size to the 30 to 100 nm range, also to avoid the risk of triggering an innate immune reaction and to reduce clearance by the mononuclear phagocyte system; (3) Addition of lipids, lipid rafts and cytoskeleton proteins; (4) Addition of transmembrane proteins like tetraspanins, LAMPs, CD13 and PGRL; (5) Addition of membrane transport and fusion proteins like caveolins; (6) Addition of adipocyte-specific glycosylphosphatidylinositol (GPI)-anchored proteins like CD73; (7) Addition of molecules that are preferentially expressed at the surface of human adipocytes (see Table 7).

TABLE 7

| Name | Entrez Gene ID | Ensembl Gene ID |
|---|---|---|
| DPT (Dermatopontin) | 1805 | ENSG00000143196 |
| CD10 (MME) | 4311 | ENSG00000196549 |
| FABP4 | 2167 | ENSG00000170323 |
| CD36 (FAT) | 948 | ENSG00000135218 |
| Caveolin 1 (CAV1) | 857 | ENSG00000105974 |
| CD91 (LRP1) | 4035 | ENSG00000123384 |
| Stomatin | 2040 | ENSG00000148175 |
| Mfge8 (Lactadherin) | 4240 | ENSG00000140545 |
| ITGA7 (integrin, alpha 7) | 3679 | ENSG00000135424 |
| Caveolin 2 (CAV2) | 858 | ENSG00000105971 |
| ANXA5 (Annexin 5) | 308 | ENSG00000164111 |
| CD90 (THY1) | 7070 | ENSG00000154096 |
| ANXA2 (Annexin 2) | 302 | ENSG00000182718 |
| ANXA1 (Annexin 1) | 301 | ENSG00000135046 |
| CD146 (MCAM) | 4162 | ENSG00000076706 |
| CD151 (TSPAN24) | 977 | ENSG00000177697 |
| CD29 (ITGB1) | 3688 | ENSG00000150093 |
| CD166 (ALCAM) | 214 | ENSG00000170017 |
| Lamp1 | 3916 | ENSG00000185896 |
| CD13 (ANPEP) | 290 | ENSG00000166825 |
| CD63 | 967 | ENSG00000135404 |
| ANXA6 (Annexin 6) | 309 | ENSG00000197043 |
| GYPC (Glycophorin C) | 2995 | ENSG00000136732 |
| CD49e (ITGA4) | 3678 | ENSG00000161638 |
| TSPAN3 | 10099 | ENSG00000140391 |
| CD81 (TSPAN28) | 975 | ENSG00000110651 |
| TSPAN4 | 7106 | ENSG00000214063 |
| ANXA4 (Annexin 4) | 307 | ENSG00000196975 |
| SXT10 (Syntaxin 10) | 8677 | ENSG00000104915 |
| Alix (PDCD6IP) | 10015 | ENSG00000170248 |
| NPR1 | 4881 | ENSG00000169418 |
| ANXA7 (Annexin 7) | 310 | ENSG00000138279 |
| CD44 (Hyaluronate) | 960 | ENSG00000026508 |

### Example 6

### In Vitro Validation of the Thermogenic Aptamirs

Once the aptamer and miRNA elements are each validated in vitro, their combinations (the aptamirs and exomirs) are validated in vitro, using the same techniques as described above.

### Example 7

### In Vivo Validation of the Thermogenic Aptamirs in Animal Models of Obesity

Several animal models of obesity have been developed and validated (Kanasaki, et al., 2011; Speakman, et al., 2007). The most commonly used are the Leptin Signaling Defects Lepob/ob and Leprdb/db Mouse Models as well as the High-Fat Diet model in C57BL/6J mice (Wang, et al., 2012). This diet-induced obesity (DIO) model closely mimics the increased availability of the high-fat/high-density foods in modern society.

C57Bl/6 mice fed a high fat diet are used to assess the efficacy and safety of the thermogenic aptamirs, following the protocol described by Esau, et al., 2006, exploring the effects of miR-122 inhibition on lipid metabolism. Thus, a DIO mouse model is used for in vivo validation of the effectiveness of the miRNA analogs described herein for the increase in thermogenesis and/or the treatment of obesity and other metabolic disorders (Yin, et al., 2013). DIO mice are administered one or more of an agomir, antagomir, aptamir or exomir. Rosiglitazone is used as a positive control. Food intake, blood metabolic parameters, body composition (body weight, body fat, bone mineral and lean mass, body fat distribution, body temperature, 02 consumption and C02 production, exercise induced thermogenesis, cold induced thermogenesis and resting thermogenesis are measured in the mice prior to and after treatment. A reduction in body mass or body fat or an increase in body temperature or any kind of thermogenesis indicate the in vivo effectiveness of the administered composition.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ausubel et al., Current Protocols in Molecular Biology, John Wiley & Sons, Inc., 1994.
Bader, et al., Gene Ther. 18(12):1121-1126, 2011.
Bobrie, et al., Traffic. 12:1665-1668, 2011
Braasch & Corey, Biochemistry. 41(14):4503-4510, 2002.
Broderick, et al., Gene Ther. 18(12):1104-1110, 2011.
Brophy, Eur. J. Clin. Pharmacol. 24:103-108, 1983.
Crooke et al., J. Pharmacol. Exp. Ther. 277:923-937, 1996.
Czech, et al., Nat. Rev. Endocrinol. 7(8):473-484, 2011.
De Mesmaeker, et al. Ace. Chem. Res. 28:366-374, 1995
Elmen, et al., Nature. 452:896-899, 2008.
Esau et al, Cell Metab. 3(2):87-98, 2006.
Eyles, J. Pharm. Pharmacol. 49:669-674, 1997.
Fotherby, Contraception. 54:59-69, 1996.
Freshney, Animal Cell Culture, 1986.
Fujiki, et al. BMC Biol. 2009.
Gait, Oligonucleotide Synthesis, 1984.
Gallagher, et al. Genome medicine. 2010.
Gao, Pharm. Res. 12:857-863, 1995.
Gebeyehu, et al. Nucl. Acids Res. 15:4513, 1987.
Genesis, 30:3, 2001.
Glover, DNA Cloning: A Practical Approach, Volumes I and II, 1985.
Groning, Pharmazie. 51:337-341, 1996.
Hames & Higgins, Nucleic Acid Hybridization, 1985.
Hames & Higgins, Transcription And Translation, 1984.
Hao, et al., Mol Biosyst. 2012.
Heasman, J., Dev. Biol., 2002, 243, 209-214, 2002
Hidalgo-Aragones, J. Steroid Biochem. Mol. Biol. 58:611-617, 1996.
Immobilized Cells And Enzymes, IRL Press, 1986.
International Application Serial No. PCT/US2013/037579
Johnson, J. Pharm. Sci. 84:1144-1146, 1995.
Johnstone, et al., J Biol Chem. 262:9412-9420, 1987
Kabanov et al., FEBS Lett. 259:327-330, 1990.
Kanasaki et al., J. Biomed. Biotechnol. 2011:197636, 2011.
Kornberg, DNA Replication, W. H. Freeman & Co.: San Francisco, pp. 75-77, 1980.
Krutzfeldt, et al., Nature. 438:685-689, 2005.
Lacerra et al., Proc. Natl. Acad. Sci. 97:9591-9596, 2000.
Lee, et al., Semin Immunopathol. 33:455-467, 2011.
Lennox, et al., Gene Ther. 18(12):1111-1120, 2011.
Letsinger et al., Proc. Natl. Acad. Sci. USA. 86:6553-6556, 1989.
Lindstrom, Scientific World Journal, 7:666-685, 2007.
Mancharan et al., Nucleosides & Nucleotides. 14:969-973, 1995a.
Manoharan et al., Ann. N. Y. Acad. Sci. 660:306-309, 1992.
Manoharan et al., Bioorg. Med. Chem. Let. 4:1053-1060, 1994.
Manoharan et al., Bioorg. Med. Chem. Let., 3:2765-2770, 1993.
Manoharan et al., Tetrahedron Lett. 36:3651-3654, 1995b.
Minto, J. Pharmacol. Exp. Ther. 281:93-102, 1997.
Mishra et al., Biochim. Biophys. Acta. 1264:229-237, 1995.
Müller, Cellular Signalling. 23:1207-1223, 2011
Nasevicius et al., Nat. Genet., 26: 216-220, 2000.
Nielsen, et al., Science. 254:1497, 1991.
Oberhauser et al., Nucl. Acids Res. 20:533-538, 1992.
Okada, et al., J Androl. 2010.
Perbal, A Practical Guide To Molecular Cloning, 1984.
Rao, J. Biomater Sci. Polym. Ed. 7:623-645, 1995.
Raposo, et al., J Exp Med. 183:1161-1172, 1996.
Remington: The Science and Practice of Pharmacy, 21st ed., 2005
Rohatagi, J. Clin. Pharmacol. 35: 1 187-1193, 1995.
Rohatagi, Pharmazie 50:610-613, 1995.
Sambrook, et al., Molecular Cloning: A Laboratory Manual, 2^{nd} Edition, Cold Spring Harbor Laboratory Press: New York, 1989.
Sanghvi, et al., Antisense Research & Applications, CRC Press: Boca Raton, pp. 276-278, 1993.
Schorey & Bhatnagar, Traffic. 9:871-881, 2008.
Sharma, et al., Am J Physiol Renal Physiol., 284(6):F1138-1144, 2003.
Shea et al., Nucl. Acids Res. 18:3777-3783, 1990
Skog, et al., Nat Cell Biol. 10:1470-1476, 2008
Snead, et al., Nucleic Acid Ther. 22(3):139-146, 2012.
Speakman et al., Obesity reviews. 8(1):55-61, 2007.
Svinarchuk et al., Biochimie. 75:49-54, 1993.
Thorsen, et al., Cancer J. 18(3):275-284, 2012.
Tjwa, Ann. Allergy Asthma Immunol. 75: 107-111, 1995.
U.S. application Ser. No. 13/826,775
U.S. Patent Application No. 61/636,059
U.S. Pat. No. 3,687,808
U.S. Pat. No. 4,469,863
U.S. Pat. No. 4,476,301
U.S. Pat. No. 4,587,044
U.S. Pat. No. 4,605,735
U.S. Pat. No. 4,667,025
U.S. Pat. No. 4,762,779
U.S. Pat. No. 4,789,737
U.S. Pat. No. 4,824,941
U.S. Pat. No. 4,828,979
U.S. Pat. No. 4,835,263
U.S. Pat. No. 4,845,205
U.S. Pat. No. 4,876,335
U.S. Pat. No. 4,904,582
U.S. Pat. No. 4,948,882
U.S. Pat. No. 4,958,013
U.S. Pat. No. 5,013,830
U.S. Pat. No. 5,023,243
U.S. Pat. No. 5,034,506
U.S. Pat. No. 5,082,830
U.S. Pat. No. 5,109,124
U.S. Pat. No. 5,112,963
U.S. Pat. No. 5,118,802
U.S. Pat. No. 5,130,302
U.S. Pat. No. 5,134,066
U.S. Pat. No. 5,138,045
U.S. Pat. No. 5,149,797
U.S. Pat. No. 5,166,315
U.S. Pat. No. 5,175,273
U.S. Pat. No. 5,177,196
U.S. Pat. No. 5,185,444
U.S. Pat. No. 5,188,897
U.S. Pat. No. 5,214,134
U.S. Pat. No. 5,214,136
U.S. Pat. No. 5,216,141
U.S. Pat. No. 5,218,105
U.S. Pat. No. 5,220,007
U.S. Pat. No. 5,235,033
U.S. Pat. No. 5,245,022
U.S. Pat. No. 5,254,469
U.S. Pat. No. 5,256,775
U.S. Pat. No. 5,258,506
U.S. Pat. No. 5,262,536
U.S. Pat. No. 5,264,423
U.S. Pat. No. 5,264,562
U.S. Pat. No. 5,264,564
U.S. Pat. No. 5,270,163
U.S. Pat. No. 5,272,250
U.S. Pat. No. 5,276,019
U.S. Pat. No. 5,278,302
U.S. Pat. No. 5,286,717
U.S. Pat. No. 5,292,873
U.S. Pat. No. 5,317,098
U.S. Pat. No. 5,321,131
U.S. Pat. No. 5,366,878
U.S. Pat. No. 5,367,066
U.S. Pat. No. 5,371,241
U.S. Pat. No. 5,391,723
U.S. Pat. No. 5,399,676
U.S. Pat. No. 5,403,711
U.S. Pat. No. 5,405,938
U.S. Pat. No. 5,405,939
U.S. Pat. No. 5,414,077
U.S. Pat. No. 5,416,203
U.S. Pat. No. 5,432,272
U.S. Pat. No. 5,434,257
U.S. Pat. No. 5,451,463
U.S. Pat. No. 5,453,496
U.S. Pat. No. 5,455,233
U.S. Pat. No. 5,457,187
U.S. Pat. No. 5,459,255
U.S. Pat. No. 5,466,677
U.S. Pat. No. 5,470,967
U.S. Pat. No. 5,475,096
U.S. Pat. No. 5,476,925
U.S. Pat. No. 5,484,908
U.S. Pat. No. 5,486,603
U.S. Pat. No. 5,489,677
U.S. Pat. No. 5,491,133
U.S. Pat. No. 5,502,177
U.S. Pat. No. 5,510,475
U.S. Pat. No. 5,512,439
U.S. Pat. No. 5,512,667
U.S. Pat. No. 5,514,785
U.S. Pat. No. 5,519,126
U.S. Pat. No. 5,525,465
U.S. Pat. No. 5,525,711
U.S. Pat. No. 5,536,821
U.S. Pat. No. 5,539,082
U.S. Pat. No. 5,541,306
U.S. Pat. No. 5,541,307
U.S. Pat. No. 5,541,313
U.S. Pat. No. 5,545,730
U.S. Pat. No. 5,550,111
U.S. Pat. No. 5,552,538
U.S. Pat. No. 5,552,540
U.S. Pat. No. 5,561,225
U.S. Pat. No. 5,563,253
U.S. Pat. No. 5,565,350
U.S. Pat. No. 5,565,552
U.S. Pat. No. 5,567,810
U.S. Pat. No. 5,571,799
U.S. Pat. No. 5,574,142
U.S. Pat. No. 5,578,717
U.S. Pat. No. 5,578,718
U.S. Pat. No. 5,580,731
U.S. Pat. No. 5,582,981
U.S. Pat. No. 5,585,481;
U.S. Pat. No. 5,587,361
U.S. Pat. No. 5,587,371
U.S. Pat. No. 5,587,469
U.S. Pat. No. 5,591,584
U.S. Pat. No. 5,595,726
U.S. Pat. No. 5,595,877
U.S. Pat. No. 5,596,086
U.S. Pat. No. 5,596,091
U.S. Pat. No. 5,597,696
U.S. Pat. No. 5,599,928
U.S. Pat. No. 5,599,923
U.S. Pat. No. 5,602,240
U.S. Pat. No. 5,608,046
U.S. Pat. No. 5,610,289
U.S. Pat. No. 5,614,617
U.S. Pat. No. 5,618,704
U.S. Pat. No. 5,623,065
U.S. Pat. No. 5,623,070
U.S. Pat. No. 5,625,050
U.S. Pat. No. 5,633,360
U.S. Pat. No. 5,637,459
U.S. Pat. No. 5,652,355
U.S. Pat. No. 5,652,356
U.S. Pat. No. 5,663,312
U.S. Pat. No. 5,677,437
U.S. Pat. No. 5,677,439
U.S. Pat. No. 5,681,941
U.S. Pat. No. 5,688,941
U.S. Pat. No. 5,700,922
U.S. Pat. No. 5,714,331
U.S. Pat. No. 5,716,928
U.S. Pat. No. 5,719,262
U.S. Pat. No. 5,750,692
U.S. Pat. No. 5,858,401
U.S. Pat. No. 6,107,094
U.S. Patent Publication No. 2004/0028670
van Rooij, et al., Circ. Res., 110(3):496-507, 2012.
Wang et al., J. Am. Chem. Soc. 122:8595-8602, 2000.
Wang et al., Methods in molecular biology. 821:421-433, 2012.
Yin et al., Cell Metab. 17(2):210-224, 2013.
Zhou, Intern. J. Biol. Sci. 8(1):118-123, 2012

1. A composition comprising a miRNA agent and a targeting agent.

2. The composition of claim 1, wherein the aptamir composition is an adipocyte-specific aptamir composition.

3. The composition of claim 1, wherein the miRNA agent is a miRNA, a miRNA agomir, or a miRNA antagomir.

4. The composition of claim 1, wherein the miRNA agent is thermogenic, adipogenic, or thermogenic and adipogenic.

5.-8. (canceled)

9. The composition of claim 3, wherein the miRNA agent is at least 75% identical, preferably 80% identical, more preferably 85% identical, more preferably 90% identical, more preferably 95% identical, more preferably 100% identical to the sequence of a miRNA selected from the group consisting of hsa-miR-515-3p, hsa-miR-141-3p, hsa-miR-20a-5p, hsa-miR-16-5p, hsa-miR-125a-5p, hsa-miR-let-7d-3p, hsa-miR-371a-3p, hsa-miR-195-5p, hsa-miR-106b-3p, hsa-miR-605, hsa-miR-217, hsa-miR-148a-3p, hsa-miR-19b-3p, hsa-miR-192-5p, hsa-miR-101-5p, hsa-miR-200b-3p, hsa-miR-487b, hsa-miR-1179, hsa-miR-223-3p, hsa-let-7a-3p, hsa-let-7a-5p, hsa-let-7b-3p, hsa-let-7b-5p, hsa-let-7c, hsa-let-7d-5p, hsa-let-7e-3p, hsa-let-7c-5p, hsa-let-7f-1-3p, hsa-let-7f-2-3p, hsa-let-7f-5p, hsa-let-7g-3p, hsa-let-7g-5p, hsa-let-7i-3p, hsa-let-7i-5p, hsa-miR-1, hsa-miR-100-3p, hsa-miR-100-5p, hsa-miR-101-3p, hsa-miR-103a-2-5p, hsa-miR-103a-3p, hsa-miR-103b, hsa-miR-105-5p, hsa-miR-106a-3p, hsa-miR-106a-5p, hsa-miR-106b-5p, hsa-miR-107, hsa-miR-10a-3p, hsa-miR-10a-5p, hsa-miR-0b-3p, hsa-miR-10b-5p, hsa-miR-1178-3p, hsa-miR-1180, hsa-miR-1181, hsa-miR-1182, hsa-miR-1183, hsa-miR-1184, hsa-miR-1185-5p, hsa-miR-1204, hsa-miR-1207, hsa-miR-1208, hsa-miR-122-3p, hsa-miR-122-5p, hsa-miR-1224, hsa-miR-1226, hsa-miR-1227-3p, hsa-miR-1228-5p, hsa-miR-1229-3p, hsa-miR-1231, hsa-miR-124-3p, hsa-miR-1245a, hsa-miR-1246, hsa-miR-1249, hsa-miR-125a-3p, hsa-miR-125b-1-3p, hsa-miR-125b-2-3p, hsa-miR-125b-5p, hsa-miR-1251, hsa-miR-1252, hsa-miR-1253, hsa-miR-1255a, hsa-miR-1255b-5p, hsa-miR-126-3p, hsa-miR-1260a, hsa-miR-1260b, hsa-miR-1262, hsa-miR-1263, hsa-miR-1265, hsa-miR-1268a, hsa-miR-127-3p, hsa-miR-1270, hsa-miR-1272, hsa-miR-1273a, hsa-miR-1275, hsa-miR-1276, hsa-miR-1277-3p, hsa-miR-1278, hsa-miR-128, hsa-miR-1285-3p, hsa-miR-1286, hsa-miR-1287, hsa-miR-129-2-3p, hsa-miR-1292-5p, hsa-miR-1293, hsa-miR-1301, hsa-miR-1302, hsa-miR-1303, hsa-miR-1305, hsa-miR-130a-3p, hsa-miR-130a-5p, hsa-miR-130b-3p, hsa-miR-130b-5p, hsa-miR-132-3p, hsa-miR-132-5p, hsa-miR-1321, hsa-miR-1323, hsa-miR-1324, hsa-miR-133a, hsa-miR-133b, hsa-miR-134, hsa-miR-135a-3p, hsa-miR-135a-5p, hsa-miR-135b-3p, hsa-miR-135b-5p, hsa-miR-136-3p, hsa-miR-136-5p, hsa-miR-137, hsa-miR-138-1-3p, hsa-miR-138-2-3p, hsa-miR-138-5p, hsa-miR-139-3p, hsa-miR-139-5p, hsa-miR-140-3p, hsa-miR-140-5p, hsa-miR-142-3p, hsa-miR-142-5p, hsa-miR-143-3p, hsa-miR-143-5p, hsa-miR-144-3p, hsa-miR-144-5p, hsa-miR-145-3p, hsa-miR-145-5p, hsa-miR-1468, hsa-miR-146a-5p, hsa-miR-146b-3p, hsa-miR-146b-5p, hsa-miR-147a, hsa-miR-147b, hsa-miR-148a-5p, hsa-miR-148b-3p, hsa-miR-148b-5p, hsa-miR-149-3p, hsa-miR-149-5p, hsa-miR-150-3p, hsa-miR-150-5p, hsa-miR-151a-3p, hsa-miR-15a-5p, hsa-miR-152, hsa-miR-153, hsa-miR-1538, hsa-miR-1539, hsa-miR-154-3p, hsa-miR-154-5p, hsa-miR-155-5p, hsa-miR-15a-3p, hsa-miR-15a-5p, hsa-miR-15b-3p, hsa-miR-15b-5p, hsa-miR-16-1-3p, hsa-miR-16-2-3p, hsa-miR-17-3p, hsa-miR-17-5p, hsa-miR-181a-2-3p, hsa-miR-181a-3p, hsa-miR-181a-5p, hsa-miR-181 b-5p, hsa-miR-181c-3p, hsa-miR-181c-5p, hsa-miR-181d, hsa-miR-182-5p, hsa-miR-1827, hsa-miR-183-3p, hsa-miR-183-5p, hsa-miR-184, hsa-miR-185-3p, hsa-miR-185-5p, hsa-miR-186-3p, hsa-miR-186-5p, hsa-miR-187-3p, hsa-miR-187-5p, hsa-miR-188-5p, hsa-miR-18a-3p, hsa-miR-18a-5p, hsa-miR-18b-3p, hsa-miR-18b-5p, hsa-miR-190a, hsa-miR-190b, hsa-miR-191-3p, hsa-miR-191-5p, hsa-miR-1915-3p, hsa-miR-192-3p, hsa-miR-193a-3p, hsa-miR-193a-5p, hsa-miR-193b-3p, hsa-miR-193b-5p, hsa-miR-194-5p, hsa-miR-195-3p, hsa-miR-196a-3p, hsa-miR-196a-5p, hsa-miR-196b-5p, hsa-miR-197-3p, hsa-miR-198, hsa-miR-199a-3p, hsa-miR-199a-5p, hsa-miR-199b-3p, hsa-miR-199b-5p, hsa-miR-19a-3p, hsa-miR-19a-5p, hsa-miR-1909-3p, hsa-miR-1911-3p, hsa-miR-200a-3p, hsa-miR-200a-5p, hsa-miR-200b-5p, hsa-miR-200c-3p, hsa-miR-200c-5p, hsa-miR-202-3p, hsa-miR-202-5p, hsa-miR-203a, hsa-miR-203b-5p, hsa-miR-204-5p, hsa-miR-205-5p, hsa-miR-206, hsa-miR-208b, hsa-miR-20a-3p, hsa-miR-20b-3p, hsa-miR-20b-5p, hsa-miR-21-3p, hsa-miR-21-5p, hsa-miR-210, hsa-miR-211-5p, hsa-miR-2110, hsa-miR-212-3p, hsa-miR-214-3p, hsa-miR-214-5p, hsa-miR-215, hsa-miR-216a-5p, hsa-miR-218-1-3p, hsa-miR-218-5p, hsa-miR-219-1-3p, hsa-miR-219-5p, hsa-miR-22-3p, hsa-miR-22-5p, hsa-miR-221-3p, hsa-miR-221-5p, hsa-miR-222-3p, hsa-miR-222-5p, hsa-miR-223-5p, hsa-miR-224-3p, hsa-miR-224-5p, hsa-miR-2355-3p, hsa-miR-23a-3p, hsa-miR-23a-5p, hsa-miR-23b-3p, hsa-miR-23b-5p, hsa-miR-24-1-5p, hsa-miR-24-2-5p, hsa-miR-24-3p, hsa-miR-25-3p, hsa-miR-25-5p, hsa-miR-26a-2-3p, hsa-miR-26a-5p, hsa-miR-26b-3p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a-3p, hsa-miR-29a-5p, hsa-miR-29b-1-5p, hsa-miR-29b-2-5p, hsa-miR-29b-3p, hsa-miR-29c-3p, hsa-miR-29c-5p, hsa-miR-2909, hsa-miR-301a-3p, hsa-miR-301b, hsa-miR-302a-5p, hsa-miR-302b-5p, hsa-miR-302c-5p, hsa-miR-302d-3p, hsa-miR-302d-5p, hsa-miR-3021: hsa-miR-3064-5p, hsa-miR-3065-3p, hsa-miR-3065-5p, hsa-miR-3074-3p, hsa-miR-3074-5p, hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3120-3p, hsa-miR-3120-5p, hsa-miR-3184-5p, hsa-miR-32-3p, hsa-miR-32-5p, hsa-miR-320a, hsa-miR-320b, hsa-miR-320c, hsa-miR-323a-3p, hsa-miR-323b-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-5p, hsa-miR-346, hsa-miR-34a-3p, hsa-miR-34a-5p, hsa-miR-34b-3p, hsa-miR-34b-5p, hsa-miR-34c-3p, hsa-miR-34c-5p, hsa-miR-3591-3p, hsa-miR-361-3p, hsa-miR-361-5p, hsa-miR-3613-5p, hsa-miR-3615, hsa-miR-3619, hsa-miR-362-3p, hsa-miR-362-5p, hsa-miR-363-3p, hsa-miR-363-5p, hsa-mir-365a-3p, hsa-mir-365a-5p, hsa-mir-3653, hsa-miR-3656, hsa-miR-367-3p, hsa-miR-367-5p, hsa-miR-3676-3p, hsa-miR-369-3p, hsa-miR-369-5p, hsa-miR-370, hsa-miR-372, hsa-miR-373-3p, hsa-miR-373-5p, hsa-miR-374a-3p, hsa-miR-374a-5p, hsa-miR-374b-3p, hsa-miR-374b-5p, hsa-miR-375, hsa-miR-376a-2-5p, hsa-miR-376a-3p, hsa-miR-376a-5p, hsa-miR-376b-3p, hsa-miR-376c-3p, hsa-miR-377-3p, hsa-miR-377-5p, hsa-miR-378a-3p, hsa-miR-378a-5p, hsa-miR-378c, hsa-miR-378d, hsa-miR-379-3p, hsa-miR-379-5p, hsa-miR-380-3p, hsa-miR-380-5p, hsa-miR-381-3p, hsa-miR-382-3p, hsa-miR-382-5p, hsa-miR-383, hsa-miR-384, hsa-miR-3912, hsa-miR-3928, hsa-miR-409-3p, hsa-miR-409-5p, hsa-miR-410, hsa-miR-411-3p, hsa-miR-411-5p, hsa-miR-412, hsa-miR-421, hsa-miR-422a, hsa-miR-423-3p, hsa-miR-423-5p, hsa-miR-424-3p, hsa-miR-424-5p, hsa-miR-425-3p, hsa-miR-425-5p, hsa-miR-429, hsa-miR-4291, hsa-miR-431-5p, hsa-miR-432-5p, hsa-miR-433, hsa-miR-4421, hsa-miR-449a, hsa-miR-450a-5p, hsa-miR-450b-3p, hsa-miR-450b-5p, hsa-miR-4505, hsa-miR-4510, hsa-miR-4516, hsa-miR-451a, hsa-miR-452-3p, hsa-miR-452-5p, hsa-miR-4533, hsa-miR-4539, hsa-miR-454-3p, hsa-miR-454-5p, hsa-miR-455-3p, hsa-miR-455-5p, hsa-miR-4634, hsa-miR-4732-3p, hsa-miR-4732-5p, hsa-miR-4747-5p, hsa-miR-4792, hsa-miR-483-3p, hsa-miR-483-5p, hsa-miR-484, hsa-miR-485-5p, hsa-miR-486-3p, hsa-miR-486-5p, hsa-miR-489, hsa-miR-490, hsa-miR-491-3p, hsa-miR-491-5p, hsa-miR-492, hsa-miR-493-3p, hsa-miR-493-5p, hsa-miR-494, hsa-miR-495-3p, hsa-miR-496, hsa-miR-497-3p, hsa-miR-497-5p, hsa-miR-498, hsa-miR-499a-5p, hsa-miR-500a-3p, hsa-miR-501-3p, hsa-miR-501-5p, hsa-miR-502-3p, hsa-miR-502-5p, hsa-miR-503-5p, hsa-miR-504, hsa-miR-505-3p, hsa-miR-505-5p, hsa-miR-506-3p, hsa-miR-508-3p, hsa-miR-508-5p, hsa-miR-509-3p, hsa-miR-511, hsa-miR-512-5p, hsa-miR-513a-3p, hsa-miR-513a-5p, hsa-miR-513b, hsa-miR-514a-3p, hsa-miR-514a-5p, hsa-miR-515-5p, hsa-miR-516b-3p, hsa-miR-516b-5p, hsa-miR-517a-3p, hsa-miR-518a-3p, hsa-miR-518b, hsa-miR-518e-3p, hsa-miR-58e-5p, hsa-miR-518f-3p, hsa-miR-519a-5p, hsa-miR-519b-5p, hsa-miR-519c-3p, hsa-miR-519c-5p, hsa-miR-519d, hsa-miR-519e-5p, hsa-miR-520c-3p, hsa-miR-520e, hsa-miR-520f, hsa-miR-520g, hsa-miR-520h, hsa-miR-521, hsa-miR-522-5p, hsa-miR-523-5p, hsa-miR-525-3p, hsa-miR-532-3p, hsa-miR-532-5p, hsa-miR-539-5p, hsa-miR-541, hsa-miR-542-3p, hsa-miR-542-5p, hsa-miR-543, hsa-miR-545-3p, hsa-miR-545-5p, hsa-miR-548a-3p, hsa-miR-548d-3p, hsa-miR-548e, hsa-miR-548i, hsa-miR-548m, hsa-miR-549, hsa-miR-550a-3p, hsa-miR-550a-5p, hsa-miR-551b-3p, hsa-miR-551b-5p, hsa-miR-552, hsa-miR-553, hsa-miR-554, hsa-miR-557, hsa-miR-563, hsa-miR-564, hsa-miR-567, hsa-miR-569, hsa-miR-570-3p, hsa-miR-571, hsa-miR-572, hsa-miR-574-3p, hsa-miR-574-5p, hsa-miR-575, hsa-miR-576-3p, hsa-miR-576-5p, hsa-miR-577, hsa-miR-578, hsa-miR-580, hsa-miR-582-3p, hsa-miR-582-5p, hsa-miR-583, hsa-miR-584-5p, hsa-miR-585, hsa-miR-586, hsa-miR-589-3p, hsa-miR-589-5p, hsa-miR-590-3p, hsa-miR-590-5p, hsa-miR-593-3p, hsa-miR-593-5p, hsa-miR-595, hsa-miR-598, hsa-miR-601, hsa-miR-602, hsa-miR-603, hsa-miR-606, hsa-miR-608, hsa-miR-609, hsa-miR-611, hsa-miR-612, hsa-miR-613, hsa-miR-615-3p, hsa-miR-615-5p, hsa-miR-616-5p, hsa-miR-618, hsa-miR-619, hsa-miR-620, hsa-miR-623, hsa-miR-625-5p, hsa-miR-626, hsa-miR-627, hsa-miR-628-3p, hsa-miR-628-5p, hsa-miR-629-3p, hsa-miR-629-5p, hsa-miR-630, hsa-miR-631, hsa-miR-634, hsa-miR-635, hsa-miR-636, hsa-miR-638, hsa-miR-639, hsa-miR-641, hsa-miR-642a-3p, hsa-miR-642a-5p, hsa-miR-643, hsa-miR-645, hsa-miR-646, hsa-miR-647, hsa-miR-649, hsa-miR-650, hsa-miR-651, hsa-miR-652-3p, hsa-miR-653, hsa-miR-654-3p, hsa-miR-655, hsa-miR-656, hsa-miR-657, hsa-miR-658, hsa-miR-659-3p, hsa-miR-660-5p, hsa-miR-663a, hsa-miR-663b, hsa-miR-664a-3p, hsa-miR-664a-5p, hsa-miR-668, hsa-miR-671-5p, hsa-miR-675-3p, hsa-miR-675-5p, hsa-miR-7-1-3p, hsa-miR-7-2-3p, hsa-miR-7-5p, hsa-miR-708-3p, hsa-miR-708-5p, hsa-miR-718, hsa-miR-744-3p, hsa-miR-744-5p, hsa-miR-761, hsa-miR-765, hsa-miR-766, hsa-miR-767-3p, hsa-miR-769-3p, hsa-miR-769-5p, hsa-miR-770-5p, hsa-miR-874, hsa-miR-875, hsa-miR-876, hsa-miR-877-3p, hsa-miR-877-5p, hsa-miR-885-3p, hsa-miR-887, hsa-miR-888-3p, hsa-miR-889, hsa-miR-890, hsa-miR-891a, hsa-miR-891 b, hsa-miR-9-3p, hsa-miR-9-5p, hsa-miR-92a-3p, hsa-miR-92b-3p, hsa-miR-92b-5p, hsa-miR-921, hsa-miR-922, hsa-miR-93-3p, hsa-miR-93-5p, hsa-miR-935, hsa-miR-940, hsa-miR-941, hsa-miR-942, hsa-miR-95, hsa-miR-96-3p, hsa-miR-96-5p, hsa-miR-98-5p, hsa-miR-99a-3p, hsa-miR-99a-5p, hsa-miR-99b-3p, and hsa-miR-99b-5p, or is an antagomir or agomir thereof.

10-11. (canceled)

12. The composition of claim 1, wherein the targeting agent is an aptamer, an exosome, or a combination of an aptamer and an exosome.

13. (canceled)

14. The composition of claim 1113111, wherein the targeting agent is capable of binding to a cell surface marker selected from the group consisting of: CD9 (tetraspan), CD10 (MME), CD13 (ANPEP), CD29 (β-1 integrin), CD36 (FAT), CD44 (hyaluronate), CD49d (α-4 integrin), CD54 (ICAM-1), CD55 (DAF), CD59, CD73 (S-13), CD90 (Thy1), CD91 (LPR1), CD105 (SH12, Endoglin), CD137, CD146 (Muc 18), CD166 (ALCAM), HLA-ABC, adiponectin, caveolin-1, caveolin-2, CD36 (FAT), CLH-22 (clathrin heavy chain chromosome 22), DPT (dermatopotin), FABP4 (adipocyte protein 2, ap2), SLC27A1 (FATP1), SLC27A2 (FATP2), GLUT4 (glucose transporter 4), perilipin 2, resistin, neprilysin (CD10), FAT (CD36), Thy-1 (CD90), low density lipoprotein receptor-related protein 1 (LRP1, CD91), caveolin-1, caveolin-2, fatty acid binding protein 4 (FABP4), cell surface glycoprotein MUC18 (CD146), activated leukocyte cell adhesion molecule (CD166) and Natriuretic peptide receptor A (NPR1).

15. The composition of claim 1, wherein the targeting agent does not bind to a hematopoietic lineage marker selected from the group consisting of: CD11b (α-M integrin), CD14, CD18, CD19, CD31, CD34, CD45 (LCA), CD79 alpha, c-kit (ABCG2), STRO-1, HLA II, Lin1, Ter119, and HLA-DR.

16. The composition of claim 1, wherein the miRNA agent is covalently or non-covalently coupled to the targeting agent.

17-18. (canceled)

19. The composition of claim 16, wherein the miRNA agent is coupled to the targeting agent by a linker selected from the group consisting of: a polyalkylene glycol, polyethylene glycol, a dendrimer, a comb polymer, a biotinstreptavidin bridge, and a ribonucleic acid.

20. (canceled)

21. The composition of claim 1, further comprising a nanoparticle, wherein the nanoparticle has a diameter of no more than 100 nm.

22. (canceled)

23. The composition of claim 21, wherein the miRNA agent is encapsulated by the nanoparticle or is bound to the outside of the nanoparticle.

24. (canceled)

25. The composition of claim 21, wherein the nanoparticle is an exosome.

26. The composition of claim 1, further comprising at least a second miRNA agent.

27. The composition of claim 1, further comprising a pharmaceutically acceptable carrier.

28-30. (canceled)

31. A method for delivering a therapeutic microRNA (miRNA) to a specific cell, tissue, or organ in a subject comprising administering the composition of claim 1 to the subject.

32-36. (canceled)

37. A method for treating obesity or metabolic disorders in a subject, comprising administering the composition of claim 1 to the subject.

38. The method of claim 37, wherein the aptamir composition is delivered to an adipocyte cell or to an adipose tissue derived mesenchymal stem cell in the subject.

39. (canceled)

40. The method of claim 37, wherein the miRNA agent modulates activity of at least one mitochondrial uncoupler selected from UCP1, UCP2, and UCP3.

41. (canceled)

42. The method of claim 40, wherein the miRNA modulator directly binds to the 5′UTR or coding sequence of the mRNA of at least one mitochondrial uncoupler or to the promoter region of at least one mitochondrial uncoupler.

43. (canceled)
